(19) United States
(12) Patent Application Publication
(10) Pub. No.: US 2024/0244860 A1
(43) Pub. Date: Jul. 18, 2024

(54) LIGHT EMITTING ELEMENT AND AMINE COMPOUND FOR THE SAME

(71) Applicant: Samsung Display Co., Ltd., Yongin-si (KR)

(72) Inventors: Takao SAKUMA, Yokohama (JP); XiuLan JIN, Yokohama (JP); Ichinori TAKADA, Yokohama (JP)

(21) Appl. No.: 18/532,924

(22) Filed: Dec. 7, 2023

(30) Foreign Application Priority Data
Dec. 23, 2022 (KR) 10-2022-0182966

(51) Int. Cl. H10K 50/11; C09K 11/06; H10K 50/15; H10K 50/16; H10K 85/60

(52) U.S. Cl. CPC H10K 50/11; C09K 11/06; H10K 50/15; H10K 50/16; H10K 85/633; C09K 2211/1018

(57) ABSTRACT

A light emitting element and an amine compound for a light emitting element is provided. The light emitting element includes a first electrode, a second electrode on the first electrode, and at least one functional layer (e.g., hole transport layer) between the first electrode and the second electrode. The functional layer includes a tertiary amine compound represented by a specific chemical structure (i.e., Formula 1). The tertiary amine compound is configured so that the hole transport properties of the functional layer (e.g., hole transport layer) and the emission efficiency and lifetime of the light emitting element may be enhanced or improved.

### CROSS-REFERENCE TO RELATED APPLICATION

This application claims priority to and the benefit of Korean Patent Application No. 10-2022-0182966, filed on Dec. 23, 2022, in the Korean Intellectual Property Office, the entire content of which is hereby incorporated by reference.

### BACKGROUND

### 1. Field

One or more aspects of embodiments of the present disclosure relate to a light emitting element and an amine compound for a light emitting element, and particularly, to a light emitting element including an amine compound in a functional layer.

### 2. Description of the Related Art

Recently, the development of an organic electroluminescence display device to be utilized as an image display device has been actively conducted. The organic electroluminescence display device includes a “self-luminescent light emitting element” that enables display of images by recombining holes and electrons injected from a first electrode and a second electrode in an emission layer. Subsequently, a light emitting material in the emission layer emits light to achieve display.

Implementation of the organic electroluminescence device in a display device requires (or there is a desire) that the light emitting element (e.g., self-luminescent light emitting element) possess reduced driving voltage and an improved emission efficiency and/or long lifetime. Therefore, the need exists for the development of materials for a light emitting element capable of stably (or suitably) implementing these properties. For example, in an effort to implement a light emitting element having high emission efficiency and long lifetime, the development of materials for a hole transport region having excellent or suitable hole transport properties and stability is being conducted.

### SUMMARY

One or more aspects of embodiments of the present disclosure is directed toward a light emitting element showing long-life (e.g., long lifetime) characteristics and an amine compound which is included in the light emitting element. Additional aspects will be set forth in part in the description which follows and, in part, will be apparent from the description, or may be learned by practice of the presented embodiments of the disclosure.

A light emitting element of one or more embodiments of the present disclosure may include a first electrode, a second electrode provided on the first electrode, and an amine compound of one or more embodiments in at least one functional layer provided between the first electrode and the second electrode.

The amine compound of one or more embodiments may be represented by Formula 1.

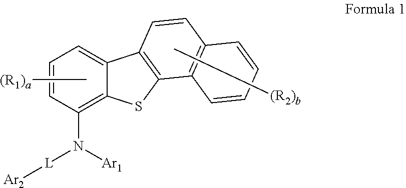

In Formula 1, R₁ and R₂ may each independently be a hydrogen atom, a deuterium atom, a substituted or unsubstituted alkyl group of 1 to 10 carbon atoms, a substituted or unsubstituted aryl group of 6 to 30 ring-forming carbon atoms, or a substituted or unsubstituted heteroaryl group of 5 to 30 ring-forming carbon atoms. L may be a direct linkage, a substituted or unsubstituted arylene group of 6 to 30 ring-forming carbon atoms, or a substituted or unsubstituted heteroarylene group of 5 to 30 ring-forming carbon atoms. In Formula 1, “a” may be an integer of 0 to 3, and “b” may be an integer of 0 to 6. Ar₁ may be represented by Formula 2, and Ar₂ may be a substituted or unsubstituted aryl group of 6 to 30 ring-forming carbon atoms, or a substituted or unsubstituted heteroaryl group of 5 to 30 ring-forming carbon atoms. Here, a case where Ar₂ is a substituted or unsubstituted carbazole group, is excluded.

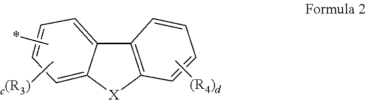

In Formula 2, X may be O, S, or CR₅R₆. R₃ and R₄ may each independently be a hydrogen atom, a deuterium atom, a substituted or unsubstituted alkyl group of 1 to 10 carbon atoms, a substituted or unsubstituted aryl group of 6 to 30 ring-forming carbon atoms, or a substituted or unsubstituted heteroaryl group of 5 to 30 ring-forming carbon atoms. R₅ and R₆ may each independently be a substituted or unsubstituted aryl group of 6 to 30 ring-forming carbon atoms, or a substituted or unsubstituted heteroaryl group of 5 to 30 ring-forming carbon atoms. “c” may be an integer of 0 to 3, and “d” may be an integer of 0 to 4. When X is O or S and is combined at an ortho position or a para position with respect to a nitrogen atom of an amine, a case where -L-Ar₂ of Formula 1 is an unsubstituted phenyl group, an unsubstituted biphenyl group, an unsubstituted terphenyl group, or an unsubstituted 2-phenanthrenyl group, may be excluded, and a case where each of R₃ and R₄ is a substituted or unsubstituted 9-fluorenyl group, or a substituted or unsubstituted phenazine group, may be excluded. “-*” refers to a position to be connected.

In one or more embodiments, the amine compound represented by Formula 1 may be represented by any one selected from among Formula 3-1 to Formula 3-3.

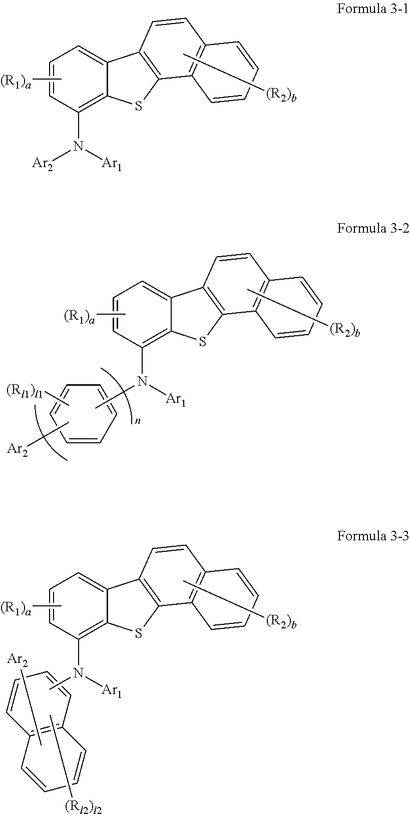

In Formula 3-2 and Formula 3-3, R_{I1} and R_{I2} may each independently be a hydrogen atom, a deuterium atom, a substituted or unsubstituted alkyl group of 1 to 10 carbon atoms, a substituted or unsubstituted aryl group of 6 to 30 ring-forming carbon atoms, or a substituted or unsubstituted heteroaryl group of 5 to 30 ring-forming carbon atoms, 11 may be an integer of 0 to 4, 12 may be an integer of 0 to 6, and “n” may be an integer of 1 to 3. In Formula 3-1 to Formula 3-3, R₁, R₂, Ar₁, Ar₂, “a” and “b” may each independently be substantially the same as defined in Formula 1.

In one or more embodiments, the amine compound represented by Formula 1 may be represented by any one selected from among Formula 4-1 to Formula 4-3.

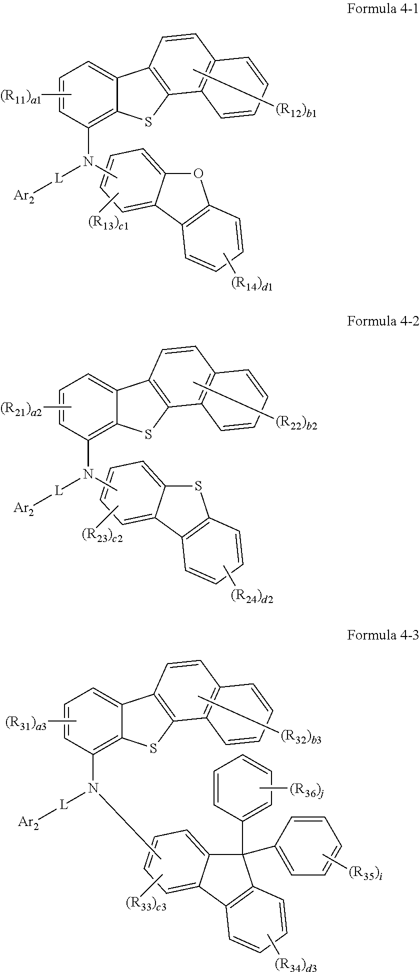

In Formula 4-1 to Formula 4-3, R₁₁ to R₁₄, R₂₁ to R₂₄, and R₃₁ to R₃₆ may each independently be a hydrogen atom or a deuterium atom, a1 to a3, and c1 to c3 may each independently be an integer of 0 to 3, b1 to b3 may each independently be an integer of 0 to 6, d1 to d3 may each independently be an integer of 0 to 4, “i” and “j” may each independently be an integer of 0 to 5, and Ar₂ and L may each independently be substantially the same as defined in Formula 1.

In one or more embodiments, the amine compound represented by Formula 1 may be represented by any one selected from among Formula 5-1 and/or Formula 5-2 (e.g., Formula 1 may be Formula 5-1 or Formula 5-2).

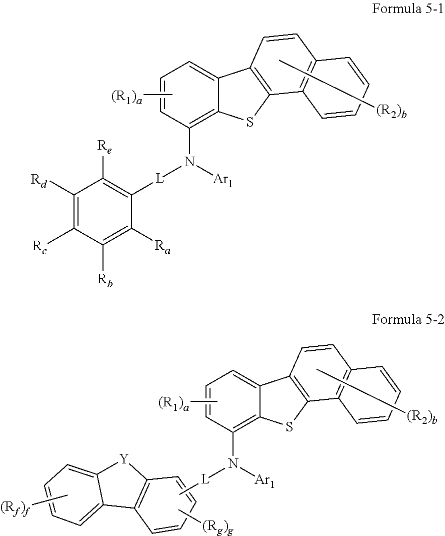

In Formula 5-1, Rₐ to Rₑ may each independently be a hydrogen atom, a deuterium atom, or a substituted or unsubstituted aryl group of 6 to 20 ring-forming carbon atoms. In one or more embodiments, elements (e.g., atoms or portions) of at least one (e.g., one pair) selected from among Rₐ and R_{b}, R_{b} and R_{c}, R_{c} and R_{d}, and/or Rₐ and Rₑ may form (e.g., may be combined with each other to form) a substituted or unsubstituted aromatic hydrocarbon ring. In Formula 5-2, Y may be O, S or CR_{y1}R_{y2}, R_{f} and R_{g} may each independently be a hydrogen atom or a deuterium atom, R_{y1} and R_{y2} may each independently be a substituted or unsubstituted alkyl group of 1 to 10 carbon atoms, or a substituted or unsubstituted aryl group of 6 to 15 ring-forming carbon atoms, and/or R_{y1} and R_{y2} may form (e.g., are combined with each other to form) a ring, “f” is an integer of 0 to 4, and “g” is an integer of 0 to 3. In Formula 5-1 and Formula 5-2, R₁, R₂, L, Ar₁, “a” and “b” may each independently be substantially the same as defined in Formula 1.

In one or more embodiments, Ar₂ may be a substituted or unsubstituted phenyl group, a substituted or unsubstituted biphenyl group, a substituted or unsubstituted terphenyl group, a substituted or unsubstituted quaterphenyl group, a substituted or unsubstituted naphthyl group, a substituted or unsubstituted phenanthrenyl group, a substituted or unsubstituted dibenzofuran group, a substituted or unsubstituted dibenzothiophene group, or a substituted or unsubstituted fluorenyl group.

In one or more embodiments, the amine compound represented by Formula 1 may be a monoamine compound.

In one or more embodiments, Ar₁ may be represented by any one selected from among substituents in Substituent Group 1, as described elsewhere herein.

In one or more embodiments, -L-Ar₂ of Formula 1 may be represented by any one selected from among substituents in Substituent Group 2, as described elsewhere herein.

In one or more embodiments, R₁ and R₂ may be hydrogen atoms.

In one or more embodiments, the at least one functional layer may include a hole transport region located or disposed on the first electrode, an emission layer located or disposed on the hole transport region, and an electron transport region located or disposed on the emission layer, and the hole transport region may include the amine compound represented by Formula 1.

In one or more embodiments, the hole transport region may include a hole injection layer located or disposed on the first electrode, and a hole transport layer located or disposed on the hole injection layer, and the hole transport layer may include the amine compound represented by Formula 1.

In one or more embodiments, the emission layer may include a compound represented by Formula E-1.

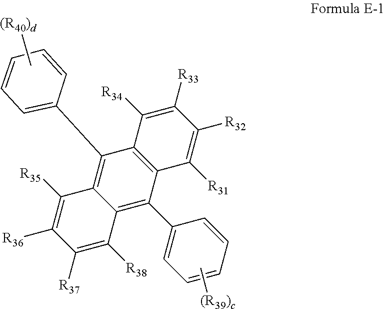

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring, and “c” and “d” may each independently be an integer of 0 to 5.

### BRIEF DESCRIPTION OF THE DRAWINGS

The accompanying drawings are included to provide a further understanding of the present disclosure and are incorporated in and constitute a part of this specification. The drawings illustrate example embodiments of the present disclosure and, together with the description, serve to explain principles of the present disclosure. In the drawings:

FIG. 1 is a plan view showing a display device according to one or more embodiments of the present disclosure;

FIG. 2 is a cross-sectional view of a display device according to one or more embodiments of the present disclosure;

FIG. 3 is a cross-sectional view schematically showing a light emitting element according to one or more embodiments of the present disclosure;

FIG. 4 is a cross-sectional view schematically showing a light emitting element according to one or more embodiments of the present disclosure;

FIG. 5 is a cross-sectional view schematically showing a light emitting element according to one or more embodiments of the present disclosure;

FIG. 6 is a cross-sectional view schematically showing a light emitting element according to one or more embodiments of the present disclosure;

FIG. 7 is a cross-sectional view of a display device according to one or more embodiments of the present disclosure;

FIG. 8 is a cross-sectional view of a display device according to one or more embodiments of the present disclosure;

FIG. 9 is a cross-sectional view showing a display device according to one or more embodiments of the present disclosure;

FIG. 10 is a cross-sectional view showing a display device according to one or more embodiments of the present disclosure; and

FIG. 11 is a diagram showing a vehicle including a display device according to one or more embodiments of the present disclosure.

### DETAILED DESCRIPTION

The present disclosure may have one or more suitable modifications and may be embodied in different forms, and example embodiments will be explained in more detail with reference to the accompany drawings. The present disclosure may, however, be embodied in different forms and should not be construed as limited to the embodiments set forth herein. Rather, all modifications, equivalents, and substituents which are included in the spirit and technical scope of the present disclosure should be included in the present disclosure. Unless otherwise defined, all chemical names, technical and scientific terms, and terms defined in common dictionaries should be interpreted as having meanings consistent with the context of the related art, and should not be interpreted in an ideal or overly formal sense.

Like reference numerals refer to like elements throughout, and duplicative descriptions thereof may not be provided. In the drawings, the dimensions of structures are exaggerated for clarity of illustration. It will be understood that, although the terms first, second, etc. may be utilized herein to describe one or more suitable elements, these elements should not be limited by these terms. These terms are only utilized to distinguish one element from another element. Thus, a first element could be termed a second element without departing from the teachings of the present disclosure. Similarly, a second element could be termed a first element. As utilized herein, the singular forms are intended to include the plural forms as well, unless the context clearly indicates otherwise.

In the application, it will be further understood that the terms “comprise,” “comprises,” “comprising,” “has,” “have,” “having,” “include,” “includes,” and/or “including,” when utilized in this specification, specify the presence of stated features, numerals, steps, operations, elements, parts, or the combination thereof, but do not preclude the presence or addition of one or more other features, numerals, steps, operations, elements, parts, or the combination thereof.

As used herein, the terms “use,” “using,” and “used” may be considered synonymous with the terms “utilize,” “utilizing,” and “utilized,” respectively.

As used herein, expressions such as “at least one of,” “one of,” and “selected from,” when preceding a list of elements, modify the entire list of elements and do not modify the individual elements of the list. For example, the expressions “at least one of a to c,” “at least one of a, b or c,” and “at least one of a, b and/or c” may indicate only a, only b, only c, both (e.g., simultaneously) a and b, both (e.g., simultaneously) a and c, both (e.g., simultaneously) b and c, all of a, b, and c, or variations thereof.

As used herein, the term “and/or” includes any and all combinations of one or more of the associated listed items.

The term “may” will be understood to refer to “one or more embodiments of the present disclosure,” some of which include the described element and some of which exclude that element and/or include an alternate element. Similarly, alternative language such as “or” refers to “one or more embodiments of the present disclosure,” each including a corresponding listed item.

It will be understood that when an element is referred to as being “on,” “connected to,” or “coupled to” another element, it may be directly on, connected, or coupled to the other element or one or more intervening elements may also be present. When an element is referred to as being “directly on,” “directly connected to,” or “directly coupled to” another element, there are no intervening elements present.

Spatially relative terms, such as “beneath,” “below,” “lower,” “above,” “upper,” “bottom,” “top,” and the like, may be used herein for ease of description to describe one element or feature's relationship to another element(s) or feature(s) as illustrated in the drawings. It will be understood that the spatially relative terms are intended to encompass different orientations of the device in use or operation in addition to the orientation depicted in the drawings. For example, if the device in the drawings is turned over, elements described as “below” or “beneath” other elements or features would then be oriented “above” or “over” the other elements or features. Thus, the term “below” may encompass both an orientation of above and below. The device may be otherwise oriented (rotated 90 degrees or at other orientations), and the spatially relative descriptors used herein should be interpreted accordingly.

Unless otherwise defined, all terms (including technical and scientific terms) used herein have the same meaning as commonly understood by those of ordinary skill in the art to which the present disclosure pertains. It is also to be understood that terms defined in commonly used dictionaries should be interpreted as having meanings consistent with meanings in the context of the related art, unless expressly defined herein, and should not be interpreted in an ideal or overly formal sense.

In this context, “consisting essentially of” means that any additional components will not materially affect the chemical, physical, optical, or electrical properties of the semiconductor film.

Further, in this specification, the phrase “on a plane,” or “plan view,” means viewing a target portion from the top, and the phrase “on a cross-section” means viewing a cross-section formed by vertically cutting a target portion from the side.

### Definitions

In the description, the term “substituted or unsubstituted” corresponds to substituted or unsubstituted with at least one substituent selected from the group consisting of a deuterium atom, a halogen atom, a cyano group, a nitro group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, an alkoxy group, a hydrocarbon ring group, an aryl group, and a heterocyclic group. In some embodiments, each of the exemplified substituents may be substituted or unsubstituted. For example, a biphenyl group may be interpreted as an aryl group or a phenyl group substituted with a phenyl group.

In the description, the term “forming a ring via the combination with an adjacent group” may refer to forming a substituted or unsubstituted hydrocarbon ring, or a substituted or unsubstituted heterocycle via the combination with an adjacent group. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocycles or polycycles. In some embodiments, the ring formed via the combination with an adjacent group may be combined with another ring to form a spiro structure.

In the description, the term “adjacent group” may refer to a substituent substituted for an atom which is directly combined with an atom substituted with a corresponding substituent, another substituent substituted for an atom which is substituted with a corresponding substituent, or a substituent sterically positioned at the nearest position to a corresponding substituent. For example, in 1,2-dimethylbenzene, two methyl groups may be interpreted as “adjacent groups” to each other, and in 1,1-diethylcyclopentane, two ethyl groups may be interpreted as “adjacent groups” to each other. In some embodiments, in 4,5-dimethylphenanthrene, two methyl groups may be interpreted as “adjacent groups” to each other.

In the description, a halogen atom may be a fluorine atom, a chlorine atom, a bromine atom or an iodine atom.

In the description, an alkyl group may be a linear, branched, or cyclic type or kind. The carbon number of the alkyl group may be 1 to 50, 1 to 30, 1 to 20, 1 to 10, or 1 to 6. Examples of the alkyl group may include methyl group, ethyl group, n-propyl group, isopropyl group, n-butyl group, s-butyl group, t-butyl group, i-butyl group, 2-ethylbutyl group, 3,3-dimethylbutyl group, n-pentyl group, i-pentyl group, neopentyl group, t-pentyl group, cyclopentyl group, 1-methylpentyl group, 3-methylpentyl group, 2-ethylpentyl group, 4-methyl-2-pentyl group, n-hexyl group, 1-methylhexyl group, 2-ethylhexyl group, 2-butylhexyl group, cyclohexyl group, 4-methylcyclohexyl group, 4-t-butylcyclohexyl group, n-heptyl group, 1-methylheptyl group, 2,2-dimethylheptyl group, 2-ethylheptyl group, 2-butylheptyl group, n-octyl group, t-octyl group, 2-ethyloctyl group, 2-butyloctyl group, 2-hexyloctyl group, 3,7-dimethyloctyl group, cyclooctyl group, n-nonyl group, n-decyl group, adamantyl group, 2-ethyldecyl group, 2-butyldecyl group, 2-hexyldecyl group, 2-octyldecyl group, n-undecyl group, n-dodecyl group, 2-ethyldodecyl group, 2-butyldodecyl group, 2-hexyldodecyl group, 2-octyldodecyl group, n-tridecyl group, n-tetradecyl group, n-pentadecyl group, n-hexadecyl group, 2-ethylhexadecyl group, 2-butylhexadecyl group, 2-hexylhexadecyl group, 2-octylhexadecyl group, n-heptadecyl group, n-octadecyl group, n-nonadecyl group, n-eicosyl group, 2-ethyleicosyl group, 2-butyleicosyl group, 2-hexyleicosyl group, 2-octyleicosyl group, n-heneicosyl group, n-docosyl group, n-tricosyl group, n-tetracosyl group, n-pentacosyl group, n-hexacosyl group, n-heptacosyl group, n-octacosyl group, n-nonacosyl group, n-triacontyl group, etc., without limitation.

In the description, a cycloalkyl group may refer to a ring-type or kind alkyl group. The carbon number of the cycloalkyl group may be 3 to 50, 3 to 30, 3 to 20, or 3 to 10. Examples of the cycloalkyl group include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group etc., without limitation.

In the description, an alkenyl group refers to a hydrocarbon group including one or more carbon double bonds in the middle or at the terminal of an alkyl group having a carbon number of 2 or more. The alkenyl group may be a linear chain or a branched chain. The carbon number is not specifically limited, but may be 2 to 30, 2 to 20, or 2 to 10. Examples of the alkenyl group include a vinyl group, an allyl group, a 1-butenyl group, a 1-pentenyl group, a 1,3-butadienyl group, a styrenyl group, a styrylvinyl group, etc., without limitation.

In the description, an aryl group refers to an optional functional group or substituent derived from an aromatic hydrocarbon ring. The aryl group may be a monocyclic aryl group or a polycyclic aryl group. The carbon number for forming rings in the aryl group may be 6 to 50, 6 to 40, 6 to 30, 6 to 20, or 6 to 15. Examples of the aryl group may include phenyl, naphthyl, fluorenyl, anthracenyl, phenanthryl, biphenyl, terphenyl, quaterphenyl, quinquephenyl, sexiphenyl, triphenylenyl, pyrenyl, benzofluoranthenyl, chrysene, etc., without limitation.

In the description, a fluorenyl group may be substituted, and two substituents may be combined with each other to form a spiro structure. Examples of a substituted fluorenyl group are as follows, but one or more embodiments of the present disclosure is not limited thereto.

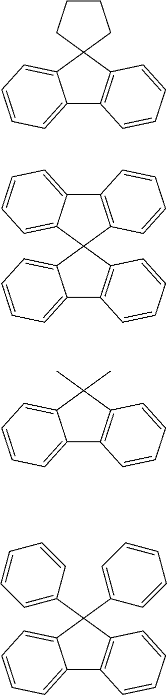

In the description, a heterocyclic group refers to an optional functional group or substituent derived from a ring including one or more among B, O, N, P, Si, Se and S as heteroatoms. The heterocyclic group includes an aliphatic heterocyclic group and an aromatic heterocyclic group. The aromatic heterocyclic group may be a heteroaryl group. The aliphatic heterocyclic group and the aromatic heterocyclic group may be a monocycle or a polycycle.

In the description, a heteroaryl group may include one or more among B, O, N, P, Si, Se and S as heteroatoms. When the heteroaryl group includes two or more heteroatoms, two or more heteroatoms may be the same or different. The heteroaryl group may be a monocyclic heterocyclic group or polycyclic heterocyclic group. The carbon number for forming rings of the heteroaryl group may be 2 to 50, 2 to 40, 2 to 30, 2 to 20, or 2 to 10. Examples of the heteroaryl group may include thiophene, furan, pyrrole, imidazole, pyridine, bipyridine, pyrimidine, triazine, triazole, acridyl, pyridazine, pyrazinyl, quinoline, quinazoline, quinoxaline, phenoxazine, phthalazine, pyrido pyrimidine, pyrido pyrazine, pyrazino pyrazine, isoquinoline, indole, carbazole, N-arylcarbazole, N-heteroarylcarbazole, N-alkylcarbazole, benzoxazole, benzimidazole, benzothiazole, benzocarbazole, benzothiophene, dibenzothiophene, thienothiophene, benzofuran, phenanthroline, thiazole, isooxazole, oxazole, oxadiazole, thiadiazole, phenothiazine, dibenzosilole, dibenzofuran, etc., without limitation.

In the description, the same explanation on the above-described aryl group may be applied to an arylene group except that the arylene group is a divalent group. The same explanation on the above-described heteroaryl group may be applied to a heteroarylene group except that the heteroarylene group is a divalent group.

In the description, a silyl group includes an alkyl silyl group and an aryl silyl group. Examples of the silyl group include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, etc., without limitation.

In the description, a thio group may include an alkyl thio group and an aryl thio group. The thio group may refer to the above-defined alkyl group or aryl group combined with a sulfur atom. Examples of the thio group include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, etc., without limitation.

In the description, an oxy group may refer to the above-defined alkyl group or aryl group which is combined with an oxygen atom. The oxy group may include an alkoxy group and an aryl oxy group. The alkoxy group may be a linear, branched or cyclic chain. The carbon number of the alkoxy group is not specifically limited but may be, for example, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, etc. However, one or more embodiments of the present disclosure is not limited thereto.

In the description, a boron group may refer to the above-defined alkyl group or aryl group combined with a boron atom. The boron group includes an alkyl boron group and an aryl boron group. Examples of the boron group include a dimethylboron group, a diethylboron group, a t-butylboron group, a diphenylboron group, a diphenylboron group, a phenylboron group, and/or the like, without limitation.

In the description, the carbon number of an amine group is not specifically limited, but may be 1 to 30. The amine group may include an alkyl amine group and an aryl amine group. Examples of the amine group include a methylamine group, a dimethylamine group, a phenylamine group, a diphenylamine group, a naphthylamine group, a 9-methyl-anthracenylamine group, etc., without limitation.

In the description, a direct linkage may refer to a single bond.

In some embodiments, in the description, “ [symbol] ”, “-*”, “ [symbol] ” and “ [symbol] ” refer to positions to be connected.

Hereinafter, the light emitting element according to one or more embodiments will be explained referring to the drawings.

### Display Device

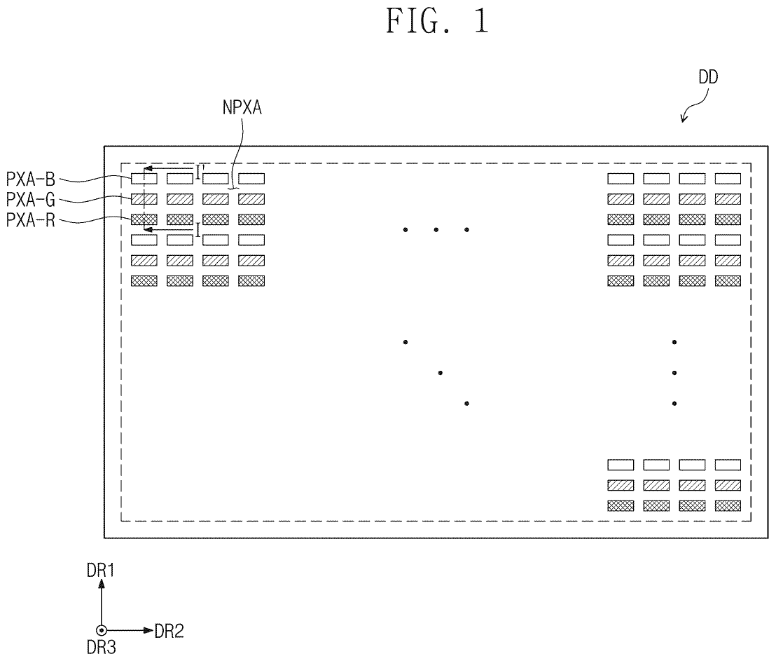

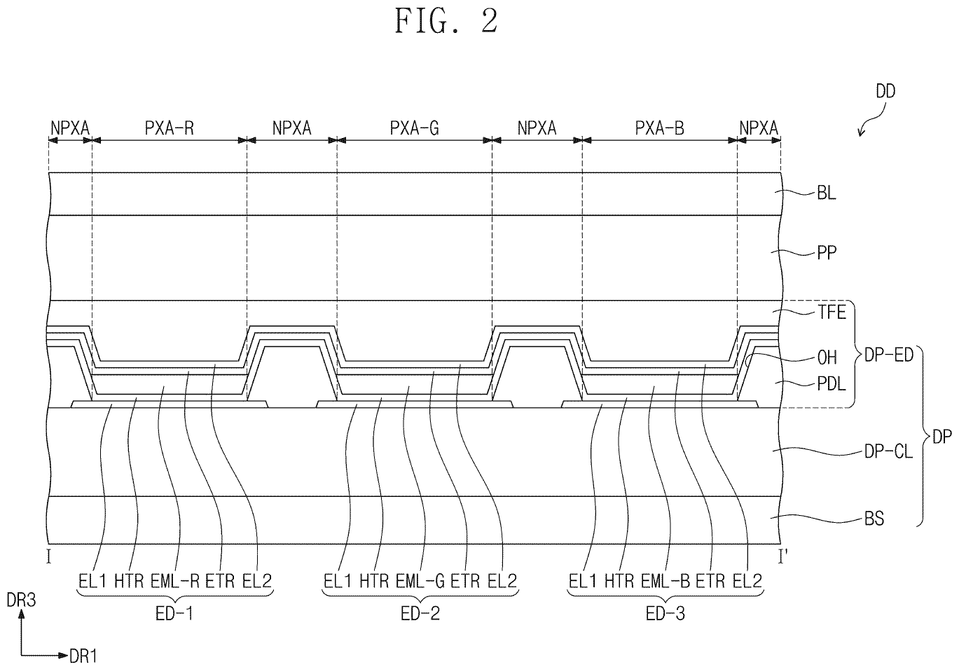

FIG. 1 is a plan view showing one or more embodiments of a display device DD. FIG. 2 is a cross-sectional view of a display device DD of one or more embodiments showing a part corresponding to line I-I′ of FIG. 1.

The display device DD may include a display panel DP and an optical layer PP provided on the display panel DP. The display panel DP may include light emitting elements ED-1, ED-2 and ED-3. The display device DD may include multiple light emitting elements ED-1, ED-2 and ED-3. The optical layer PP may be provided on the display panel DP and control reflected light by external light at the display panel DP. The optical layer PP may include, for example, a polarization layer or a color filter layer. In some embodiments, different from the drawings, the optical layer PP may not be provided in the display device DD of one or more embodiments.

On the optical layer PP, a base substrate BL may be provided. The base substrate BL may be a member providing a base surface where the optical layer PP is provided. The base substrate BL may be a glass substrate, a metal substrate, a plastic substrate, and/or the like. However, one or more embodiments of the present disclosure is not limited thereto, and the base substrate BL may be an inorganic layer, an organic layer and/or a composite material layer. In some embodiments, different from the drawings, the base substrate BL may not be provided in one or more embodiments.

The display device DD according to one or more embodiments may further include a filling layer. The filling layer may be provided between a display element layer DP-ED and a base substrate BL. The filling layer may be an organic layer. The filling layer may include at least one among an acrylic-based resin, a silicon-based resin and an epoxy-based resin.

The display panel DP may include a base layer BS, a circuit layer DP-CL provided on the base layer BS and a display element layer DP-ED. The display element layer DP-ED may include a pixel definition layer PDL, light emitting elements ED-1, ED-2 and ED-3 provided in the pixel definition layer PDL, and an encapsulating layer TFE provided on the light emitting elements ED-1, ED-2 and ED-3.

The base layer BS may be a member providing a base surface where the display element layer DP-ED is provided. The base layer BS may be a glass substrate, a metal substrate, a plastic substrate, and/or the like. However, one or more embodiments of the present disclosure is not limited thereto, and the base layer BS may be an inorganic layer, an organic layer and/or a composite material layer.

In one or more embodiments, the circuit layer DP-CL is provided on the base layer BS, and the circuit layer DP-CL may include multiple transistors. Each of the transistors may include a control electrode, an input electrode, and an output electrode. For example, the circuit layer DP-CL may include switching transistors and driving transistors for driving the light emitting elements ED-1, ED-2 and ED-3 of the display element layer DP-ED.

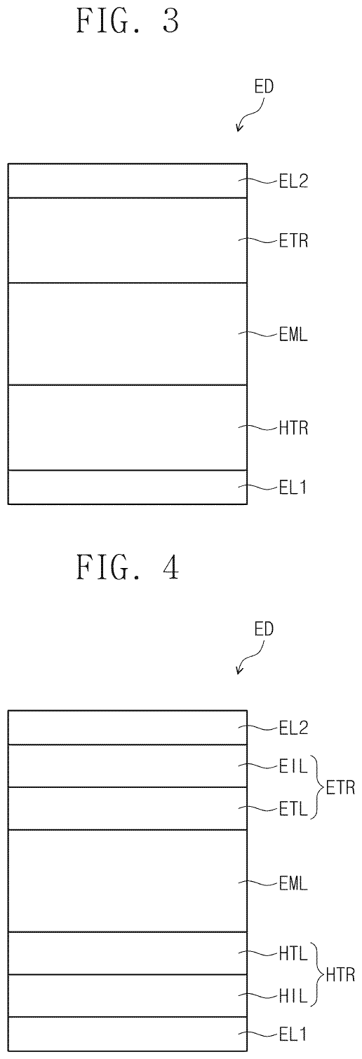

The light emitting elements ED-1, ED-2 and ED-3 may have the structures of the light emitting elements ED of embodiments according to FIG. 3 to FIG. 6, as described in more detail elsewhere herein. The light emitting elements ED-1, ED-2 and ED-3 may include a first electrode EL1, a hole transport region HTR, emission layers EML-R, EML-G and EML-B, an electron transport region ETR and a second electrode EL2.

In FIG. 2, shown is one or more embodiments where the emission layers EML-R, EML-G and EML-B of light emitting elements ED-1, ED-2 and ED-3 are provided in opening portions OH defined in a pixel definition layer PDL, and a hole transport region HTR, an electron transport region ETR and a second electrode EL2 are provided as common layers in all light emitting elements ED-1, ED-2 and ED-3. However, one or more embodiments of the present disclosure is not limited thereto. Different from FIG. 2, in one or more embodiments, the hole transport region HTR and the electron transport region ETR may be patterned and provided in the opening portions OH defined in the pixel definition layer PDL. For example, in one or more embodiments, the hole transport region HTR, the emission layers EML-R, EML-G and EML-B, and the electron transport region ETR of the light emitting elements ED-1, ED-2 and ED-3 may be patterned or formed by an inkjet printing method and provided.

An encapsulating layer TFE may cover the light emitting elements ED-1, ED-2 and ED-3. The encapsulating layer TFE may encapsulate the light emitting elements ED-1, ED-2 and ED-3 in the display element layer DP-ED. The encapsulating layer TFE may be a thin film encapsulating layer. The encapsulating layer TFE may be one layer or a stacked layer of multiple layers. The encapsulating layer TFE includes at least one insulating layer. The encapsulating layer TFE according to one or more embodiments may include at least one inorganic layer (hereinafter, encapsulating inorganic layer). In some embodiments, the encapsulating layer TFE according to one or more embodiments may include at least one organic layer (hereinafter, encapsulating organic layer) and at least one encapsulating inorganic layer.

The encapsulating inorganic layer protects the display element layer DP-ED from moisture and/or oxygen, and the encapsulating organic layer protects the display element layer DP-ED from foreign materials such as dust particles. The encapsulating inorganic layer may include silicon nitride, silicon oxy nitride, silicon oxide, titanium oxide, aluminum oxide, and/or the like. The encapsulating organic layer may include an acrylic-based compound, an epoxy-based compound, and/or the like. The encapsulating organic layer may include a photopolymerizable organic material, and/or the like.

The encapsulating layer TFE may be provided on the second electrode EL2 and may be provided while filling the opening portion OH.

Referring to FIG. 1 and FIG. 2, the display device DD may include a non-luminous area NPXA and luminous areas PXA-R, PXA-G and PXA-B. The luminous areas PXA-R, PXA-G and PXA-B may be areas emitting light produced from the light emitting elements ED-1, ED-2 and ED-3, respectively. The luminous areas PXA-R, PXA-G and PXA-B may be separated from each other, e.g., when viewed on a plane.

The luminous areas PXA-R, PXA-G and PXA-B may be areas separated by the pixel definition layer PDL. The non-luminous areas NPXA may be areas between neighboring luminous areas PXA-R, PXA-G and PXA-B and may be areas corresponding to the pixel definition layer PDL. In some embodiments, in the disclosure, each of the luminous areas PXA-R, PXA-G and PXA-B may correspond to each pixel. The pixel definition layer PDL may divide the light emitting elements ED-1, ED-2 and ED-3. The emission layers EML-R, EML-G and EML-B of the light emitting elements ED-1, ED-2 and ED-3 may be provided and divided in the opening portions OH defined in the pixel definition layer PDL.

The luminous areas PXA-R, PXA-G and PXA-B may be divided into multiple groups according to the color of light produced from the light emitting elements ED-1, ED-2 and ED-3. In the display device DD of one or more embodiments, shown in FIG. 1 and FIG. 2, three luminous areas PXA-R, PXA-G and PXA-B emitting red light, green light and blue light are illustrated as one or more embodiments. For example, the display device DD of one or more embodiments may include a red luminous area PXA-R, a green luminous area PXA-G and a blue luminous area PXA-B, which are separated from each other.

In the display device DD according to one or more embodiments, multiple light emitting elements ED-1, ED-2 and ED-3 may be to emit light having different wavelength regions. For example, in one or more embodiments, the display device DD may include a first light emitting element ED-1 emitting red light, a second light emitting element ED-2 emitting green light, and a third light emitting element ED-3 emitting blue light. For example, the red luminous area PXA-R, the green luminous area PXA-G, and the blue luminous area PXA-B of the display device DD may correspond to the first light emitting element ED-1, the second light emitting element ED-2, and the third light emitting element ED-3, respectively.

However, one or more embodiments of the present disclosure is not limited thereto, and the first to third light emitting elements ED-1, ED-2 and ED-3 may be to emit light in substantially the same wavelength region, or at least one thereof may be to emit light in a different wavelength region. For example, all the first to third light emitting elements ED-1, ED-2 and ED-3 may be to emit blue light.

The luminous areas PXA-R, PXA-G and PXA-B in the display device DD according to one or more embodiments may be arranged in a stripe shape. Referring to FIG. 1, multiple red luminous areas PXA-R may be arranged with each other along a second directional axis DR2, multiple green luminous areas PXA-G may be arranged with each other along the second directional axis DR2 and/or multiple blue luminous areas PXA-B may be arranged with each other along the second directional axis DR2. In some embodiments, the (one) red luminous area PXA-R, the (one) green luminous area PXA-G and the (one) blue luminous area PXA-B may be arranged with each other by turns along a first directional axis DR1. A third directional axis DR3 may be perpendicular to a plane defined by the first directional axis DR1 and the second directional axis DR2.

In FIG. 1 and FIG. 2, the areas of the luminous areas PXA-R, PXA-G and PXA-B are shown similar, but one or more embodiments of the present disclosure is not limited thereto. The areas of the luminous areas PXA-R, PXA-G and PXA-B may be different from each other according to the wavelength region of light emitted. In some embodiments, the areas of the luminous areas PXA-R, PXA-G and PXA-B may refer to areas on a plane defined by the first directional axis DR1 and the second directional axis DR2 (or areas in a plan view).

In some embodiments, the arrangement type or kind of the luminous areas PXA-R, PXA-G and PXA-B is not limited to the configuration shown in FIG. 1, and the arrangement order of the red luminous areas PXA-R, the green luminous areas PXA-G and the blue luminous areas PXA-B may be provided in one or more suitable combinations according to the properties of display quality required for the display device DD. For example, the arrangement type or kind of the luminous areas PXA-R, PXA-G and PXA-B may be the PENTILE® arrangement form, or the Diamond Pixel™ arrangement form. (PENTILE® is a registered trademark owned by Samsung Display Co., Ltd.)

In some embodiments, the size(s) or area(s) of the luminous areas PXA-R, PXA-G and PXA-B may be different from each other. For example, in one or more embodiments, the area of the green luminous area PXA-G may be smaller than the area of the blue luminous area PXA-B, but one or more embodiments of the present disclosure is not limited thereto.

FIG. 3 to FIG. 6 are each a cross-sectional view schematically showing a light emitting element according to one or embodiments. The light emitting element ED according to one or more embodiments may include a first electrode EL1, a second electrode EL2 oppositely provided to the first electrode EL1, and at least one functional layer provided between the first electrode EL1 and the second electrode EL2. The light emitting element ED of one or more embodiments may include an amine compound of one or more embodiments, as described elsewhere herein, in the at least one functional layer.

The light emitting element ED may include a hole transport region HTR, an emission layer EML, an electron transport region ETR, and/or the like, stacked in order, as the at least one functional layer. Referring to FIG. 3, the light emitting element ED of one or more embodiments may include a first electrode EL1, a hole transport region HTR, an emission layer EML, an electron transport region ETR, and a second electrode EL2, stacked in order.

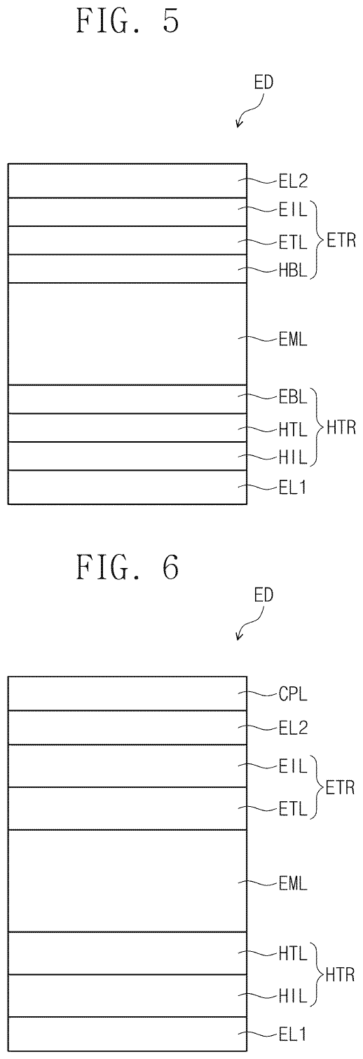

When compared to FIG. 3, FIG. 4 shows the cross-sectional view of a light emitting element ED of one or more embodiments, wherein a hole transport region HTR includes a hole injection layer HIL and a hole transport layer HTL, and an electron transport region ETR includes an electron injection layer EIL and an electron transport layer ETL. In some embodiments, when compared to FIG. 3, FIG. 5 shows the cross-sectional view of a light emitting element ED of one or more embodiments, wherein a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. When compared to FIG. 4, FIG. 6 shows the cross-sectional view of a light emitting element ED of one or more embodiments, including a capping layer CPL provided on the second electrode EL2.

The light emitting element ED of one or more embodiments may include an amine compound of one or more embodiments, as described elsewhere herein, in a hole transport region HTR. The light emitting element ED of one or more embodiments may include an amine compound of one or more embodiments in at least one among the hole injection layer HIL, hole transport layer HTL and electron blocking layer EBL of the hole transport region HTR. For example, in the light emitting element ED of one or more embodiments, the hole transport layer HTL may include the amine compound of one or more embodiments.

In the light emitting element ED according to one or more embodiments, the first electrode EL1 has conductivity (e.g., is a conductor). The first electrode EL1 may be formed utilizing a metal material, a metal alloy or a conductive compound. The first electrode EL1 may be an anode or a cathode. However, one or more embodiments of the present disclosure is not limited thereto. In some embodiments, the first electrode EL1 may be a pixel electrode. The first electrode EL1 may be a transmissive electrode, a transflective electrode, or a reflective electrode. The first electrode EL1 may include at least one selected from among Ag, Mg, Cu, Al, Pt, Pd, Au, Ni, Nd, Ir, Cr, Li, Ca, LiF, Mo, Ti, W, In, Sn, Zn, compounds of two or more selected therefrom, mixtures of two or more selected therefrom, and/or oxides thereof.

When the first electrode EL1 is the transmissive electrode, the first electrode EL1 may include a transparent metal oxide such as indium tin oxide (ITO), indium zinc oxide (IZO), zinc oxide (ZnO), and/or indium tin zinc oxide (ITZO). When the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, compound(s) thereof, or mixture(s) thereof (for example, a mixture of Ag and Mg). In one or more embodiments, the first electrode EL1 may have a structure including multiple layers including a reflective layer or a transflective layer formed utilizing the above materials, and a transmissive conductive layer formed utilizing ITO, IZO, ZnO, or ITZO. For example, the first electrode EL1 may have a three-layer structure of ITO/Ag/ITO. However, one or more embodiments of the present disclosure is not limited thereto. The first electrode EL1 may include the described metal materials, combinations of two or more metal materials selected from the described metal materials, or oxides of the described metal materials. The thickness of the first electrode EL1 may be from about 700 angstrom (Å) to about 10,000 Å. For example, the thickness of the first electrode EL1 may be from about 1,000 Å to about 3,000 Å.

The hole transport region HTR is provided on the first electrode EL1. The hole transport region HTR may have a single layer formed utilizing a single material, a single layer formed utilizing multiple different materials, or a multilayer structure including multiple layers formed utilizing multiple different materials.

The hole transport region HTR may include at least one of a hole injection layer HIL, a hole transport layer HTL, or an electron blocking layer EBL. In some embodiments, the hole transport region HTR may include multiple hole transport layers that are stacked.

In some embodiments, the hole transport region HTR may have the structure of a single layer of a hole injection layer HIL or a hole transport layer HTL, and may have a structure of a single layer formed utilizing a hole injection material and a hole transport material. In one or more embodiments, the hole transport region HTR may have a structure of a single layer formed utilizing multiple different materials, or a structure stacked from the first electrode EL1 of hole injection layer HIL/hole transport layer HTL, hole injection layer HIL/hole transport layer HTL/buffer layer, hole injection layer HIL/buffer layer, or hole transport layer HTL/buffer layer, and/or the like.

The thickness of the hole transport region HTR may be, for example, about 50 Å to about 15,000 Å. The hole transport region HTR may be formed utilizing one or more suitable methods such as a vacuum deposition method, a spin coating method, a cast method, a Langmuir-Blodgett (LB) method, an inkjet printing method, a laser printing method, and/or a laser induced thermal imaging (LITI) method.

The light emitting element ED of one or more embodiments may include the amine compound of one or more embodiments in a hole transport region HTR. In the light emitting element ED of one or more embodiments, the hole transport region HTR may include an electron injection layer EIL, and a hole transport layer HTL, and may include the amine compound of one or more embodiments in at least one among the hole injection layer EIL and the hole transport layer HTL. For example, the hole transport layer HTL may include the amine compound of one or more embodiments.

### Amine Compound

The amine compound of one or more embodiments includes a structure in which a first substituent, a second substituent and a third substituent are connected to a core nitrogen atom, (e.g., with the nitrogen atom of an amine). In the amine compound of one or more embodiments, each of the first substituent to the third substituent may be directly or indirectly combined to the core nitrogen atom, (e.g., with the nitrogen atom of the amine).

The first substituent may be a substituted or unsubstituted benzo[b]naphtho[2,1-d]thiophene group directly connected to the core nitrogen atom, (e.g., with the nitrogen atom of the amine). The first substituent may be connected to the core nitrogen atom, (e.g., with the nitrogen atom of the amine) at the carbon position 10 of the first substituent. For example, the first substituent may be represented by Formula a.

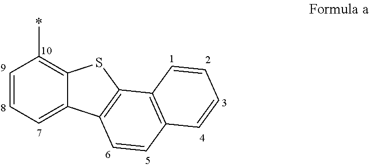

In Formula a, “* -” is a position connected to the core nitrogen atom, (e.g., with the nitrogen atom of the amine). In some embodiments, in Formula a, substituents substitutable at Formula a are not designated, and only the numbers of carbon atoms are designated, for the convenience of explanation. Hereinafter, the first substituent may be referred to as a benzonaphthothiophene group.

The second substituent may be a substituted or unsubstituted dibenzoheterole group, or a substituted or unsubstituted fluorenyl group, directly connected to the core nitrogen atom, (e.g., with the nitrogen atom of the amine). For example, the second substituent may be a substituted or unsubstituted dibenzofuran group, a substituted or unsubstituted dibenzothiophene group, or a substituted or unsubstituted fluorenyl group.

The third substituent may be a substituted or unsubstituted aryl group, or a substituted or unsubstituted heteroaryl group. The third substituent may be directly connected or indirectly connected via a linker to the core nitrogen atom, (e.g., with the nitrogen atom of the amine). In one or more embodiments, the linker connecting the core nitrogen atom (e.g., the nitrogen atom of the amine) with the third substituent may be a substituted or unsubstituted arylene group of 6 to 30 ring-forming carbon atoms, or a substituted or unsubstituted heteroarylene group of 5 to 30 ring-forming carbon atoms.

The amine compound of one or more embodiments may be a monoamine compound including a singular amine group. The amine compound of one or more embodiments may be a monoamine compound in which only one amine group is present in a state of not forming a ring in a molecular structure.

In one or more embodiments, the amine compound may be represented by Formula 1. In Formula 1, a benzonaphthothiophene group substituted with substituents R₁ and R₂ may correspond to the first substituent. In some embodiments, Ar₁ may correspond to the second substituent, and Ar₂ may correspond to the third substituent. In some embodiments, “N” in Formula 1 may correspond to the core nitrogen atom.

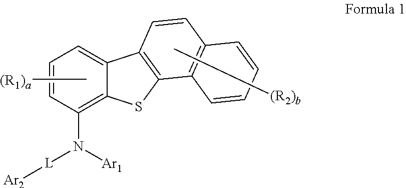

In Formula 1, R₁ and R₂ may each independently be a hydrogen atom, a deuterium atom, a substituted or unsubstituted alkyl group of 1 to 10 carbon atoms, a substituted or unsubstituted aryl group of 6 to 30 ring-forming carbon atoms, or a substituted or unsubstituted heteroaryl group of 5 to 30 ring-forming carbon atoms. For example, R₁ and R₂ may each independently be a hydrogen atom or a deuterium atom.

In Formula 1, “a” may be an integer of 0 to 3, and “b” may be an integer of 0 to 6. In Formula 1, when “a” and “b” are 0, Formula 1 may be unsubstituted with R₁ and R₂, respectively. For example, in Formula 1, when “a” is 0, the amine compound of one or more embodiments may include the first substituent which is unsubstituted with R₁. In some embodiments, when “b” is 0, the amine compound of one or more embodiments may include the first substituent which is unsubstituted with R₂. In Formula 1, a case where “a” is 3, and three R₁ are all hydrogen atoms, may be the same as a case where “a” is 0. When “a” is an integer of 2 or more, multiple R₁ may be all the same, or at least one among multiple R₁ may be different from the remainder. A case where “b” is 6, and six R₂ are all hydrogen atoms, may be the same as a case where “b” is 0. When “b” is an integer of 2 or more, multiple R₂ may be all the same, or at least one among multiple R₂ may be different from the remainder.

In Formula 1, Ar₁ may be represented by Formula 2, as described elsewhere herein.

In Formula 1, L may be a direct linkage, a substituted or unsubstituted arylene group of 6 to 30 ring-forming carbon atoms, or a substituted or unsubstituted heteroarylene group of 5 to 30 ring-forming carbon atoms. For example, L may be a direct linkage. When L is a direct linkage, Ar₂ may be directly connected to the core nitrogen atom, (e.g., with the nitrogen atom of the amine). In some embodiments, L may be a substituted or unsubstituted phenylene group, a substituted or unsubstituted biphenylene group, or a substituted or unsubstituted divalent naphthyl group. When L is substituted with another substituent, L may be substituted with a deuterium atom or an unsubstituted phenyl group, but one or more embodiments of the present disclosure is not limited thereto.

In Formula 1, Ar₂ may be a substituted or unsubstituted aryl group of 6 to 30 ring-forming carbon atoms, or a substituted or unsubstituted heteroaryl group of 5 to 30 ring-forming carbon atoms. For example, Ar₂ may be a substituted or unsubstituted phenyl group, a substituted or unsubstituted biphenyl group, a substituted or unsubstituted terphenyl group, a substituted or unsubstituted quaterphenyl group, a substituted or unsubstituted naphthyl group, a substituted or unsubstituted phenanthrenyl group, a substituted or unsubstituted dibenzofuran group, or a substituted or unsubstituted fluorenyl group. When Ar₂ is substituted with another substituent, Ar₂ may be substituted with a substituent such as a deuterium atom, an unsubstituted phenyl group, an unsubstituted biphenyl group, and an unsubstituted terphenyl group, but one or more embodiments of the present disclosure is not limited thereto.

In one or more embodiments, the amine compound represented by Formula 1 may not include (e.g., may exclude) a (e.g., any) substituted or unsubstituted carbazole group. For example, a case where Are is a substituted or unsubstituted carbazole group, may be excluded from Formula 1, (i.e., Ar₂ may not be a substituted or unsubstituted carbazole group). “-*” refers to a position to be connected.

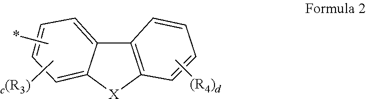

In Formula 2, X may be O, S, or CR₅R₆. For example, the amine compound represented by Formula 1 may include a dibenzofuran moiety, a dibenzothiophene moiety or a fluorene moiety, as the second substituent.

In one or more embodiments, when X is O or S and combined at an ortho position or a para position with respect to the core nitrogen atom (e.g., the nitrogen atom of the amine), a case where -L-Ar₂ of Formula 1 is an unsubstituted phenyl group, an unsubstituted biphenyl group, an unsubstituted terphenyl group, or an unsubstituted 2-phenanthrenyl group, may be excluded. In other words, when X is O or S and X is at an ortho position or a para position with respect to “N” in Formula 1, -L-Ar₂ of Formula 1 may not be an unsubstituted phenyl group, an unsubstituted biphenyl group, an unsubstituted terphenyl group, or an unsubstituted 2-phenanthrenyl group.

In Formula 2, R₃ and R₄ may each independently be a hydrogen atom, a deuterium atom, a substituted or unsubstituted alkyl group of 1 to 10 carbon atoms, a substituted or unsubstituted aryl group of 6 to 30 ring-forming carbon atoms, or a substituted or unsubstituted heteroaryl group of 5 to 30 ring-forming carbon atoms. For example, R₃ and R₄ may each independently be a hydrogen atom or a deuterium atom.

In some embodiments, the amine compound of the present disclosure may not include (e.g., may exclude) a (e.g., any) substituted or unsubstituted 9-fluorenyl group and a substituted or unsubstituted phenazine group in a molecule. For example, a case where each of R₃ and R₄ includes a substituted or unsubstituted 9-fluorenyl group, and a substituted or unsubstituted phenazine group, may be excluded, e.g., each of R₃ and R₄ may not be a substituted or unsubstituted 9-fluorenyl group, or a substituted or unsubstituted phenazine group.

In Formula 2, “c” may be an integer of 0 to 3, and “d” may be an integer of 0 to 4. In Formula 2, when “c” and “d” are 0, Formula 2 may be unsubstituted with (i.e., may not include) R₃ and R₄, respectively. For example, in Formula 2, when “c” is 0, the amine compound of one or more embodiments may include a second substituent which is unsubstituted with (i.e., does not include) R₃. In some embodiments, when “d” is 0, the amine compound of one or more embodiments may include a second substituent which is unsubstituted with (i.e., does not include) R₄. In Formula 2, a case where “c” is 3, and three R₃ are all hydrogen atoms, may be the same as a case where “c” is 0. When “c” is an integer of 2 or more, multiple R₃ may be all the same, or at least one among multiple R₃ may be different from the remainder. A case where “d” is 4, and four R₄ are all hydrogen atoms, may be the same as a case where “d” is 0. When “d” is an integer of 2 or more, multiple R₄ may be all the same, or at least one among multiple R₄ may be different from the remainder.

In Formula 2, R₅ and R₆ may each independently be a substituted or unsubstituted aryl group of 6 to 30 ring-forming carbon atoms, or a substituted or unsubstituted heteroaryl group of 5 to 30 ring-forming carbon atoms. For example, R₅ and R₆ may each independently be a substituted or unsubstituted phenyl group. When each of R₅ and R₆ is substituted, R₅ and R₆ may be substituted with a deuterium atom, but one or more embodiments of the present disclosure is not limited thereto.

In one or more embodiments, the Formula 2 may be represented by any one selected from among Substituent Group 1. For example, Ar₁ of Formula 1 may be represented by any one selected from among Substituent Group 1. In Substituent Group 1, “ [symbol] ” is a position connected to the core nitrogen atom, (e.g., with the nitrogen atom of the amine).

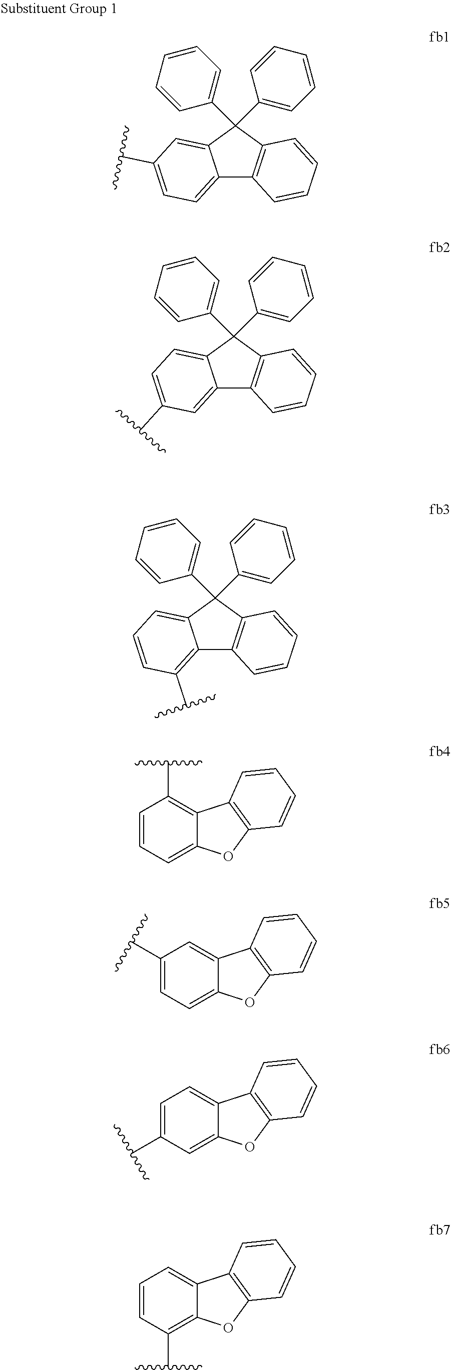

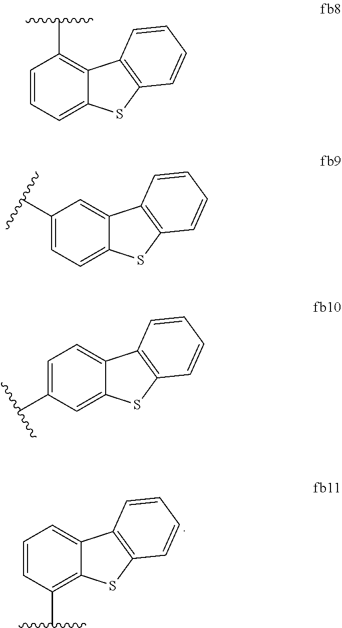 [SMILES: *c1cccc2sc3ccccc3c12 ; *c1ccc2sc3ccccc3c2c1 ; *c1ccc2c(c1)sc1ccccc12 ; *c1cccc2c1sc1ccccc12]

In one or more embodiments, -L-Ar₂ of Formula 1 may be represented by any one selected from among Substituent Group 2. In Substituent Group 2, “-*” is a position connected to the core nitrogen atom, (e.g., with the nitrogen atom of the amine).

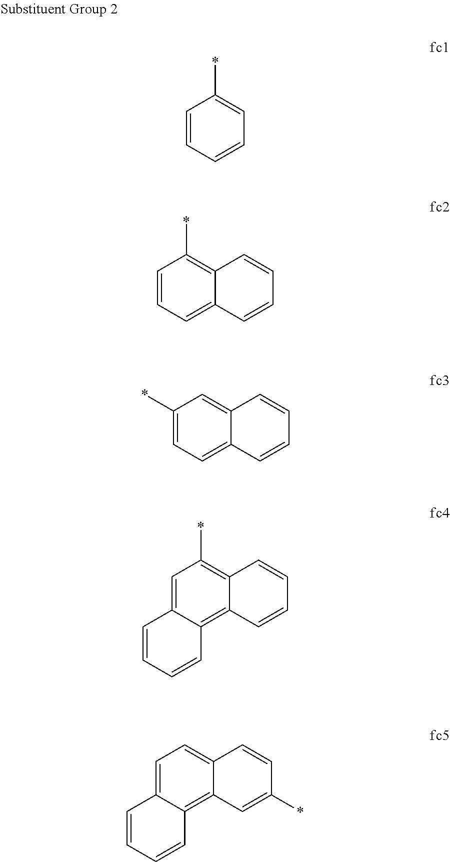 [SMILES: *c1ccccc1 ; *c1cccc2ccccc12 ; *c1ccc2ccccc2c1 ; *c1cc2ccccc2c2ccccc12 ; *c1ccc2ccc3ccccc3c2c1]

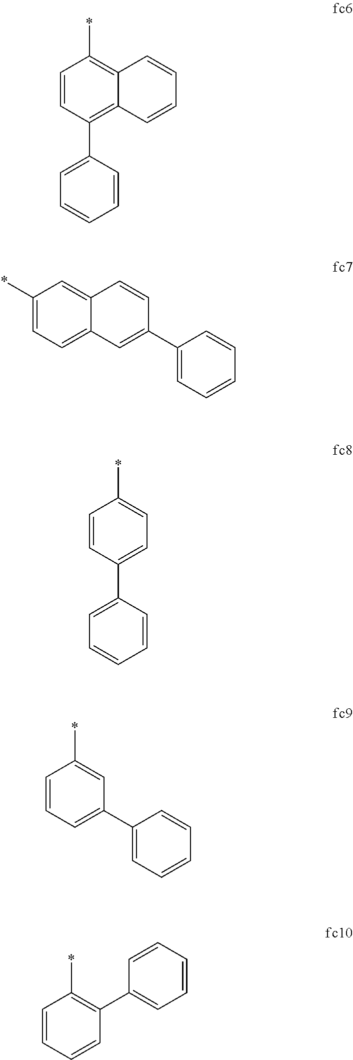 [SMILES: *c1ccc(-c2ccccc2)c2ccccc12 ; *c1ccc2cc(-c3ccccc3)ccc2c1 ; *c1ccc(-c2ccccc2)cc1 ; *c1cccc(-c2ccccc2)c1 ; *c1ccccc1-c1ccccc1]

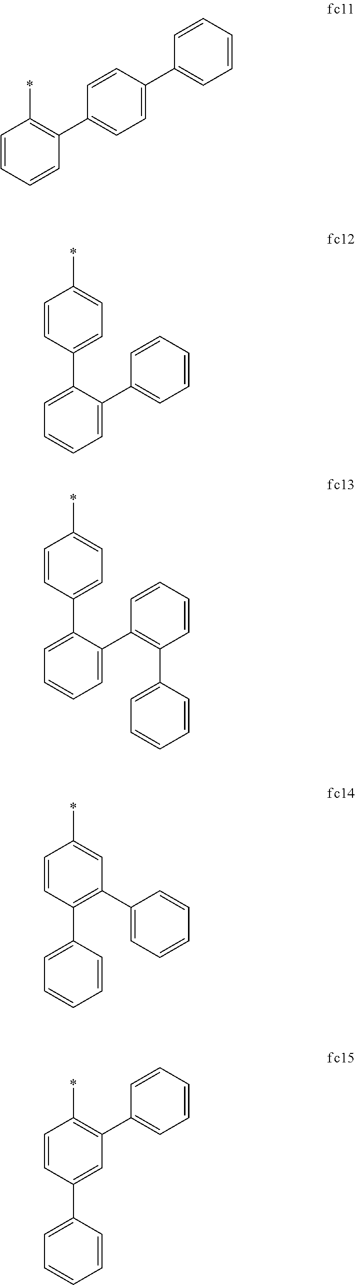

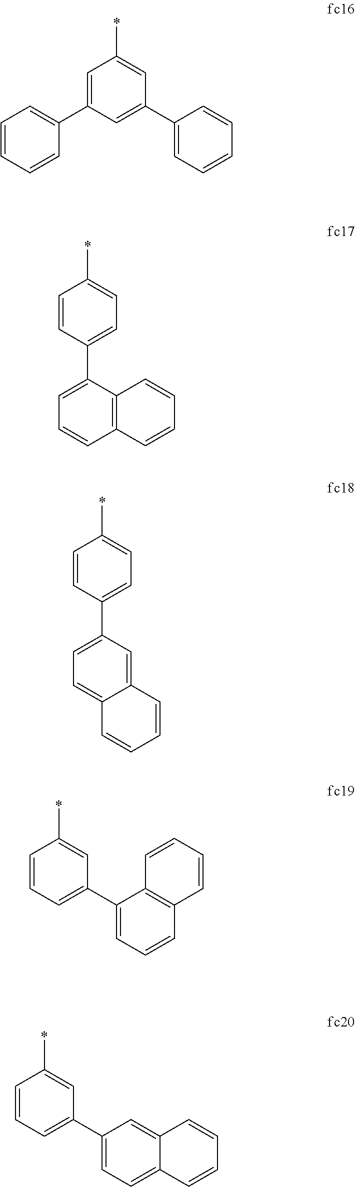 [SMILES: *c1cc(-c2ccccc2)cc(-c2ccccc2)c1 ; *c1ccc(-c2cccc3ccccc23)cc1 ; *c1ccc(-c2ccc3ccccc3c2)cc1 ; *c1cccc(-c2cccc3ccccc23)c1 ; *c1cccc(-c2ccc3ccccc3c2)c1]

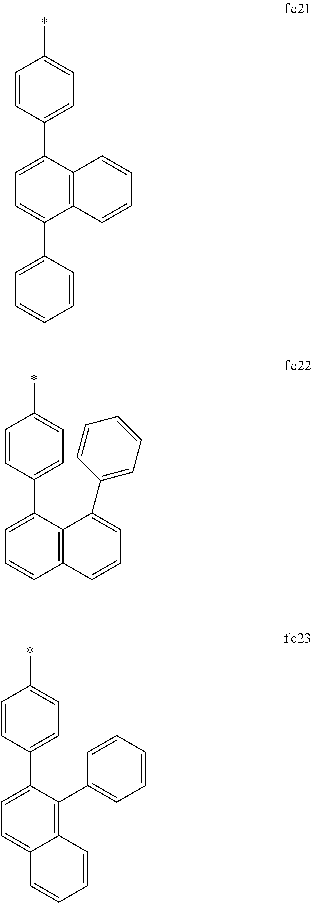 [SMILES: *c1ccc(-c2ccc(-c3ccccc3)c3ccccc23)cc1 ; *c1ccc(-c2cccc3cccc(-c4ccccc4)c23)cc1 ; *c1ccc(-c2ccc3ccccc3c2-c2ccccc2)cc1]

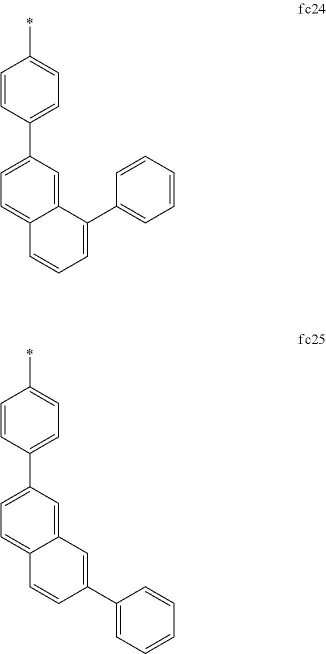 [SMILES: *c1ccc(-c2ccc3cccc(-c4ccccc4)c3c2)cc1 ; *c1ccc(-c2ccc3ccc(-c4ccccc4)cc3c2)cc1]

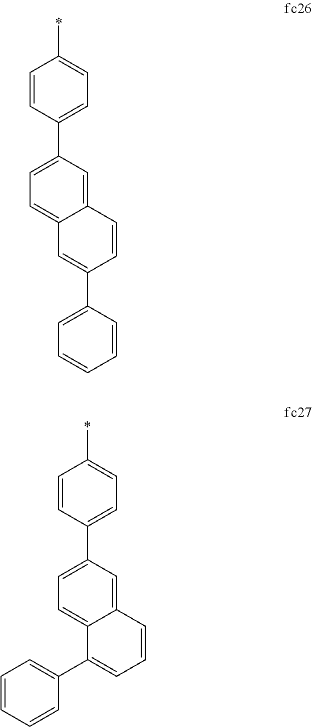 [SMILES: *c1ccc(-c2ccc3cc(-c4ccccc4)ccc3c2)cc1 ; *c1ccc(-c2ccc3c(-c4ccccc4)cccc3c2)cc1]

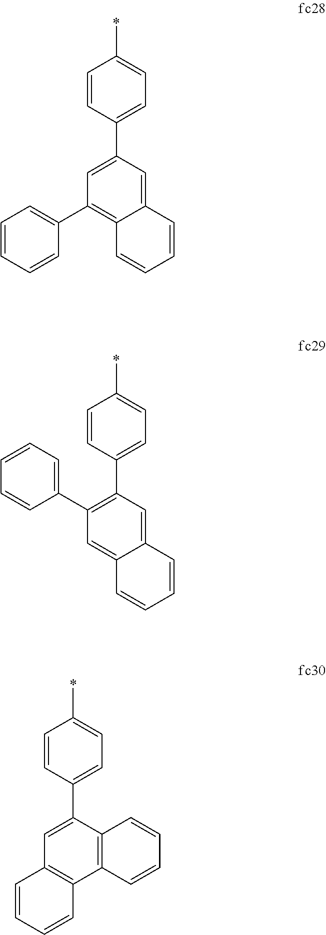 [SMILES: *c1ccc(-c2cc(-c3ccccc3)c3ccccc3c2)cc1 ; *c1ccc(-c2cc3ccccc3cc2-c2ccccc2)cc1 ; *c1ccc(-c2cc3ccccc3c3ccccc23)cc1]

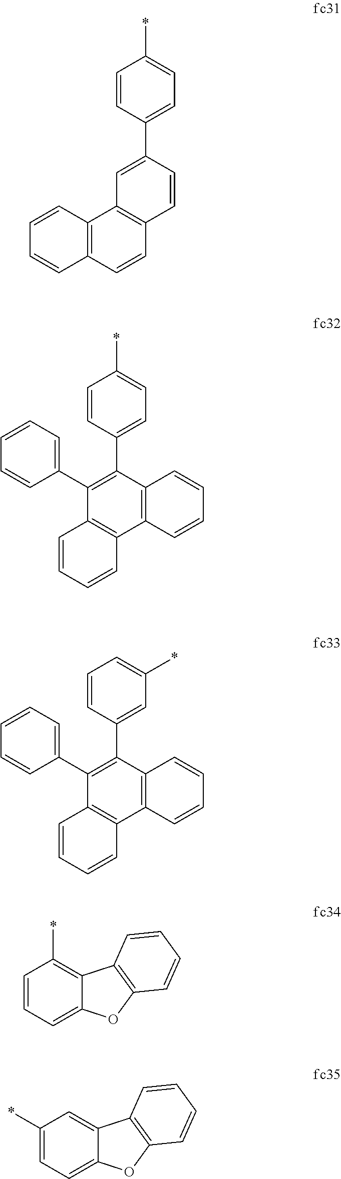 [SMILES: *c1ccc(-c2ccc3ccc4ccccc4c3c2)cc1 ; *c1ccc(-c2c(-c3ccccc3)c3ccccc3c3ccccc23)cc1 ; *c1cccc(-c2c(-c3ccccc3)c3ccccc3c3ccccc23)c1 ; *c1cccc2oc3ccccc3c12 ; *c1ccc2oc3ccccc3c2c1]

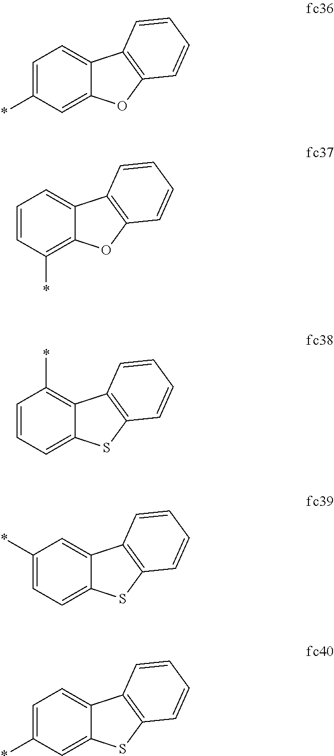 [SMILES: *c1ccc2c(c1)oc1ccccc12 ; *c1cccc2c1oc1ccccc12 ; *c1cccc2sc3ccccc3c12 ; *c1ccc2sc3ccccc3c2c1 ; *c1ccc2c(c1)sc1ccccc12]

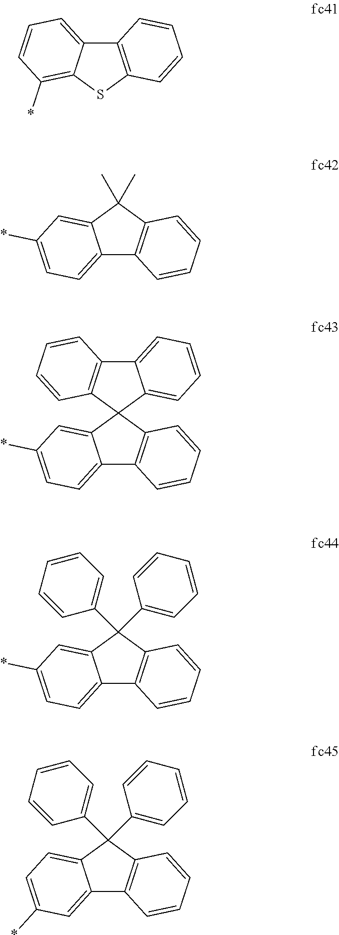 [SMILES: *c1cccc2c1sc1ccccc12 ; *c1ccc2c(c1)C(C)(C)c1ccccc1-2 ; *c1ccc2c(c1)C1(c3ccccc3-c3ccccc31)c1ccccc1-2 ; *c1ccc2c(c1)C(c1ccccc1)(c1ccccc1)c1ccccc1-2 ; *c1ccc2c(c1)-c1ccccc1C2(c1ccccc1)c1ccccc1]

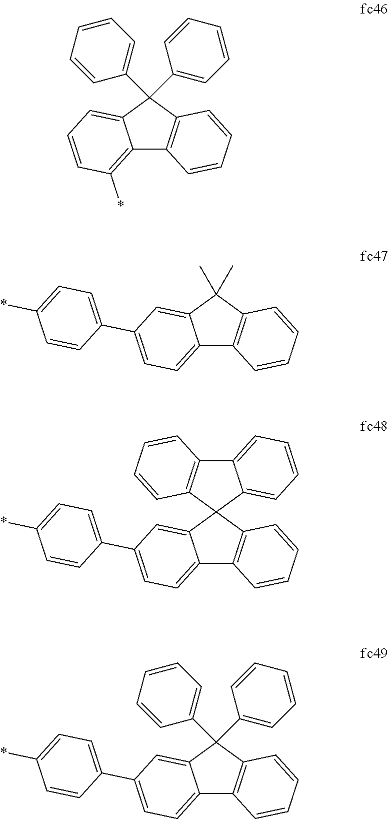 [SMILES: *c1cccc2c1-c1ccccc1C2(c1ccccc1)c1ccccc1 ; *c1ccc(-c2ccc3c(c2)C(C)(C)c2ccccc2-3)cc1 ; *c1ccc(-c2ccc3c(c2)C2(c4ccccc4-c4ccccc42)c2ccccc2-3)cc1 ; *c1ccc(-c2ccc3c(c2)C(c2ccccc2)(c2ccccc2)c2ccccc2-3)cc1]

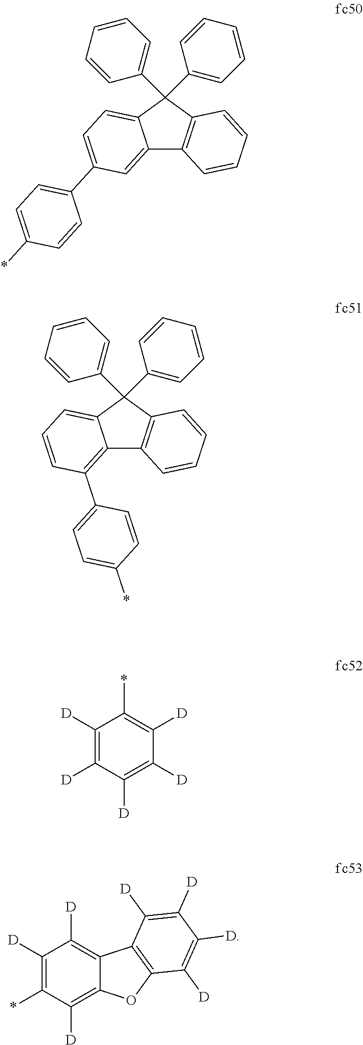

In the present disclosure, the amine compound represented by Formula 1 includes a structure in which an optional hydrogen atom is substituted with a deuterium atom. At least one among R₁, R₂, L, Ar₁, and Ar₂ in Formula 1 may include a deuterium atom, or a substituent including a deuterium atom. Formula 1 may be a structure not including a deuterium atom, and may be a structure in which partial or all hydrogen atoms are substituted with deuterium atoms.

In one or more embodiments, the amine compound represented by Formula 1 may be represented by any one among Formula 3-1 to Formula 3-3. Formula 3-1 illustrates a case where L in Formula 1 is a direct linkage, and Ar₂ is directly connected with the nitrogen atom. Formula 3-2 and Formula 3-3 illustrate cases where Are in Formula 1 is connected to the core nitrogen atom, (e.g., with the nitrogen atom of the amine) via an arylene linker.

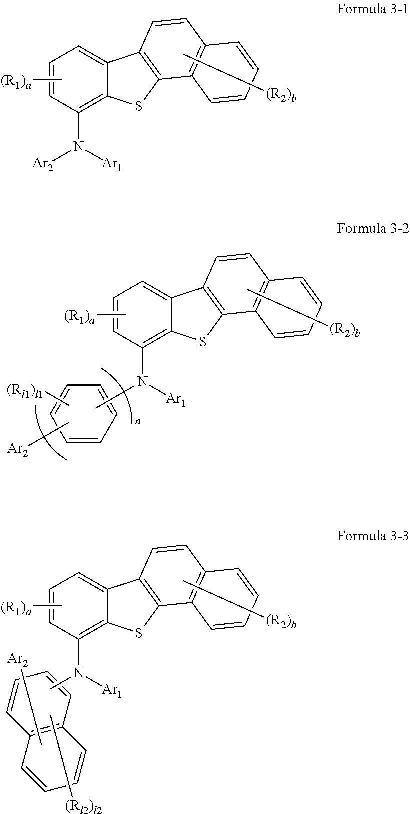

In Formula 3-1 to Formula 3-3, the same contents explained in Formula 1 may be applied for R₁, R₂, Ar₁, Ar₂, “a” and “b” each independently.

In Formula 3-2 and Formula 3-3, R_{I1} and R_{I2} may each independently be a hydrogen atom, a deuterium atom, a substituted or unsubstituted alkyl group of 1 to 10 carbon atoms, a substituted or unsubstituted aryl group of 6 to 30 ring-forming carbon atoms, or a substituted or unsubstituted heteroaryl group of 5 to 30 ring-forming carbon atoms. For example, R_{I1} and R_{I2} may each independently be a hydrogen atom, a deuterium atom, or a substituted or unsubstituted phenyl group. When R_{I1} and R_{I2} are substituted, they may be substituted with deuterium atoms, but one or more embodiments of the present disclosure is not limited thereto.

In Formula 3-2 and Formula 3-3, I1 may be an integer of 0 to 4, and I2 may be an integer of 0 to 6. In Formula 3-2 and Formula 3-3, when I1 and I2 are 0, the amine compound of one or more embodiments may be unsubstituted with R_{I1} and R_{I2}, respectively. A case where I1 is 4, and four R_{I1} are all hydrogen atoms, may be the same as a case where I1 is 0. A case where I2 is 6, and six R_{I2} are all hydrogen atoms, may be the same as a case where I2 is 0. When I1 and I2 are integers of 2 or more, each of multiple R_{I1} and R_{I2} may be all the same, or at least one among each of multiple R_{I1} and R_{I2} may be different from the remainder.

In Formula 3-2, “n” may be an integer of 1 to 3. When “n” is 1, Ar₂ may be substituted with R_{I1}, or connected to the core nitrogen atom, (e.g., with the nitrogen atom of the amine) via an unsubstituted phenylene group as a linker. When “n” is 2, Ar₂ may be substituted with R_{I1}, or connected to the core nitrogen atom, (e.g., with the nitrogen atom of the amine) via an unsubstituted biphenylene group as a linker. When “n” is 3, Ar₂ may be substituted with R_{I1}, or connected to the core nitrogen atom, (e.g., with the nitrogen atom of the amine) via an unsubstituted terphenylene group as a linker.

In one or more embodiments, the amine compound represented by Formula 1 may be represented by any one among (e.g., one selected from among) Formula 4-1 to Formula 4-3. Formula 4-1 illustrates a case of Formula 1 where Ar₁ is represented by Formula 2, and X is O. Formula 4-2 illustrates a case of Formula 1 where Ar₁ is represented by Formula 2, and X is S. Formula 4-3 illustrates a case of Formula 1 where Ar₁ is represented by Formula 2, and X is CR₅R₆, where R₅ and R₆ are substituted or unsubstituted phenyl groups. In Formula 4-1 to Formula 4-3, the same contents explained in Formula 1 may be applied for Ar₂ and L.

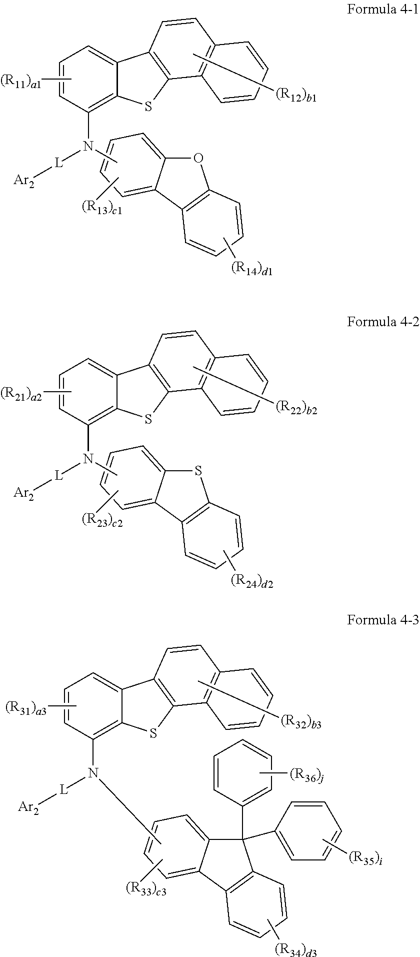

In Formula 4-1 to Formula 4-3, R₁₁ to R₁₄, R₂₁ to R₂₄, and R₃₁ to R₃₆ may each independently be a hydrogen atom or a deuterium atom. In Formula 4-1 to Formula 4-3, a1 to a3, and c1 to c3 may each independently be an integer of 0 to 3. In Formula 4-1 to Formula 4-3, b1 to b3 may each independently be an integer of 0 to 6. d1 to d3 may each independently be an integer of 0 to 4. In Formula 4-1 to Formula 4-3, “i” and “j” may each independently be an integer of 0 to 5.

In one or more embodiments, when a1 to a3, b1 to b3, c1 to c3, and d1 to d3 are 0, the amine compound of one or more embodiments may be unsubstituted with R₁₁ to R₁₄, R₂₁ to R₂₄, and R₃₁ to R₃₆.

For example, cases where a1 to a3, and c1 to c3 are 3, and three R₁₁, R₂₁, R₃₁, R₁₃, R₂₃, and R₃₃ are all hydrogen atoms, may be the same as cases where a1 to a3, and c1 to c3 are 0, respectively. When each of a1 to a3, and c1 to c3 are 3 is an integer of 2 or more, each of multiple R₁₁, R₂₁, R₃₁, R₁₃, R₂₃, and R₃₃ may be all the same, or at least one among each of multiple R₁₁, R₂₁, R₃₁, R₁₃, R₂₃, and R₃₃ may be different from the remainder.

For example, a case where each of b1 to b3 is 6, and each of six R₁₂, R₂₂, and R₃₂ are hydrogen atoms, may be the same as cases where each of b1 to b3 are 0, respectively. When each of b1 to b3 is an integer of 2 or more, each of multiple R₁₂, R₂₂, and R₃₂ may be all the same, or at least one among each of multiple R₁₂, R₂₂, and R₃₂ may be different from the remainder.

For example, a case where each of d1 to d3 is 4, and each of four R₁₄, R₂₄, and R₃₄ are hydrogen atoms, may be the same as cases where each of d1 to d3 are 0, respectively. When each of d1 to d3 is an integer of 2 or more, each of multiple R₁₄, R₂₄, and R₃₄ may be all the same, or at least one among each of multiple R₁₄, R₂₄, and R₃₄ may be different from the remainder.

For example, a case where each of “i” and “j” is 5, and each of five R₃₅, and R₃₆ are hydrogen atoms, may be the same as cases where each of “i” and “j” are 0, respectively. When each of “i” and “j” is an integer of 2 or more, each of multiple R₃₅, and R₃₆ may be all the same, or at least one among each of multiple R₃₅, and R₃₆ may be different from the remainder.

In one or more embodiments, the amine compound represented by Formula 1 may be represented by Formula 5-1 or Formula 5-2. Formula 5-1 and Formula 5-2 represent cases of Formula 1 where Ar₂ is specified. In Formula 5-1 and Formula 5-2, the same contents explained in Formula 1 may be applied for R₁, R₂, L, Ar₁, “a” and “b”.

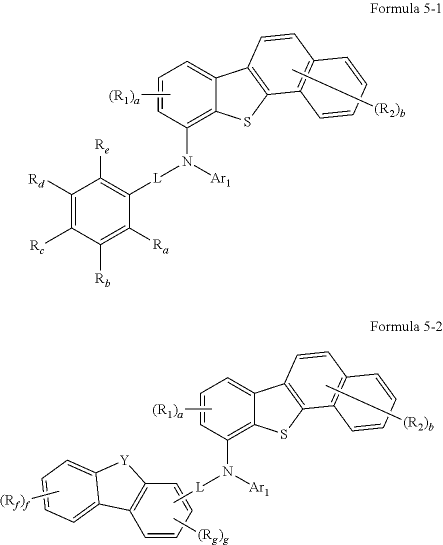

In Formula 5-1, Rₐ to Rₑ may each independently be a hydrogen atom, a deuterium atom, or a substituted or unsubstituted aryl group of 6 to 20 ring-forming carbon atoms. In some embodiments, a part (i.e., atom(s) or element(s)) of at least one pair selected from among Rₐ and R_{b}, R_{b} and R_{c}, R_{c} and R_{d}, and/or R_{d} and Rₑ may form, or may be combined with each other to form, a substituted or unsubstituted aromatic hydrocarbon ring. For example, Rₐ to Rₑ may each independently be a hydrogen atom, a deuterium atom, a substituted or unsubstituted phenyl group, a substituted or unsubstituted biphenyl group, or a substituted or unsubstituted terphenyl group. In some embodiments, a part (i.e., atom(s) or element(s)) of any one pair selected from among Rₐ and R_{b}, R_{b} and R_{c}, R_{c} and R_{d}, and/or Rₐ and Rₑ may form, or may be combined with each other to form, a substituted or unsubstituted aromatic hydrocarbon ring. When a part (i.e., atom(s) or element(s)) of any one pair selected from among Rₐ and R_{b}, R_{b} and R_{c}, R_{c} and R_{d}, and/or R_{d} and Rₑ are combined with each other, a benzene ring in which Rₐ to Rₑ are connected, may be a substituted or unsubstituted naphthyl group. In some embodiments, a part (i.e., atom(s) or element(s)) of any two pairs selected from among Rₐ and R_{b}, R_{b} and R_{c}, R_{c} and R_{d}, and/or R_{d} and Rₑ may be combined with each other to form substituted or unsubstituted aromatic hydrocarbon rings. When a part (i.e., atom(s) or element(s)) of any two pairs selected from among Rₐ and R_{b}, R_{b} and R_{c}, R_{c} and R_{d}, and/or R_{d} and Rₑ are combined with each other, a benzene ring in which Rₐ to Rₑ are connected, may be a substituted or unsubstituted phenanthrenyl group.

In Formula 5-2, Y may be O, S or CR_{y1}R_{y2}. For example, the third substituent may be a substituted or unsubstituted dibenzofuran group, a substituted or unsubstituted dibenzothiophene group, or a substituted or unsubstituted fluorenyl group.

In Formula 5-2, R_{f} and R_{g} may each independently be a hydrogen atom or a deuterium atom. In some embodiments, “f” may be an integer of 0 to 4, and “g” may be an integer of 0 to 3. In Formula 5-2, when “f” and “g” are 0, the amine compound of one or more embodiments may be unsubstituted with R_{f} and R_{g}, respectively. A case where “f” is 4, and four R_{f} are hydrogen atoms, may be the same as a case where “f” is 0. In some embodiments, a case where “g” is 3, and three R_{g} are hydrogen atoms, may be the same as a case where “g” is 0. When each of “f” and “g” is an integer of 2 or more, each of multiple R_{f} and R_{g} may be all the same, or at least one among each of multiple R_{f} and R_{g} may different from the remainder.

In Formula 2, R_{y1} and R_{y2} may each independently be a substituted or unsubstituted alkyl group of 1 to 10 carbon atoms, or a substituted or unsubstituted aryl group of 6 to 15 ring-forming carbon atoms. In some embodiments, R_{y1} and R_{y2} may form, or may be combined with each other to form, a ring. For example, R_{y1} and R_{y2} may each independently be a substituted or unsubstituted methyl group, or a substituted or unsubstituted phenyl group. In some embodiments, R_{y1} and R_{y2} may form, or may be combined with each other to form, a spiro structure. When R_{y1} and R_{y2} are substituted, R_{y1} and R_{y2} may be substituted with deuterium atoms, but one or more embodiments of the present disclosure is not limited thereto.

In one or more embodiments, the amine compound represented by Formula 1 may be represented by Formula 1-a. In Formula 1-a, Fa may correspond to the first substituent, and Fb may correspond to the second substituent. Fc may include the third substituent, and the linker between the third substituent and the core nitrogen atom (e.g., the nitrogen atom of the amine). The amine compound of one or more embodiments, represented by Formula 1-a may be a compound satisfying any one among the combinations represented in Compound Combination Table 1.

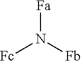

In Formula 1-a, Fa may be represented by Substituent fa1. In Substituent fa1, “ [symbol] ” is a position connected to the core nitrogen atom, (e.g., with the nitrogen atom of the amine).

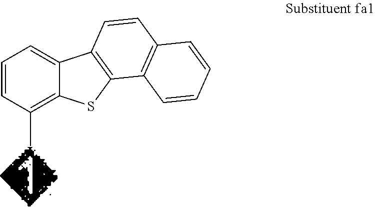 [SMILES: Cc1cccc2c1sc1c3ccccc3ccc21]

In Formula 1-a, Fb may be selected from Substituent Group 1 above, and Fc may be selected from Substituent Group 2 above.

Compound Combination Table 1

| Entry | Fa | Fb | Fc |
|---|---|---|---|
| A1 | fa1 | fb1 | fc1 |
| A2 | fa1 | fb1 | fc2 |
| A3 | fa1 | fb1 | fc3 |
| A4 | fa1 | fb1 | fc4 |
| A5 | fa1 | fb1 | fc5 |
| A6 | fa1 | fb1 | fc6 |
| A7 | fa1 | fb1 | fc7 |
| A8 | fa1 | fb1 | fc8 |
| A9 | fa1 | fb1 | fc9 |
| A10 | fa1 | fb1 | fc10 |
| A11 | fa1 | fb1 | fc11 |
| A12 | fa1 | fb1 | fc12 |
| A13 | fa1 | fb1 | fc13 |
| A14 | fa1 | fb1 | fc14 |
| A15 | fa1 | fb1 | fc15 |
| A16 | fa1 | fb1 | fc16 |
| A17 | fa1 | fb1 | fc17 |
| A18 | fa1 | fb1 | fc18 |
| A19 | fa1 | fb1 | fc19 |
| A20 | fa1 | fb1 | fc20 |
| A21 | fa1 | fb1 | fc21 |
| A22 | fa1 | fb1 | fc22 |
| A23 | fa1 | fb1 | fc23 |
| A24 | fa1 | fb1 | fc24 |
| A25 | fa1 | fb1 | fc25 |
| A26 | fa1 | fb1 | fc26 |
| A27 | fa1 | fb1 | fc27 |
| A28 | fa1 | fb1 | fc28 |
| A29 | fa1 | fb1 | fc29 |
| A30 | fa1 | fb1 | fc30 |
| A31 | fa1 | fb1 | fc31 |
| A32 | fa1 | fb1 | fc32 |
| A33 | fa1 | fb1 | fc33 |
| A34 | fa1 | fb1 | fc34 |
| A35 | fa1 | fb1 | fc35 |
| A36 | fa1 | fb1 | fc36 |
| A37 | fa1 | fb1 | fc37 |
| A38 | fa1 | fb1 | fc38 |
| A39 | fa1 | fb1 | fc39 |
| A40 | fa1 | fb1 | fc40 |
| A41 | fa1 | fb1 | fc41 |
| A42 | fa1 | fb1 | fc42 |
| A43 | fa1 | fb1 | fc43 |
| A44 | fa1 | fb1 | fc44 |
| A45 | fa1 | fb1 | fc45 |
| A46 | fa1 | fb1 | fc46 |
| A47 | fa1 | fb1 | fc47 |
| A48 | fa1 | fb1 | fc48 |
| A49 | fa1 | fb1 | fc49 |
| A50 | fa1 | fb1 | fc50 |
| A51 | fa1 | fb1 | fc51 |
| A52 | fa1 | fb1 | fc52 |
| A53 | fa1 | fb1 | fc53 |
| B1 | fa1 | fb2 | fc1 |
| B2 | fa1 | fb2 | fc2 |
| B3 | fa1 | fb2 | fc3 |
| B4 | fa1 | fb2 | fc4 |
| B5 | fa1 | fb2 | fc5 |
| B6 | fa1 | fb2 | fc6 |
| B7 | fa1 | fb2 | fc7 |
| B8 | fa1 | fb2 | fc8 |
| B9 | fa1 | fb2 | fc9 |
| B10 | fa1 | fb2 | fc10 |
| B11 | fa1 | fb2 | fc11 |
| B12 | fa1 | fb2 | fc12 |
| B13 | fa1 | fb2 | fc13 |
| B14 | fa1 | fb2 | fc14 |
| B15 | fa1 | fb2 | fc15 |
| B16 | fa1 | fb2 | fc16 |
| B17 | fa1 | fb2 | fc17 |
| B18 | fa1 | fb2 | fc18 |
| B19 | fa1 | fb2 | fc19 |
| B20 | fa1 | fb2 | fc20 |
| B21 | fa1 | fb2 | fc21 |
| B22 | fa1 | fb2 | fc22 |
| B23 | fa1 | fb2 | fc23 |
| B24 | fa1 | fb2 | fc24 |
| B25 | fa1 | fb2 | fc25 |
| B26 | fa1 | fb2 | fc26 |
| B27 | fa1 | fb2 | fc27 |
| B28 | fa1 | fb2 | fc28 |
| B29 | fa1 | fb2 | fc29 |
| B30 | fa1 | fb2 | fc30 |
| B31 | fa1 | fb2 | fc31 |
| B32 | fa1 | fb2 | fc32 |
| B33 | fa1 | fb2 | fc33 |
| B34 | fa1 | fb2 | fc34 |
| B35 | fa1 | fb2 | fc35 |
| B36 | fa1 | fb2 | fc36 |
| B37 | fa1 | fb2 | fc37 |
| B38 | fa1 | fb2 | fc38 |
| B39 | fa1 | fb2 | fc39 |
| B40 | fa1 | fb2 | fc40 |
| B41 | fa1 | fb2 | fc41 |
| B42 | fa1 | fb2 | fc42 |
| B43 | fa1 | fb2 | fc43 |
| B44 | fa1 | fb2 | fc44 |
| B45 | fa1 | fb2 | fc45 |
| B46 | fa1 | fb2 | fc46 |
| B47 | fa1 | fb2 | fc47 |
| B48 | fa1 | fb2 | fc48 |
| B49 | fa1 | fb2 | fc49 |
| B50 | fa1 | fb2 | fc50 |
| B51 | fa1 | fb2 | fc51 |
| B52 | fa1 | fb2 | fc52 |
| B53 | fa1 | fb2 | fc53 |
| C1 | fa1 | fb3 | fc1 |
| C2 | fa1 | fb3 | fc2 |
| C3 | fa1 | fb3 | fc3 |
| C4 | fa1 | fb3 | fc4 |
| C5 | fa1 | fb3 | fc5 |
| C6 | fa1 | fb3 | fc6 |
| C7 | fa1 | fb3 | fc7 |
| C8 | fa1 | fb3 | fc8 |
| C9 | fa1 | fb3 | fc9 |
| C10 | fa1 | fb3 | fc10 |
| C11 | fa1 | fb3 | fc11 |
| C12 | fa1 | fb3 | fc12 |
| C13 | fa1 | fb3 | fc13 |
| C14 | fa1 | fb3 | fc14 |
| C15 | fa1 | fb3 | fc15 |
| C16 | fa1 | fb3 | fc16 |
| C17 | fa1 | fb3 | fc17 |
| C18 | fa1 | fb3 | fc18 |
| C19 | fa1 | fb3 | fc19 |
| C20 | fa1 | fb3 | fc20 |
| C21 | fa1 | fb3 | fc21 |
| C22 | fa1 | fb3 | fc22 |
| C23 | fa1 | fb3 | fc23 |
| C24 | fa1 | fb3 | fc24 |
| C25 | fa1 | fb3 | fc25 |
| C26 | fa1 | fb3 | fc26 |
| C27 | fa1 | fb3 | fc27 |
| C28 | fa1 | fb3 | fc28 |
| C29 | fa1 | fb3 | fc29 |
| C30 | fa1 | fb3 | fc30 |
| C31 | fa1 | fb3 | fc31 |
| C32 | fa1 | fb3 | fc32 |
| C33 | fa1 | fb3 | fc33 |
| C34 | fa1 | fb3 | fc34 |
| C35 | fa1 | fb3 | fc35 |
| C36 | fa1 | fb3 | fc36 |
| C37 | fa1 | fb3 | fc37 |
| C38 | fa1 | fb3 | fc38 |
| C39 | fa1 | fb3 | fc39 |
| C40 | fa1 | fb3 | fc40 |
| C41 | fa1 | fb3 | fc41 |
| C42 | fa1 | fb3 | fc42 |
| C43 | fa1 | fb3 | fc43 |
| C44 | fa1 | fb3 | fc44 |
| C45 | fa1 | fb3 | fc45 |
| C46 | fa1 | fb3 | fc46 |
| C47 | fa1 | fb3 | fc47 |
| C48 | fa1 | fb3 | fc48 |
| C49 | fa1 | fb3 | fc49 |
| C50 | fa1 | fb3 | fc50 |
| C51 | fa1 | fb3 | fc51 |
| C52 | fa1 | fb3 | fc52 |
| C53 | fa1 | fb3 | fc53 |
| D1 | fa1 | fb4 | fc1 |
| D2 | fa1 | fb4 | fc2 |
| D3 | fa1 | fb4 | fc3 |
| D4 | fa1 | fb4 | fc4 |
| D5 | fa1 | fb4 | fc5 |
| D6 | fa1 | fb4 | fc6 |
| D7 | fa1 | fb4 | fc7 |
| D8 | fa1 | fb4 | fc8 |
| D9 | fa1 | fb4 | fc9 |
| D10 | fa1 | fb4 | fc10 |
| D11 | fa1 | fb4 | fc11 |
| D12 | fa1 | fb4 | fc12 |
| D13 | fa1 | fb4 | fc13 |
| D14 | fa1 | fb4 | fc14 |
| D15 | fa1 | fb4 | fc15 |
| D16 | fa1 | fb4 | fc16 |
| D17 | fa1 | fb4 | fc17 |
| D18 | fa1 | fb4 | fc18 |
| D19 | fa1 | fb4 | fc19 |
| D20 | fa1 | fb4 | fc20 |
| D21 | fa1 | fb4 | fc21 |
| D22 | fa1 | fb4 | fc22 |
| D23 | fa1 | fb4 | fc23 |
| D24 | fa1 | fb4 | fc24 |
| D25 | fa1 | fb4 | fc25 |
| D26 | fa1 | fb4 | fc26 |
| D27 | fa1 | fb4 | fc27 |
| D28 | fa1 | fb4 | fc28 |
| D29 | fa1 | fb4 | fc29 |
| D30 | fa1 | fb4 | fc30 |
| D31 | fa1 | fb4 | fc31 |
| D32 | fa1 | fb4 | fc32 |
| D33 | fa1 | fb4 | fc33 |
| D34 | fa1 | fb4 | fc34 |
| D35 | fa1 | fb4 | fc35 |
| D36 | fa1 | fb4 | fc36 |
| D37 | fa1 | fb4 | fc37 |
| D38 | fa1 | fb4 | fc38 |
| D39 | fa1 | fb4 | fc39 |
| D40 | fa1 | fb4 | fc40 |
| D41 | fa1 | fb4 | fc41 |
| D42 | fa1 | fb4 | fc42 |
| D43 | fa1 | fb4 | fc43 |
| D44 | fa1 | fb4 | fc44 |
| D45 | fa1 | fb4 | fc45 |
| D46 | fa1 | fb4 | fc46 |
| D47 | fa1 | fb4 | fc47 |
| D48 | fa1 | fb4 | fc48 |
| D49 | fa1 | fb4 | fc49 |
| D50 | fa1 | fb4 | fc50 |
| D51 | fa1 | fb4 | fc51 |
| D52 | fa1 | fb4 | fc52 |
| D53 | fa1 | fb4 | fc53 |
| E1 | fa1 | fb5 | fc2 |
| E2 | fa1 | fb5 | fc3 |
| E3 | fa1 | fb5 | fc4 |
| E4 | fa1 | fb5 | fc6 |
| E5 | fa1 | fb5 | fc7 |
| E6 | fa1 | fb5 | fc17 |
| E7 | fa1 | fb5 | fc18 |
| E8 | fa1 | fb5 | fc19 |
| E9 | fa1 | fb5 | fc20 |
| E10 | fa1 | fb5 | fc21 |
| E11 | fa1 | fb5 | fc22 |
| E12 | fa1 | fb5 | fc23 |
| E13 | fa1 | fb5 | fc24 |
| E14 | fa1 | fb5 | fc25 |
| E15 | fa1 | fb5 | fc26 |
| E16 | fa1 | fb5 | fc27 |
| E17 | fa1 | fb5 | fc28 |
| E18 | fa1 | fb5 | fc29 |
| E19 | fa1 | fb5 | fc30 |
| E20 | fa1 | fb5 | fc31 |
| E21 | fa1 | fb5 | fc32 |
| E22 | fa1 | fb5 | fc33 |
| E23 | fa1 | fb5 | fc34 |
| E24 | fa1 | fb5 | fc35 |
| E25 | fa1 | fb5 | fc36 |
| E26 | fa1 | fb5 | fc37 |
| E27 | fa1 | fb5 | fc38 |
| E28 | fa1 | fb5 | fc39 |
| E29 | fa1 | fb5 | fc40 |
| E30 | fa1 | fb5 | fc41 |
| E31 | fa1 | fb5 | fc42 |
| E32 | fa1 | fb5 | fc43 |
| E33 | fa1 | fb5 | fc44 |
| E34 | fa1 | fb5 | fc45 |
| E35 | fa1 | fb5 | fc46 |
| E36 | fa1 | fb5 | fc47 |
| E37 | fa1 | fb5 | fc48 |
| E38 | fa1 | fb5 | fc49 |
| E39 | fa1 | fb5 | fc50 |
| E40 | fa1 | fb5 | fc51 |
| E41 | fa1 | fb5 | fc52 |
| E42 | fa1 | fb5 | fc53 |
| F1 | fa1 | fb6 | fc1 |
| F2 | fa1 | fb6 | fc2 |
| F3 | fa1 | fb6 | fc3 |
| F4 | fa1 | fb6 | fc4 |
| F5 | fa1 | fb6 | fc5 |
| F6 | fa1 | fb6 | fc6 |
| F7 | fa1 | fb6 | fc7 |
| F8 | fa1 | fb6 | fc8 |
| F9 | fa1 | fb6 | fc9 |
| F10 | fa1 | fb6 | fc10 |
| F11 | fa1 | fb6 | fc11 |
| F12 | fa1 | fb6 | fc12 |
| F13 | fa1 | fb6 | fc13 |
| F14 | fa1 | fb6 | fc14 |
| F15 | fa1 | fb6 | fc15 |
| F16 | fa1 | fb6 | fc16 |
| F17 | fa1 | fb6 | fc17 |
| F18 | fa1 | fb6 | fc18 |
| F19 | fa1 | fb6 | fc19 |
| F20 | fa1 | fb6 | fc20 |
| F21 | fa1 | fb6 | fc21 |
| F22 | fa1 | fb6 | fc22 |
| F23 | fa1 | fb6 | fc23 |
| F24 | fa1 | fb6 | fc24 |
| F25 | fa1 | fb6 | fc25 |
| F26 | fa1 | fb6 | fc26 |
| F27 | fa1 | fb6 | fc27 |
| F28 | fa1 | fb6 | fc28 |
| F29 | fa1 | fb6 | fc29 |
| F30 | fa1 | fb6 | fc30 |
| F31 | fa1 | fb6 | fc31 |
| F32 | fa1 | fb6 | fc32 |
| F33 | fa1 | fb6 | fc33 |
| F34 | fa1 | fb6 | fc34 |
| F35 | fa1 | fb6 | fc35 |
| F36 | fa1 | fb6 | fc36 |
| F37 | fa1 | fb6 | fc37 |
| F38 | fa1 | fb6 | fc38 |
| F39 | fa1 | fb6 | fc39 |
| F40 | fa1 | fb6 | fc40 |
| F41 | fa1 | fb6 | fc41 |
| F42 | fa1 | fb6 | fc42 |
| F43 | fa1 | fb6 | fc43 |
| F44 | fa1 | fb6 | fc44 |
| F45 | fa1 | fb6 | fc45 |
| F46 | fa1 | fb6 | fc46 |
| F47 | fa1 | fb6 | fc47 |
| F48 | fa1 | fb6 | fc48 |
| F49 | fa1 | fb6 | fc49 |
| F50 | fa1 | fb6 | fc50 |
| F51 | fa1 | fb6 | fc51 |
| F52 | fa1 | fb6 | fc52 |
| G1 | fa1 | fb7 | fc2 |
| G2 | fa1 | fb7 | fc3 |
| G3 | fa1 | fb7 | fc4 |
| G4 | fa1 | fb7 | fc6 |
| G5 | fa1 | fb7 | fc7 |
| G6 | fa1 | fb7 | fc17 |
| G7 | fa1 | fb7 | fc18 |
| G8 | fa1 | fb7 | fc19 |
| G9 | fa1 | fb7 | fc20 |
| G10 | fa1 | fb7 | fc21 |
| G11 | fa1 | fb7 | fc22 |
| G12 | fa1 | fb7 | fc23 |
| G13 | fa1 | fb7 | fc24 |
| G14 | fa1 | fb7 | fc25 |
| G15 | fa1 | fb7 | fc26 |
| G16 | fa1 | fb7 | fc27 |
| G17 | fa1 | fb7 | fc28 |
| G18 | fa1 | fb7 | fc29 |
| G19 | fa1 | fb7 | fc30 |
| G20 | fa1 | fb7 | fc31 |
| G21 | fa1 | fb7 | fc32 |
| G22 | fa1 | fb7 | fc33 |
| G23 | fa1 | fb7 | fc34 |
| G24 | fa1 | fb7 | fc35 |
| G25 | fa1 | fb7 | fc36 |
| G26 | fa1 | fb7 | fc37 |
| G27 | fa1 | fb7 | fc38 |
| G28 | fa1 | fb7 | fc39 |
| G29 | fa1 | fb7 | fc40 |
| G30 | fa1 | fb7 | fc41 |
| G31 | fa1 | fb7 | fc42 |
| G32 | fa1 | fb7 | fc43 |
| G33 | fa1 | fb7 | fc44 |
| G34 | fa1 | fb7 | fc45 |
| G35 | fa1 | fb7 | fc46 |
| G36 | fa1 | fb7 | fc47 |
| G37 | fa1 | fb7 | fc48 |
| G38 | fa1 | fb7 | fc49 |
| G39 | fa1 | fb7 | fc50 |
| G40 | fa1 | fb7 | fc51 |
| G41 | fa1 | fb7 | fc52 |
| G42 | fa1 | fb7 | fc53 |
| H1 | fa1 | fb8 | fc1 |
| H2 | fa1 | fb8 | fc2 |
| H3 | fa1 | fb8 | fc3 |
| H4 | fa1 | fb8 | fc4 |
| H5 | fa1 | fb8 | fc5 |
| H6 | fa1 | fb8 | fc6 |
| H7 | fa1 | fb8 | fc7 |
| H8 | fa1 | fb8 | fc8 |
| H9 | fa1 | fb8 | fc9 |
| H10 | fa1 | fb8 | fc10 |
| H11 | fa1 | fb8 | fc11 |
| H12 | fa1 | fb8 | fc12 |
| H13 | fa1 | fb8 | fc13 |
| H14 | fa1 | fb8 | fc14 |
| H15 | fa1 | fb8 | fc15 |
| H16 | fa1 | fb8 | fc16 |
| H17 | fa1 | fb8 | fc17 |
| H18 | fa1 | fb8 | fc18 |
| H19 | fa1 | fb8 | fc19 |
| H20 | fa1 | fb8 | fc20 |
| H21 | fa1 | fb8 | fc21 |
| H22 | fa1 | fb8 | fc22 |
| H23 | fa1 | fb8 | fc23 |
| H24 | fa1 | fb8 | fc24 |
| H25 | fa1 | fb8 | fc25 |
| H26 | fa1 | fb8 | fc26 |
| H27 | fa1 | fb8 | fc27 |
| H28 | fa1 | fb8 | fc28 |
| H29 | fa1 | fb8 | fc29 |
| H30 | fa1 | fb8 | fc30 |
| H31 | fa1 | fb8 | fc31 |
| H32 | fa1 | fb8 | fc32 |
| H33 | fa1 | fb8 | fc33 |
| H34 | fa1 | fb8 | fc34 |
| H35 | fa1 | fb8 | fc35 |
| H36 | fa1 | fb8 | fc36 |
| H37 | fa1 | fb8 | fc37 |
| H38 | fa1 | fb8 | fc38 |
| H39 | fa1 | fb8 | fc39 |
| H40 | fa1 | fb8 | fc40 |
| H41 | fa1 | fb8 | fc41 |
| H42 | fa1 | fb8 | fc42 |
| H43 | fa1 | fb8 | fc43 |
| H44 | fa1 | fb8 | fc44 |
| H45 | fa1 | fb8 | fc45 |
| H46 | fa1 | fb8 | fc46 |
| H47 | fa1 | fb8 | fc47 |
| H48 | fa1 | fb8 | fc48 |
| H49 | fa1 | fb8 | fc49 |
| H50 | fa1 | fb8 | fc50 |
| H51 | fa1 | fb8 | fc51 |
| H52 | fa1 | fb8 | fc52 |
| I1 | fa1 | fb9 | fc2 |
| I2 | fa1 | fb9 | fc3 |
| I3 | fa1 | fb9 | fc4 |
| I4 | fa1 | fb9 | fc6 |
| I5 | fa1 | fb9 | fc7 |
| I6 | fa1 | fb9 | fc17 |
| I7 | fa1 | fb9 | fc18 |
| I8 | fa1 | fb9 | fc19 |
| I9 | fa1 | fb9 | fc20 |
| I10 | fa1 | fb9 | fc21 |
| I11 | fa1 | fb9 | fc22 |
| I12 | fa1 | fb9 | fc23 |
| I13 | fa1 | fb9 | fc24 |
| I14 | fa1 | fb9 | fc25 |
| I15 | fa1 | fb9 | fc26 |
| I16 | fa1 | fb9 | fc27 |
| I17 | fa1 | fb9 | fc28 |
| I18 | fa1 | fb9 | fc29 |
| I19 | fa1 | fb9 | fc30 |
| I20 | fa1 | fb9 | fc31 |
| I21 | fa1 | fb9 | fc32 |
| I22 | fa1 | fb9 | fc33 |
| I23 | fa1 | fb9 | fc34 |
| I24 | fa1 | fb9 | fc35 |
| I25 | fa1 | fb9 | fc36 |
| I26 | fa1 | fb9 | fc37 |
| I27 | fa1 | fb9 | fc38 |
| I28 | fa1 | fb9 | fc39 |
| I29 | fa1 | fb9 | fc40 |
| I30 | fa1 | fb9 | fc41 |
| I31 | fa1 | fb9 | fc42 |
| I32 | fa1 | fb9 | fc43 |
| I33 | fa1 | fb9 | fc44 |
| I34 | fa1 | fb9 | fc45 |
| I35 | fa1 | fb9 | fc46 |
| I36 | fa1 | fb9 | fc47 |
| I37 | fa1 | fb9 | fc48 |
| I38 | fa1 | fb9 | fc49 |
| I39 | fa1 | fb9 | fc50 |
| I40 | fa1 | fb9 | fc51 |
| I41 | fa1 | fb9 | fc52 |
| I42 | fa1 | fb9 | fc53 |
| J1 | fa1 | fb10 | fc1 |
| J2 | fa1 | fb10 | fc2 |
| J3 | fa1 | fb10 | fc3 |
| J4 | fa1 | fb10 | fc4 |
| J5 | fa1 | fb10 | fc5 |
| J6 | fa1 | fb10 | fc6 |
| J7 | fa1 | fb10 | fc7 |
| J8 | fa1 | fb10 | fc8 |
| J9 | fa1 | fb10 | fc9 |
| J10 | fa1 | fb10 | fc10 |
| J11 | fa1 | fb10 | fc11 |
| J12 | fa1 | fb10 | fc12 |
| J13 | fa1 | fb10 | fc13 |
| J14 | fa1 | fb10 | fc14 |
| J15 | fa1 | fb10 | fc15 |
| J16 | fa1 | fb10 | fc16 |
| J17 | fa1 | fb10 | fc17 |
| J18 | fa1 | fb10 | fc18 |
| J19 | fa1 | fb10 | fc19 |
| J20 | fa1 | fb10 | fc20 |
| J21 | fa1 | fb10 | fc21 |
| J22 | fa1 | fb10 | fc22 |
| J23 | fa1 | fb10 | fc23 |
| J24 | fa1 | fb10 | fc24 |
| J25 | fa1 | fb10 | fc25 |
| J26 | fa1 | fb10 | fc26 |
| J27 | fa1 | fb10 | fc27 |
| J28 | fa1 | fb10 | fc28 |
| J29 | fa1 | fb10 | fc29 |
| J30 | fa1 | fb10 | fc30 |
| J31 | fa1 | fb10 | fc31 |
| J32 | fa1 | fb10 | fc32 |
| J33 | fa1 | fb10 | fc33 |
| J34 | fa1 | fb10 | fc34 |
| J35 | fa1 | fb10 | fc35 |
| J36 | fa1 | fb10 | fc36 |
| J37 | fa1 | fb10 | fc37 |
| J38 | fa1 | fb10 | fc38 |
| J39 | fa1 | fb10 | fc39 |
| J40 | fa1 | fb10 | fc40 |
| J41 | fa1 | fb10 | fc41 |
| J42 | fa1 | fb10 | fc42 |
| J43 | fa1 | fb10 | fc43 |
| J44 | fa1 | fb10 | fc44 |
| J45 | fa1 | fb10 | fc45 |
| J46 | fa1 | fb10 | fc46 |
| J47 | fa1 | fb10 | fc47 |
| J48 | fa1 | fb10 | fc48 |
| J49 | fa1 | fb10 | fc49 |
| J50 | fa1 | fb10 | fc50 |
| J51 | fa1 | fb10 | fc51 |
| J52 | fa1 | fb10 | fc52 |
| K1 | fa1 | fb11 | fc2 |
| K2 | fa1 | fb11 | fc3 |
| K3 | fa1 | fb11 | fc4 |
| K4 | fa1 | fb11 | fc5 |
| K5 | fa1 | fb11 | fc6 |
| K6 | fa1 | fb11 | fc7 |
| K7 | fa1 | fb11 | fc17 |
| K8 | fa1 | fb11 | fc18 |
| K9 | fa1 | fb11 | fc19 |
| K10 | fa1 | fb11 | fc20 |
| K11 | fa1 | fb11 | fc21 |
| K12 | fa1 | fb11 | fc22 |
| K13 | fa1 | fb11 | fc23 |
| K14 | fa1 | fb11 | fc24 |
| K15 | fa1 | fb11 | fc25 |
| K16 | fa1 | fb11 | fc26 |
| K17 | fa1 | fb11 | fc27 |
| K18 | fa1 | fb11 | fc28 |
| K19 | fa1 | fb11 | fc29 |
| K20 | fa1 | fb11 | fc30 |
| K21 | fa1 | fb11 | fc31 |
| K22 | fa1 | fb11 | fc32 |
| K23 | fa1 | fb11 | fc33 |
| K24 | fa1 | fb11 | fc34 |
| K25 | fa1 | fb11 | fc35 |
| K26 | fa1 | fb11 | fc36 |
| K27 | fa1 | fb11 | fc37 |
| K28 | fa1 | fb11 | fc38 |
| K29 | fa1 | fb11 | fc39 |
| K30 | fa1 | fb11 | fc40 |
| K31 | fa1 | fb11 | fc41 |
| K32 | fa1 | fb11 | fc42 |
| K33 | fa1 | fb11 | fc43 |
| K34 | fa1 | fb11 | fc44 |
| K35 | fa1 | fb11 | fc45 |
| K36 | fa1 | fb11 | fc46 |
| K37 | fa1 | fb11 | fc47 |
| K38 | fa1 | fb11 | fc48 |
| K39 | fa1 | fb11 | fc49 |
| K40 | fa1 | fb11 | fc50 |
| K41 | fa1 | fb11 | fc51 |
| K42 | fa1 | fb11 | fc52 |

The light emitting element ED of one or more embodiments may include at least one selected from among the compounds listed in Compound Combination Table 1 in a hole transport region HTR. The amine compound according to one or more embodiments includes a first substituent, a second substituent and a third substituent, which are directly or indirectly connected to the core nitrogen atom, (e.g., with the nitrogen atom of an amine), and may accomplish (e.g., provide or contribute to) the long lifetime of a light emitting element.

For example, the amine compound of one or more embodiments may include (e.g., may essentially include) a first substituent which is directly connected to the core nitrogen atom, (e.g., with the nitrogen atom of the amine), and a second substituent. The amine compound of one or more embodiments includes a benzonaphthothiophene group directly connected to the core nitrogen atom, (e.g., with the nitrogen atom of the amine) at carbon position 10, as the first substituent, and includes a substituted or unsubstituted dibenzofuran group, a substituted or unsubstituted dibenzothiophene group, or a substituted or unsubstituted fluorenyl group, as the second substituent. The amine compound of one or more embodiments may have improved orientation through the intermolecular interaction in the first substituent and the second substituent, and may show excellent or suitable electrical stability and high charge transport capacity. Accordingly, when the amine compound of one or more embodiments is applied in a light emitting element, element lifetime may be improved.

In the light emitting element ED of one or more embodiments, the hole transport region HTR may further include a compound represented by Formula H-1.

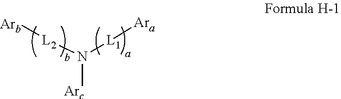

In Formula H-1, L₁ and L₂ may each independently be a direct linkage, a substituted or unsubstituted arylene group of 6 to 30 ring-forming carbon atoms, or a substituted or unsubstituted heteroarylene group of 2 to 30 ring-forming carbon atoms. In Formula H-1, “a” and “b” may each independently be an integer of 0 to 10. In some embodiments, when “a” or “b” is an integer of 2 or more, multiple L₁ and L₂ may each independently be a substituted or unsubstituted arylene group of 6 to 30 ring-forming carbon atoms, or a substituted or unsubstituted heteroarylene group of 2 to 30 ring-forming carbon atoms.

In Formula H-1, Arₐ and Ar_{b} may each independently be a substituted or unsubstituted aryl group of 6 to 30 ring-forming carbon atoms, or a substituted or unsubstituted heteroaryl group of 2 to 30 ring-forming carbon atoms. In some embodiments, in Formula H-1, Ar_{c} may be a substituted or unsubstituted aryl group of 6 to 30 ring-forming carbon atoms.

The compound represented by Formula H-1 may be a monoamine compound. In some embodiments, the compound represented by Formula H-1 may be a diamine compound in which at least one among Arₐ to Ar_{c} includes an amine group as a substituent. In some embodiments, the compound represented by Formula H-1 may be a carbazole-based compound in which at least one among Arₐ and Ar_{b} includes a substituted or unsubstituted carbazole group, or a fluorene-based compound in which at least one among Arₐ and Ar_{b} includes a substituted or unsubstituted fluorene group.

The compound represented by Formula H-1 may be represented by any one selected from among the compounds in Compound Group H. However, the compounds listed in Compound Group H are only illustrations, and the compound represented by Formula H-1 is not limited to the compounds represented in Compound Group H.

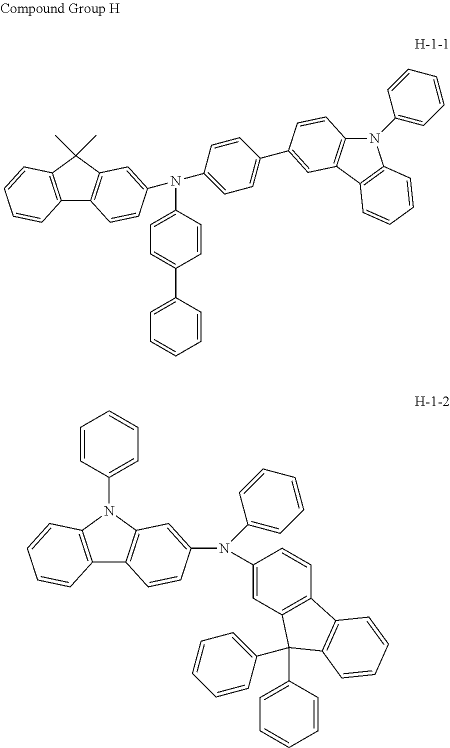 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1]

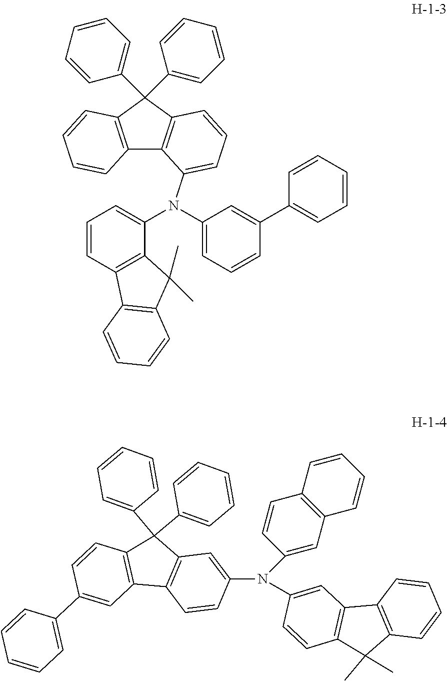 [SMILES: CC1(C)c2ccccc2-c2cccc(N(c3cccc(-c4ccccc4)c3)c3cccc4c3-c3ccccc3C4(c3ccccc3)c3ccccc3)c21 ; CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21]

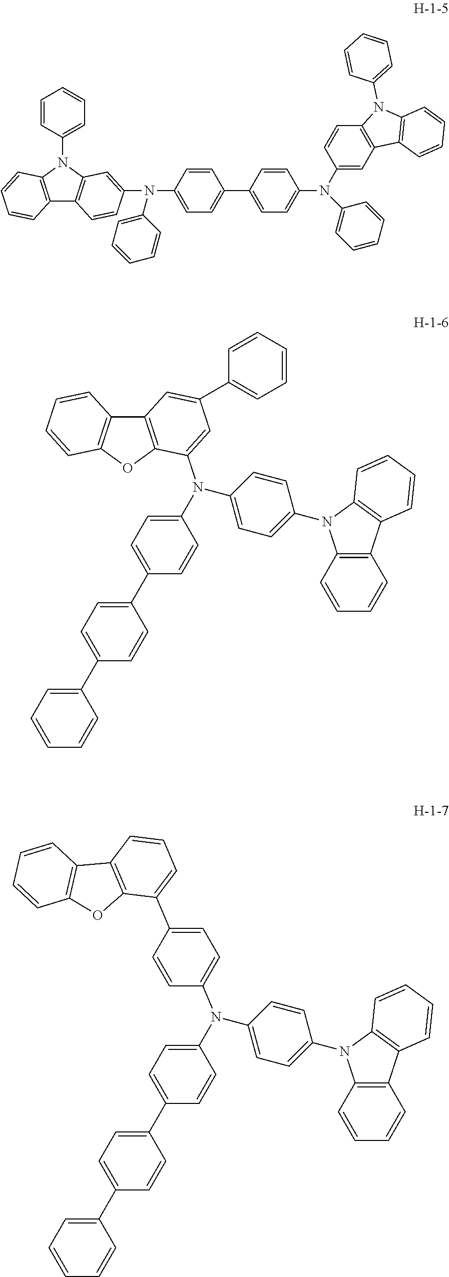 [SMILES: c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

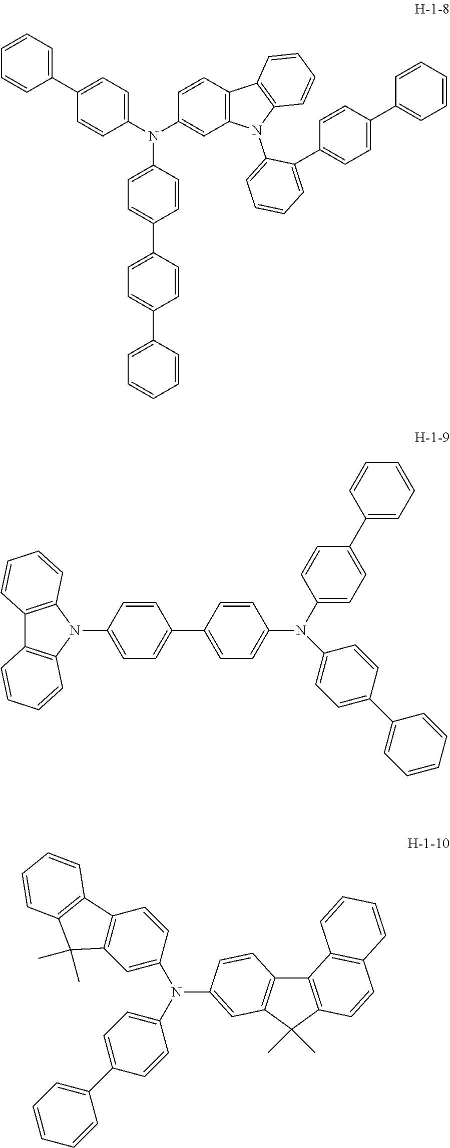 [SMILES: c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2ccccc2-c2ccc(N(c3ccc(-c4ccccc4)cc3)c3ccc4c(c3)C(C)(C)c3ccc5ccccc5c3-4)cc21]

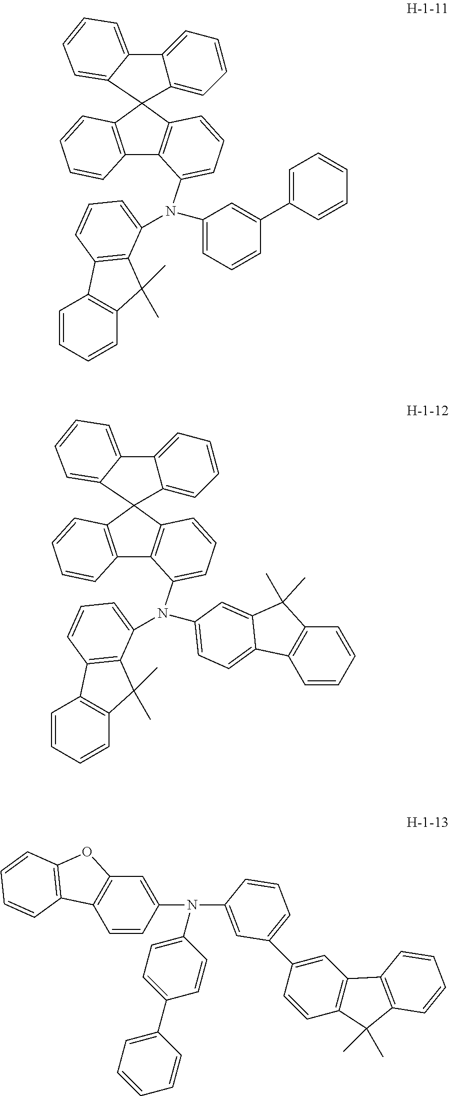 [SMILES: CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21]

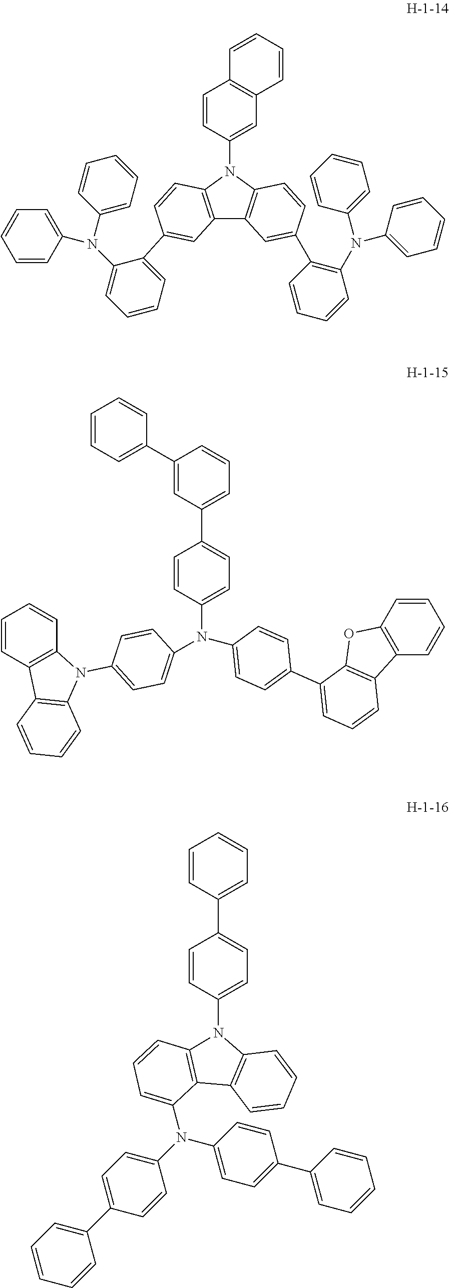 [SMILES: c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1]

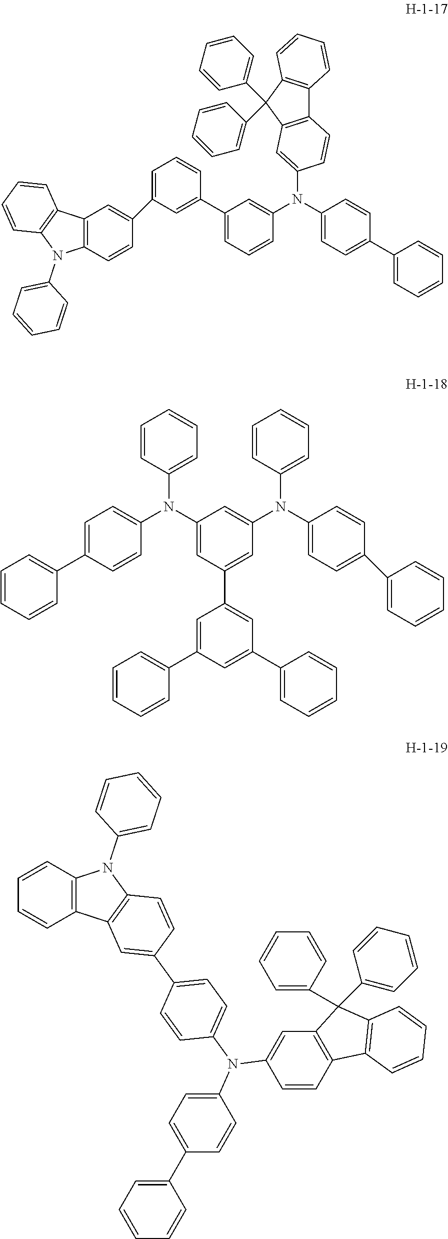 [SMILES: c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

In some embodiments, the hole transport region HTR may further include a suitable hole transport material.

For example, the hole transport region HTR may include a phthalocyanine compound such as copper phthalocyanine, N¹,N^{1′}-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino] triphenylamine (m-MTDATA), 4,4′,4″-tris(N,N-diphenylamino)triphenylamine (TDATA), 4,4′,4″-tris[N-(1-naphthyl)-N-phenylamino]-triphenylamine (1-TNATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(1-naphthalen-1-yl)-N, N′-diphenyl-benzidine (NPB or NPD), triphenylamine-containing polyetherketone (TPAPEK), 4-isopropyl-4′-methyldiphenyliodonium [tetrakis(pentafluorophenyl)borate], and/or dipyrazino[2,3-f:2′,3′-h] quinoxaline-2,3,6,7,10,11-hexacarbonitrile (HATCN).

The hole transport region HTR may include carbazole derivatives such as N-phenyl carbazole and polyvinyl carbazole, fluorene-based derivatives, triphenylamine-based derivatives such as 4,4′,4″-tris(N-carbazolyl)triphenylamine (TCTA), N,N′-bis(3-methylphenyl)-N, N′-diphenyl-[1,1′-biphenyl]-4,4′-diamine (TPD), N, N′-di(naphthalen-1-yl)-N,N′-diphenyl-benzidine (NPB), 4,4′-cyclohexylidene bis[N,N-bis(4-methylphenyl)benzeneamine] (TAPC), 4,4′-bis[N, N′-(3-tolyl)amino]-3,3′-dimethylbiphenyl (HMTPD), benzene1,3-bis(carbazol-9-yl)benzene(mCP), and/or the like.

In some embodiments, the hole transport region HTR may include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), and/or the like.

The hole transport region HTR may include the compounds of the hole transport region HTR in at least one among a hole injection layer HIL, a hole transport layer HTL, and/or an electron blocking layer EBL.

The thickness of the hole transport region HTR may be from about 100 Å to about 10,000 Å, for example, from about 100 Å to about 5,000 Å. When the hole transport region HTR includes a hole injection layer HIL, the thickness of the hole injection layer HIL may be, for example, from about 30 Å to about 1,000 Å. When the hole transport region HTR includes a hole transport layer HTL, the thickness of the hole transport layer HTL may be from about 30 Å to about 1,000 Å. For example, when the hole transport region HTR includes an electron blocking layer EBL, the thickness of the electron blocking layer EBL may be from about 10 Å to about 1,000 Å. When the thicknesses of the hole transport region HTR, the hole injection layer HIL, the hole transport layer HTL and the electron blocking layer EBL satisfy the above-described ranges, satisfactory hole transport properties may be achieved without substantial increase of a driving voltage.

The hole transport region HTR may further include a charge generating material that is configured to enhance or to increase conductivity in addition to the described materials. The charge generating material may be dispersed uniformly or non-uniformly in the hole transport region HTR. The charge generating material may be, for example, a p-dopant. The p-dopant may include at least one of metal halide compounds, quinone derivatives, metal oxides, and cyano group-containing compounds, without limitation. For example, the p-dopant may include metal halide compounds such as CuI and RbI, quinone derivatives such as tetracyanoquinodimethane (TCNQ) and 2,3,5,6-tetrafluoro-7,7′,8,8-tetracyanoquinodimethane (F4-TCNQ), metal oxides such as tungsten oxide and molybdenum oxide, cyano group-containing compounds such as dipyrazino[2,3-f: 2′,3′-h] quinoxaline-2,3,6,7,10,11-hexacarbonitrile (HATCN) and 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), and/or the like, but is not limited thereto.

As described elsewhere herein, the hole transport region HTR may further include a buffer layer in addition to the hole injection layer HIL, the hole transport layer HTL and the electron blocking layer EBL. The buffer layer may compensate resonance distance according to the wavelength of light emitted from an emission layer EML and may enhance or increase emission efficiency. The materials included in the buffer layer may utilize materials which may be included in the hole transport region HTR.

The emission layer EML is provided on the hole transport region HTR. The emission layer EML may have a thickness of, for example, about 100 Å to about 1,000 Å or about 100 Å to about 300 Å. The emission layer EML may have a single layer formed utilizing a single material, a single layer formed utilizing multiple different materials, or a multilayer structure having multiple layers formed utilizing multiple different materials.

In the light emitting element ED of one or more embodiments, the emission layer EML may be to emit blue light. The light emitting element ED of one or more embodiments may include the amine compound of one or more embodiments in a hole transport region HTR and may show high efficiency emission and long-life (e.g., long lifetime) characteristics in a blue emission region. However, one or more embodiments of the present disclosure is not limited thereto.

In the light emitting element ED of one or more embodiments, the emission layer EML may include anthracene derivatives, pyrene derivatives, fluoranthene derivatives, chrysene derivatives, dihydrobenzanthracene derivatives, and/or triphenylene derivatives. For example, the emission layer EML may include anthracene derivatives or pyrene derivatives.

In the light emitting elements ED of embodiments, shown in FIG. 3 to FIG. 6, the emission layer EML may include a host and a dopant, and the emission layer EML may include a compound represented by Formula E-1. The compound represented by Formula E-1 may be utilized as a fluorescence host material.

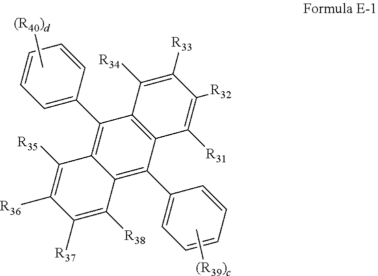

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form a ring. In some embodiments, R₃₁ to R₄₀ may be combined with an adjacent group to form a saturated hydrocarbon ring, an unsaturated hydrocarbon ring, a saturated heterocycle, or an unsaturated heterocycle.

In Formula E-1, “c” and “d” may each independently be an integer of 0 to 5.

Formula E-1 may be represented by any one selected from among

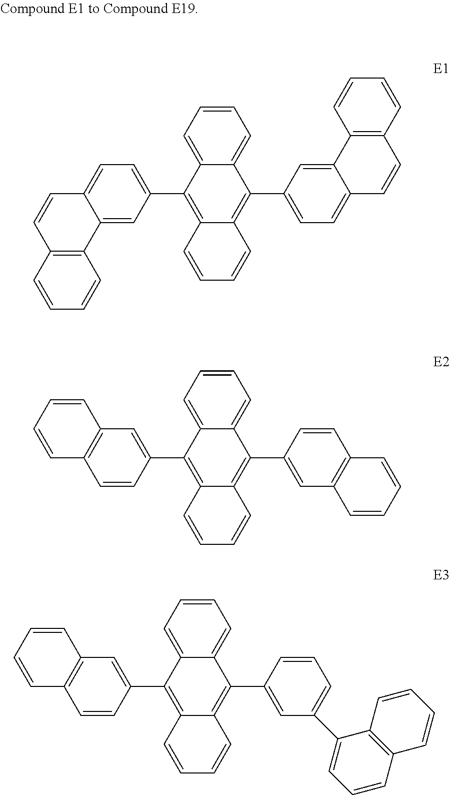

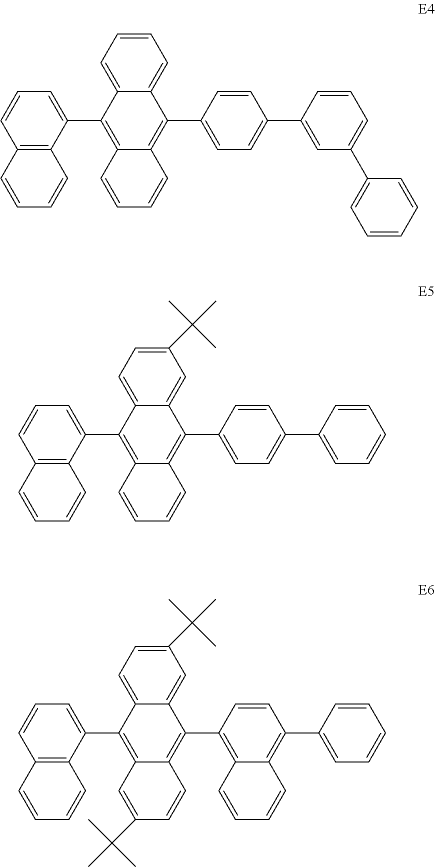

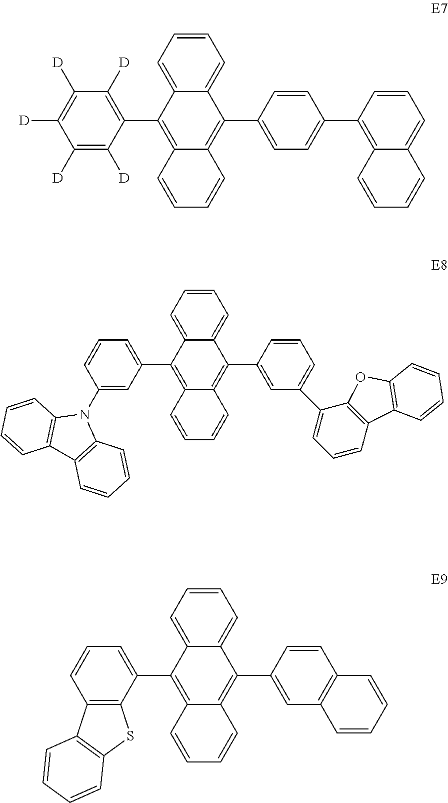 [SMILES: [2H]c1c([2H])c([2H])c(-c2c3ccccc3c(-c3ccc(-c4cccc5ccccc45)cc3)c3ccccc23)c([2H])c1[2H] ; c1cc(-c2c3ccccc3c(-c3cccc(-n4c5ccccc5c5ccccc54)c3)c3ccccc23)cc(-c2cccc3c2oc2ccccc23)c1 ; c1ccc2cc(-c3c4ccccc4c(-c4cccc5c4sc4ccccc45)c4ccccc34)ccc2c1]

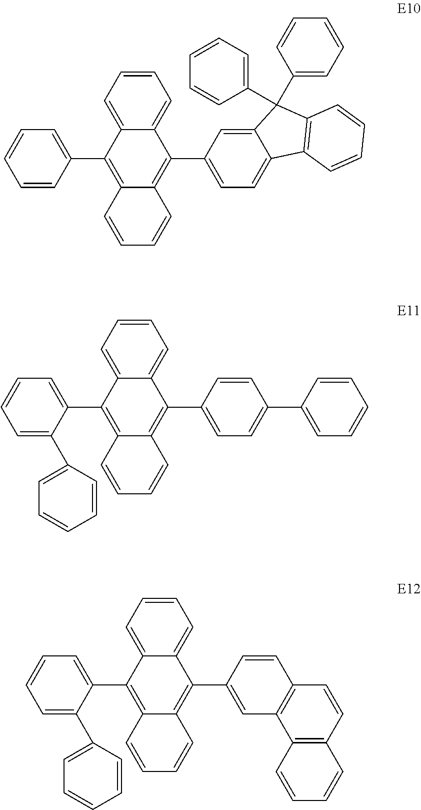

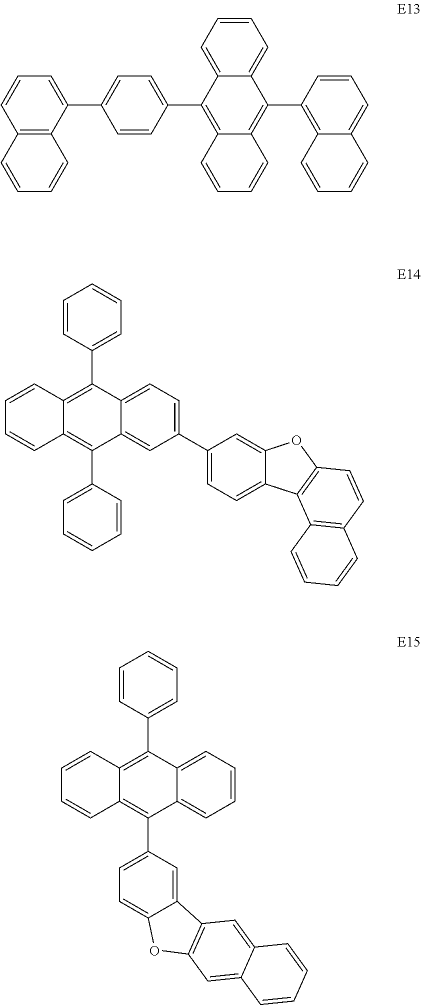

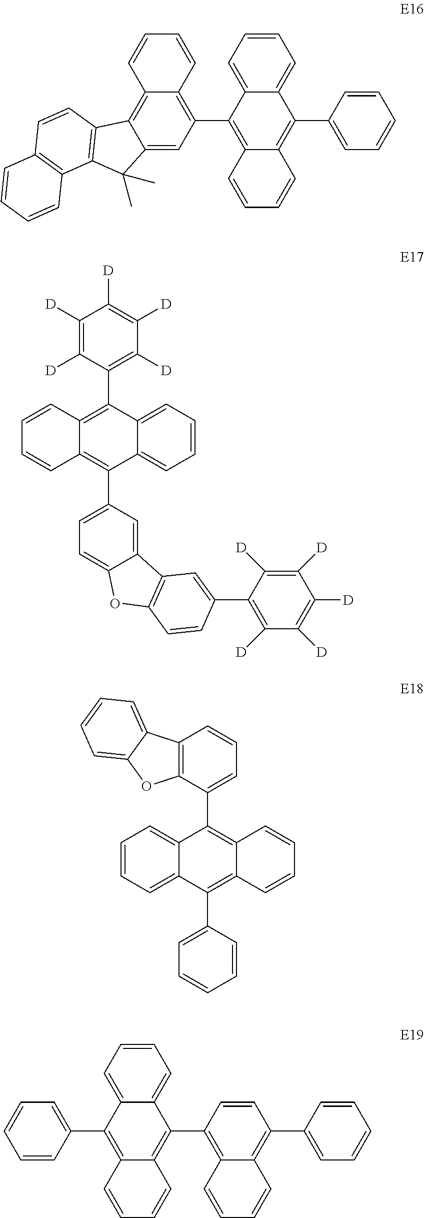

In one or more embodiments, the emission layer EML may include a compound represented by Formula E-2a or Formula E-2b. The compound represented by Formula E-2a or Formula E-2b may be utilized as a phosphorescence host material.

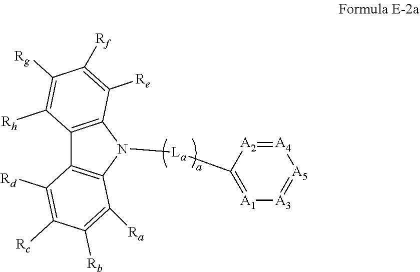

In Formula E-2a, “a” may be an integer of 0 to 10, La may be a direct linkage, a substituted or unsubstituted arylene group of 6 to 30 ring-forming carbon atoms, or a substituted or unsubstituted heteroarylene group of 2 to 30 ring-forming carbon atoms. In some embodiments, when “a” is an integer of 2 or more, multiple La may each independently be a substituted or unsubstituted arylene group of 6 to 30 ring-forming carbon atoms, or a substituted or unsubstituted heteroarylene group of 2 to 30 ring-forming carbon atoms.

In some embodiments, in Formula E-2a, A₁ to A₅ may each independently be N or CRᵢ. Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. For example, Rₐ to Rᵢ may be combined with an adjacent group to form a hydrocarbon ring or a heterocycle including N, O, S, etc. as a ring-forming atom.

In some embodiments, in Formula E-2a, two or three selected from A1 to A₅ may be N, and the remainder may be CRᵢ.

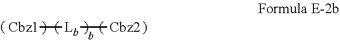

1 In Formula E-2b, Cbz1 and Cbz2 may each independently be an unsubstituted carbazole group, or a carbazole group substituted with an aryl group of 6 to 30 ring-forming carbon atoms. L_{b} may be a direct linkage, a substituted or unsubstituted arylene group of 6 to 30 ring-forming carbon atoms, or a substituted or unsubstituted heteroarylene group of 2 to 30 ring-forming carbon atoms. In Formula E-2b, “b” is an integer of 0 to 10, and when “b” is an integer of 2 or more, multiple L_{b} may each independently be a substituted or unsubstituted arylene group of 6 to 30 ring-forming carbon atoms, or a substituted or unsubstituted heteroarylene group of 2 to 30 ring-forming carbon atoms.

The compound represented by Formula E-2a or Formula E-2b may be represented by any one selected from among the compounds in Compound Group E-2. However, the compounds listed in Compound Group E-2 are only illustrations, and the compound represented by Formula E-2a or Formula E-2b is not limited to the compounds represented in Compound Group E-2.

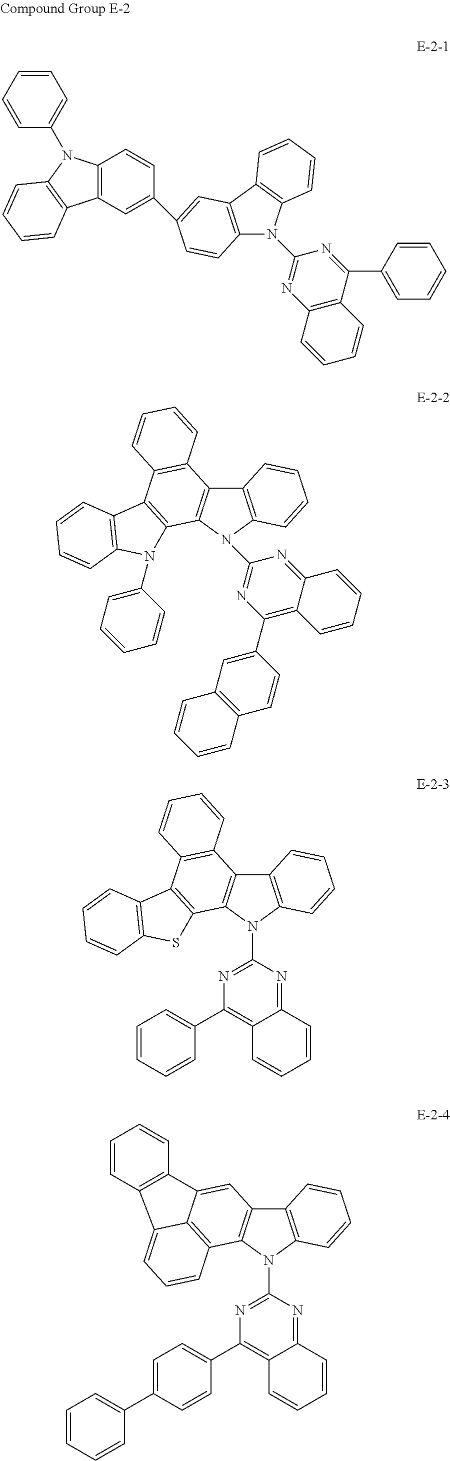

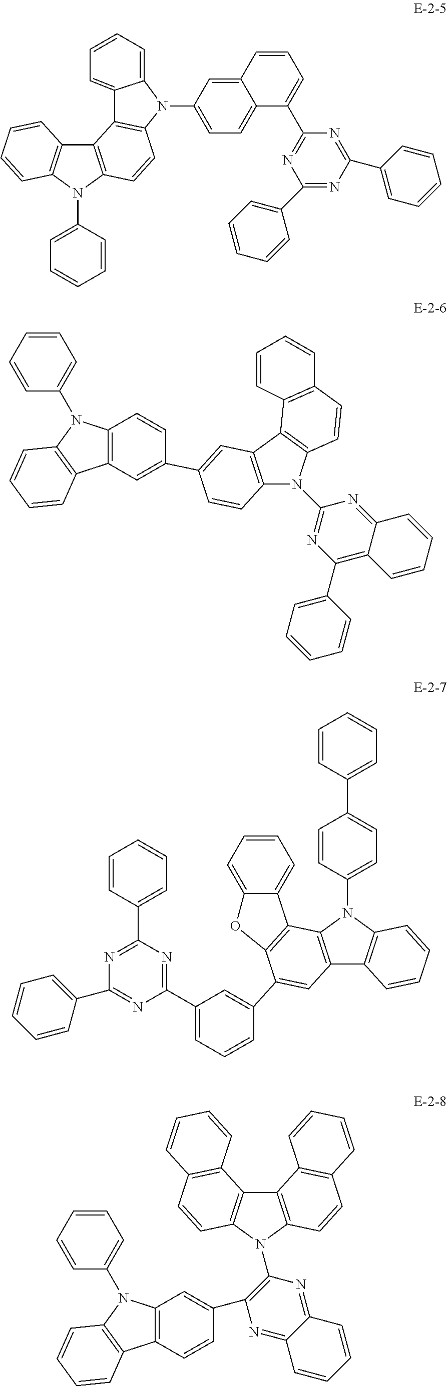 [SMILES: c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1]

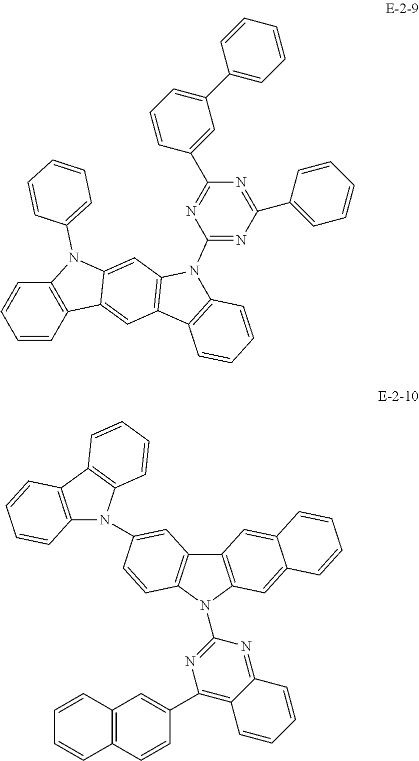 [SMILES: c1ccc(-c2cccc(-c3nc(-c4ccccc4)nc(-n4c5ccccc5c5cc6c7ccccc7n(-c7ccccc7)c6cc54)n3)c2)cc1 ; c1ccc2cc(-c3nc(-n4c5ccc(-n6c7ccccc7c7ccccc76)cc5c5cc6ccccc6cc54)nc4ccccc34)ccc2c1]

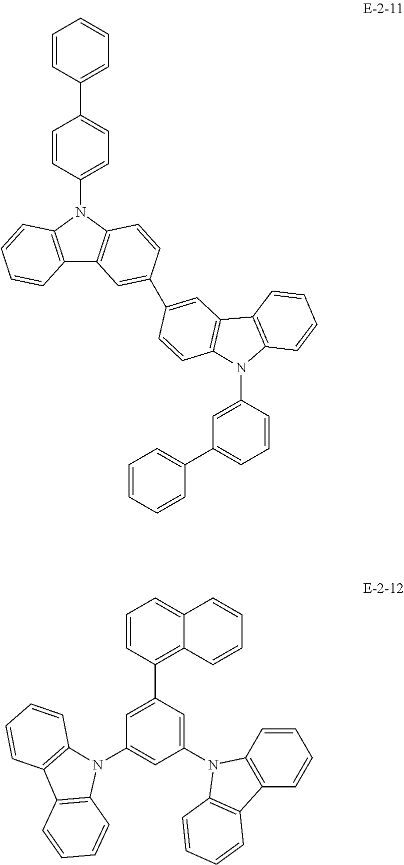 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc2c(-c3cc(-n4c5ccccc5c5ccccc54)cc(-n4c5ccccc5c5ccccc54)c3)cccc2c1]

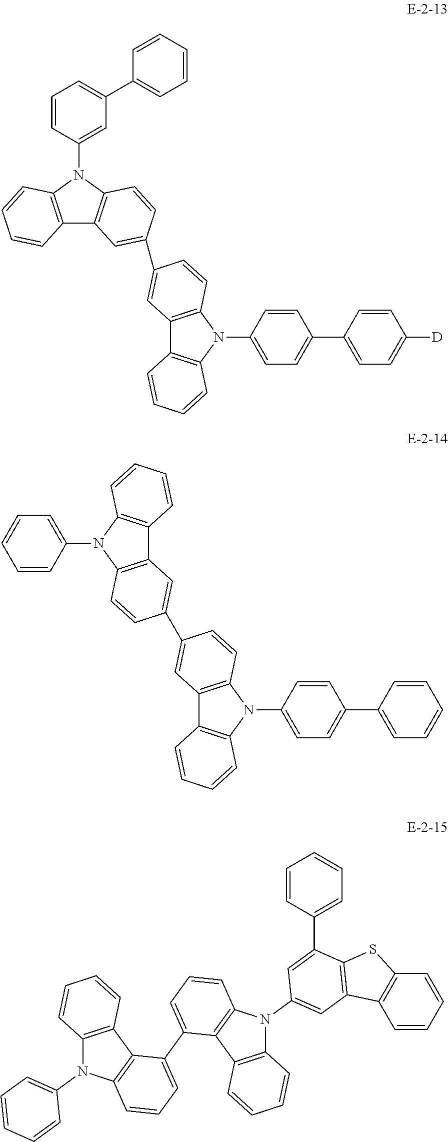 [SMILES: [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

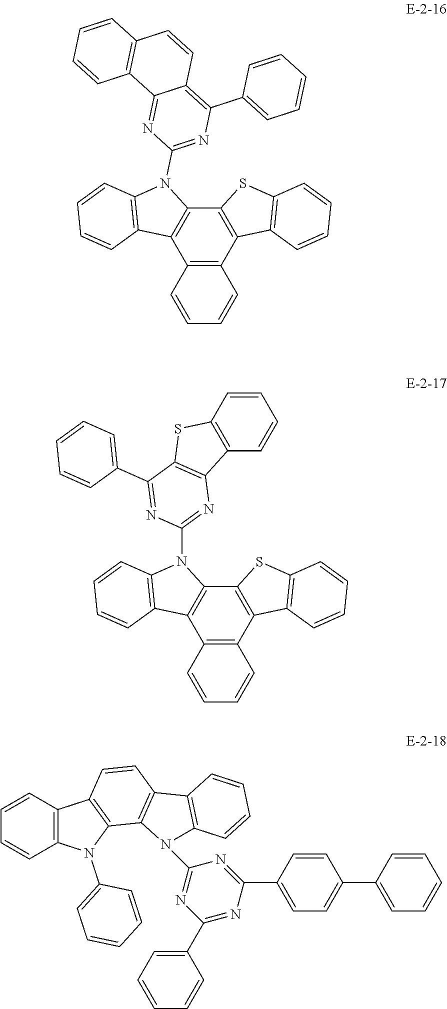 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1]

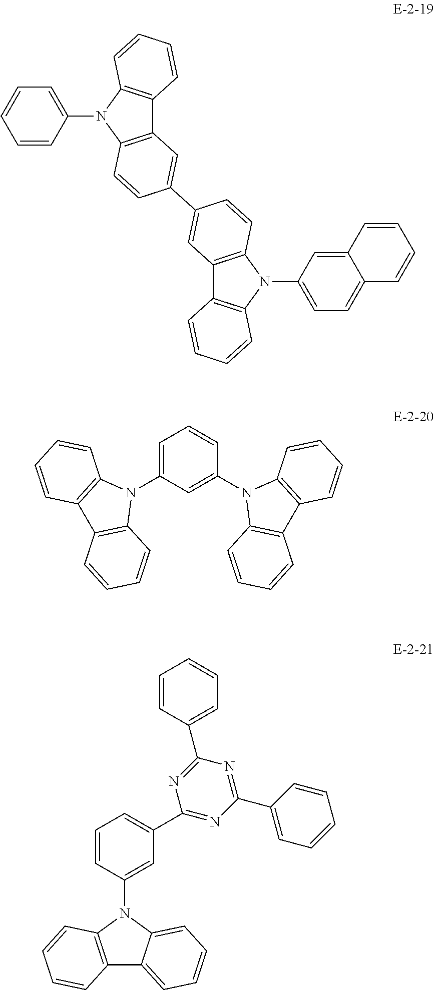 [SMILES: c1ccc(-n2c3ccccc3c3cc(-c4ccc5c(c4)c4ccccc4n5-c4ccc5ccccc5c4)ccc32)cc1 ; c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1]

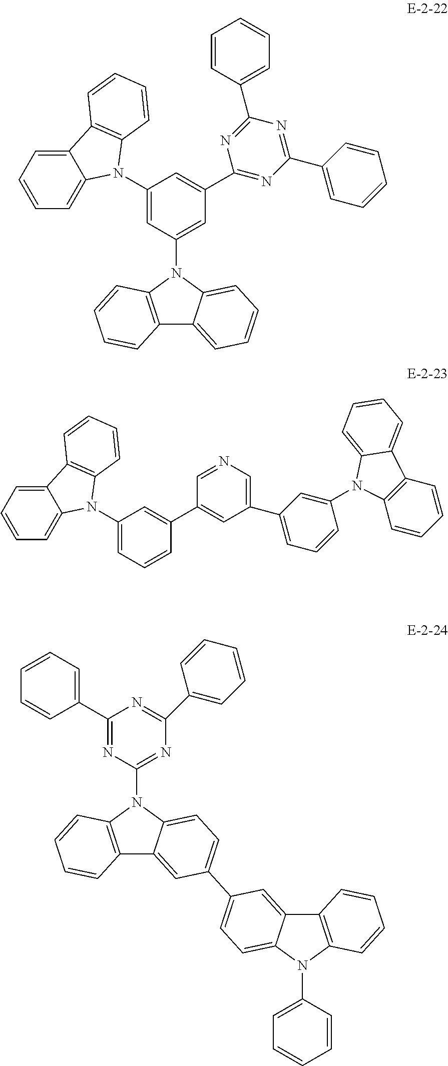 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a common material well-suitable in the art as a host material. For example, the emission layer EML may include as a host material, at least one of bis (4-(9H-carbazol-9-yl) phenyl) diphenylsilane (BCPDS), (4-(1-(4-(diphenylamino) phenyl) cyclohexyl) phenyl) diphenyl-phosphine oxide (POPCPA), bis[2-(diphenylphosphino)phenyl] ether oxide (DPEPO), 4,4′-bis(carbazol-9-yl)biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazole-2-yl)benzene (TPBi). However, one or more embodiments of the present disclosure is not limited thereto. For example, tris(8-hydroxyquinolinato)aluminum (Alq₃), 9,10-di(naphthalene-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), etc. may be utilized as the host material.

The emission layer EML may include a compound represented by Formula M-a or Formula M-b. The compound represented by Formula M-a or Formula M-b may be utilized as a phosphorescence dopant material. In some embodiments, the compound represented by Formula M-a or Formula M-b may be utilized as an auxiliary dopant material.

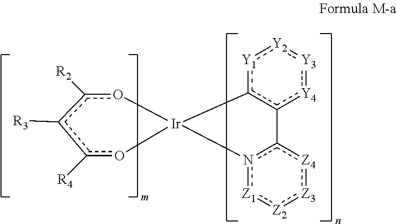

In Formula M-a, Y₁ to Y₄, and Z₁ to Z₄ may each independently be CR₁ or N, and R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. In Formula M-a, “m” is 0 or 1, and “n” is 2 or 3. In Formula M-a, when “m” is 0, then “n” is 3, and when “m” is 1, then “n” is 2.

In one or more embodiments, the compound represented by Formula M-a may be utilized as a phosphorescence dopant.

In one or more embodiments, the compound represented by Formula M-a may be represented by any one selected from among Compounds M-a1 to M-a25. However, Compounds M-a1 to M-a25 are illustrations, and the compound represented by Formula M-a is not limited to the compounds represented by Compounds M-a1 to M-a25.

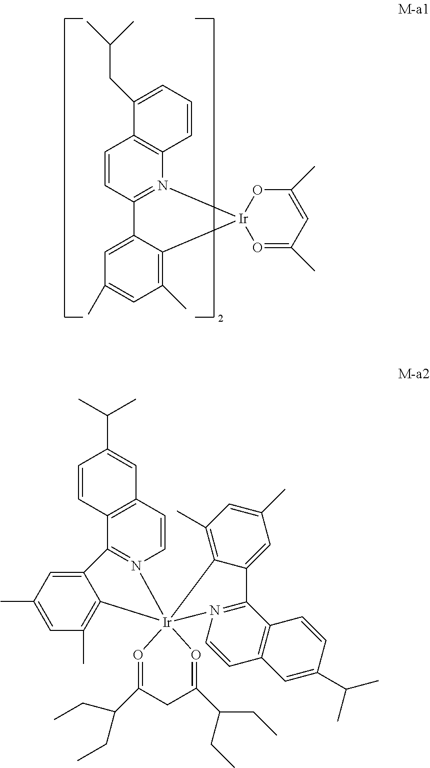

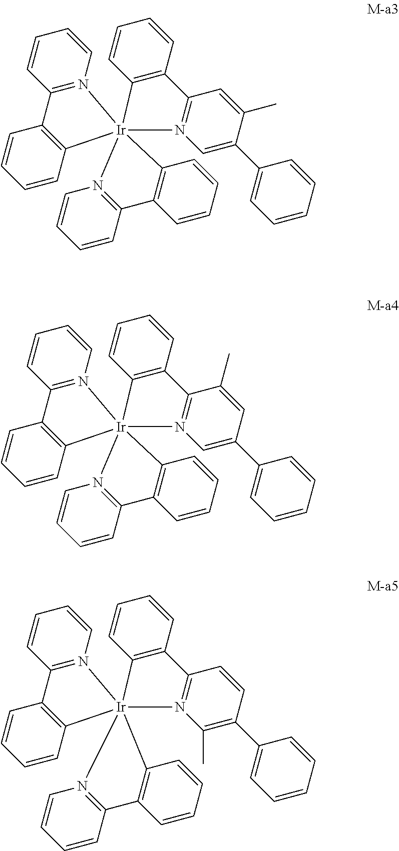

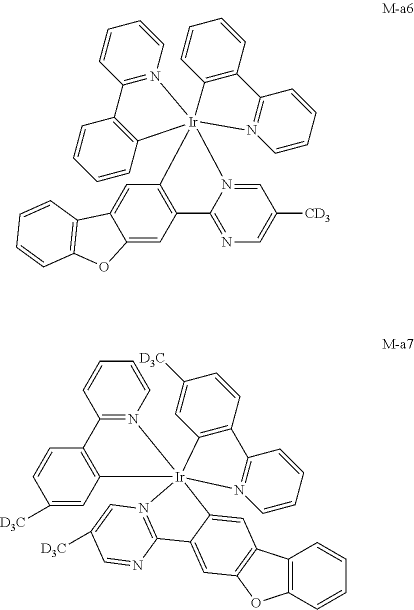

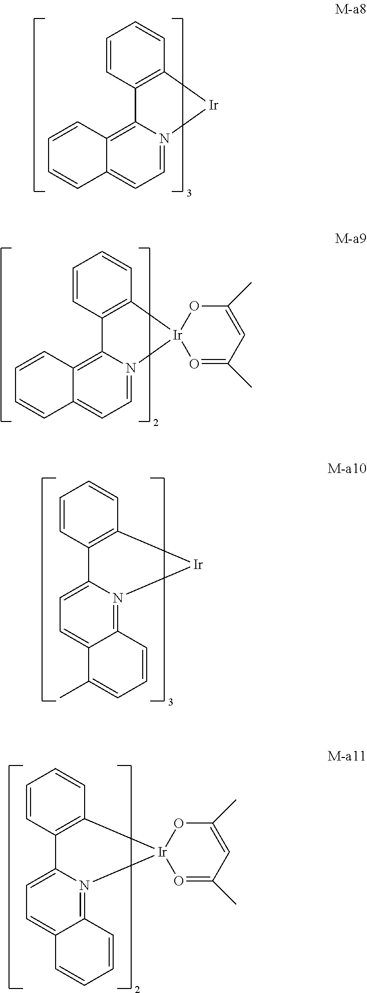

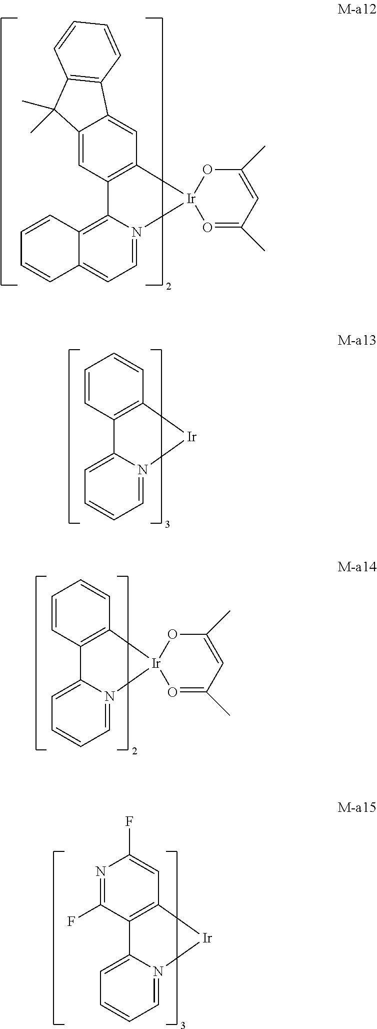

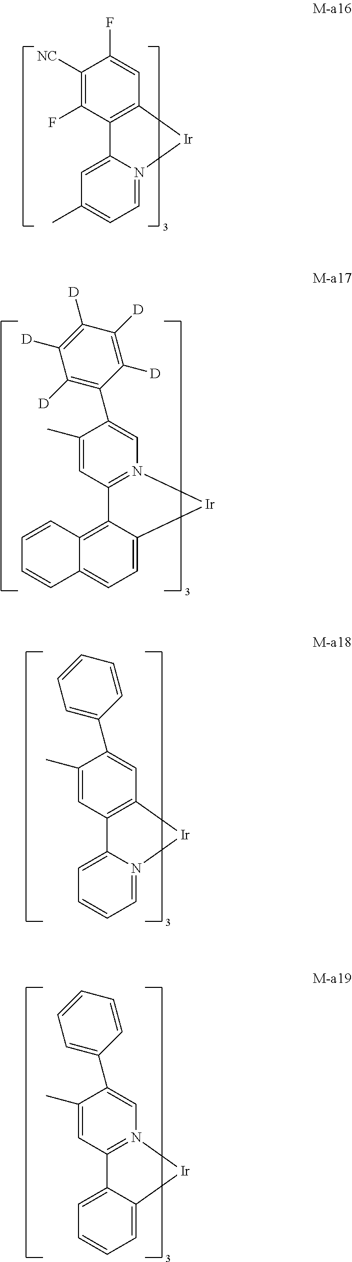

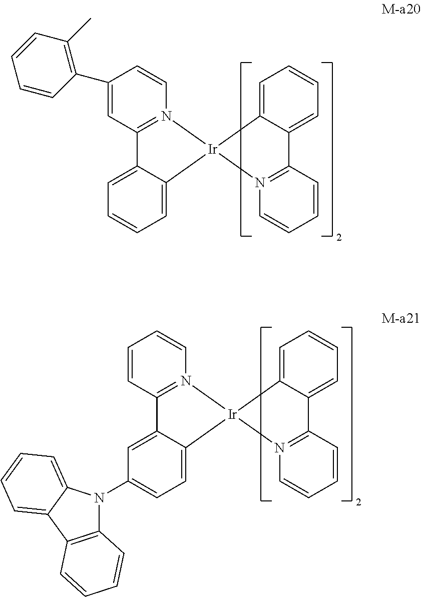

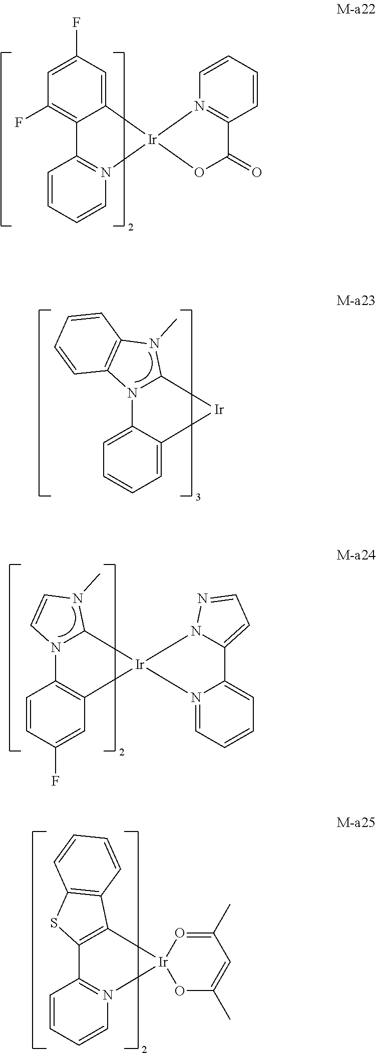

In an embodiment, Compound M-a1 and Compound M-a2 may be utilized as red dopant materials, and Compound M-a3 to Compound M-a7 may be utilized as green dopant materials.

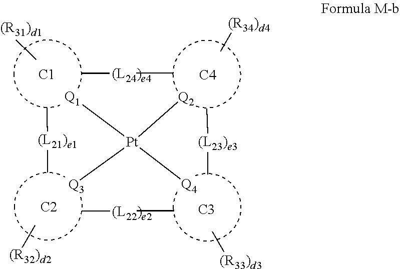

In Formula M-b, Q₁ to Q₄ may each independently be C or N, C1 to C4 may each independently be a substituted or unsubstituted hydrocarbon ring of 5 to 30 ring-forming carbon atoms, or a substituted or unsubstituted heterocycle of 2 to 30 ring-forming carbon atoms. L₂₁ to L₂₄ may each independently be a direct linkage,

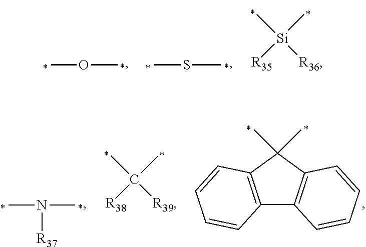

a substituted or unsubstituted divalent alkyl group of 1 to 20 carbon atoms, a substituted or unsubstituted arylene group of 6 to 30 ring-forming carbon atoms, or a substituted or unsubstituted heteroarylene group of 2 to 30 ring-forming carbon atoms, and e1 to e4 may each independently be 0 or 1. R₃₁ to R₃₉ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring, and d1 to d4 may each independently be an integer of 0 to 4.

The compound represented by Formula M-b may be utilized as a blue phosphorescence dopant or a green phosphorescence dopant. In some embodiments, the compound represented by Formula M-b may be an auxiliary dopant in one or more embodiments and may be further included in the emission layer EML.

The compound represented by Formula M-b may be represented by any one selected from among Compound M-b-1 to Compound M-b-11. However, the compounds are illustrations, and the compound represented by Formula M-b is not limited to Compound M-b-1 to Compound M-b-11.

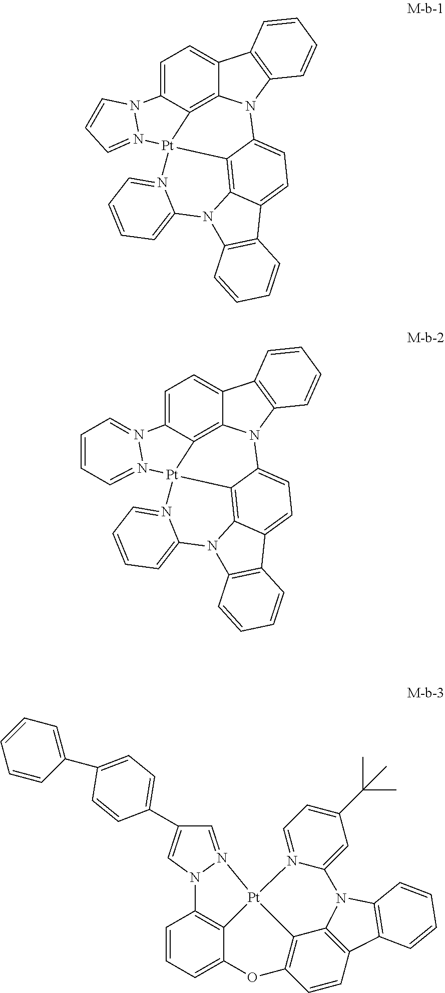

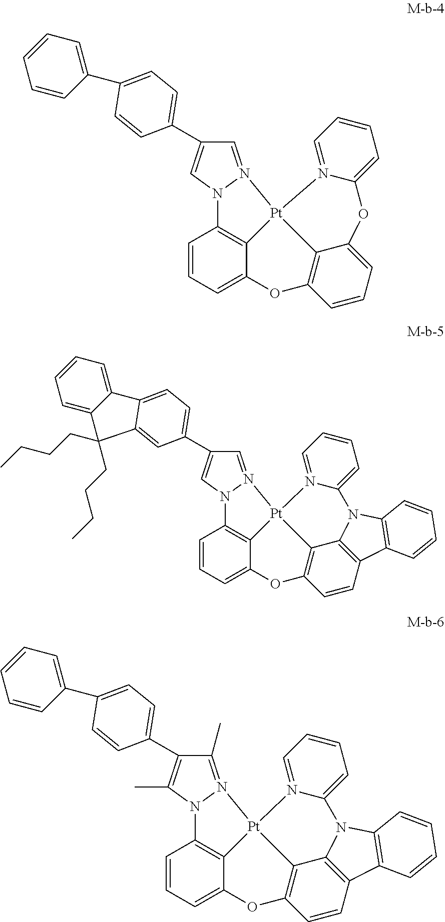

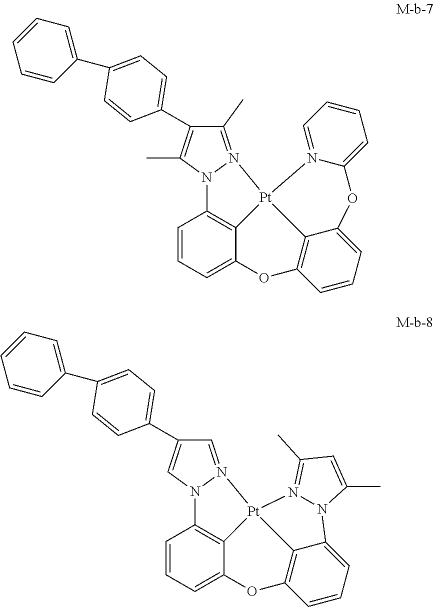

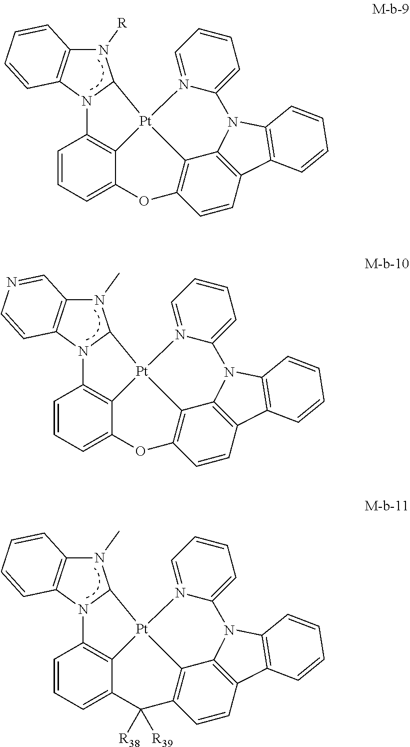

In the compounds M-b-9 and M-b-11, R, R₃₈, and R₃₉ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

The emission layer EML may include any one selected from among Formula F-a to Formula F-c. In an embodiment, the compounds represented by Formula F-a to Formula F-c may be utilized as fluorescence dopant materials.

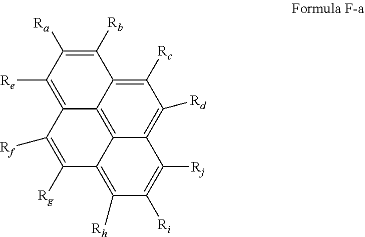

In Formula F-a, two selected from Rₐ to Rⱼ may each independently be substituted with *-NA₁Ar₂. The remainder not substituted with *-NA₁Ar₂ among Rₐ to Rⱼ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In *-NA₁Ar₂, Ar₁ and Ar₂ may each independently be a substituted or unsubstituted aryl group of 6 to 30 ring-forming carbon atoms, or a substituted or unsubstituted heteroaryl group of 2 to 30 ring-forming carbon atoms. For example, at least one among Ar₁ and Ar₂ may be a heteroaryl group including O or S as a ring-forming atom.

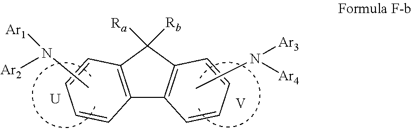

In Formula F-b, Rₐ and R_{b} may each independently be a hydrogen atom, a deuterium atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form a ring.

In Formula F-b, Ar₁ to Ar₄ may each independently be a substituted or unsubstituted aryl group of 6 to 30 ring-forming carbon atoms, or a substituted or unsubstituted heteroaryl group of 2 to 30 ring-forming carbon atoms.

In Formula F-b, U and V may each independently be a substituted or unsubstituted hydrocarbon ring of 5 to 30 ring-forming carbon atoms, or a substituted or unsubstituted heterocycle of 2 to 30 ring-forming carbon atoms.

In Formula F-b, the number of rings represented by U and V may each independently be 0 or 1. For example, in Formula F-b, when the number of U or Vis 1, one ring forms a fused ring at the designated part by U or V, and when the number of U or V is 0, a ring is not present at the designated part by U or V. For example, when the number of U is 0, and the number of V is 1, or when the number of U is 1, and the number of V is 0, a fused ring having the fluorene core of Formula F-b may be a ring compound with four rings. In some embodiments, when the number of both (e.g., simultaneously) U and V is 0, the fused ring of Formula F-b may be a ring compound with three rings. In some embodiments, when the number of both (e.g., simultaneously) U and V is 1, a fused ring having the fluorene core of Formula F-b may be a ring compound with five rings.

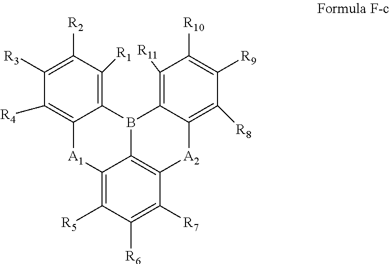

In Formula F-c, A₁ and A₂ may each independently be O, S, Se, or NRₘ, and Rₘ may be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. R₁ to R₁₁ may each independently be a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring.

In Formula F-c, A₁ and A₂ may each independently be combined with the substituents of an adjacent ring to form a fused ring. For example, when A₁ and A₂ may each independently be NRₘ, A₁ may be combined with R₄ or R₅ to form a ring. In some embodiments, A₂ may be combined with R₇ or R₈ to form a ring.

In one or more embodiments, the emission layer EML may include as a suitable dopant material, styryl derivatives (for example, 1,4-bis[2-(3-N-ethylcarbazoryl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (N-BDAVBi), and 4,4′-bis[2-(4-(N,N-diphenylamino)phenyl)vinyl]biphenyl (DPAVBi)), perylene and the derivatives thereof (for example, 2,5,8,11-tetra-t-butylperylene (TBP)), pyrene and the derivatives thereof (for example, 1,1′-dipyrene, 1,4-dipyrenylbenzene, and 1,4-bis(N,N-diphenylamino)pyrene), and/or the like.

In one or more embodiments, when multiple emission layers EML are included, at least one emission layer EML may include a suitable phosphorescence dopant material. For example, the phosphorescence dopant may utilize a metal complex including iridium (Ir), platinum (Pt), osmium (Os), gold (Au), titanium (Ti), zirconium (Zr), hafnium (Hf), europium (Eu), terbium (Tb) and/or thulium (Tm). For example, iridium(III) bis(4,6-difluorophenylpyridinato-N,C2′)picolinate (FIrpic), bis(2,4-difluorophenylpyridinato)-tetrakis(1-pyrazolyl)borate iridium(III) (Fir6), or platinum octaethyl porphyrin (PtOEP) may be utilized as the phosphorescence dopant. However, one or more embodiments of the present disclosure is not limited thereto.

In some embodiments, the emission layer EML may include a hole transport host and an electron transport host. In some embodiments, the emission layer EML may include an auxiliary dopant and a light emitting dopant. In some embodiments, the auxiliary dopant may include a phosphorescence dopant material or a thermally activated delayed fluorescence dopant. For example, in one or more embodiments, the emission layer EML may include a hole transport host, an electron transport host, an auxiliary dopant, and a light emitting dopant.

In some embodiments, an exciplex may be formed by the hole transport host and the electron transport host in the emission layer EML. In this case, the triplet energy of the exciplex formed by the hole transport host and the electron transport host may correspond to T1 which is a gap between the LUMO energy level of the electron transport host and the HOMO energy level of the hole transport host.

In one or more embodiments, the triplet energy (T1) of the exiplex formed by the hole transport host and the electron transport host may be about 2.4 electron volts (eV) to about 3.0 eV. In some embodiments, the triplet energy of the exciplex may be a value smaller than the energy gap of each host material. Accordingly, the exciplex may have a triplet energy of about 3.0 eV or less, which is the energy gap between the hole transport host and the electron transport host.

In some embodiments, at least one emission layer EML may include a quantum dot material.

In the description, a quantum dot refers to the crystal of a semiconductor compound. The quantum dot may be to emit light in one or more suitable emission wavelengths according to the size of the crystal. The quantum dot may be to emit light in one or more suitable wavelengths by controlling the element ratio in the quantum dot compound. The diameter of the quantum dot may be, for example, about 1 nanometer (nm) to about 10 nm.

In one or more embodiments, the quantum dot may be synthesized by a chemical bath deposition, a metal organic chemical vapor deposition, a molecular beam epitaxy or a similar process therewith. For example, the chemical bath deposition is a method of mixing an organic solvent and a precursor material and then, growing quantum dot particle crystals. During growing the crystals, the organic solvent may naturally play the role of a dispersant that is coordinated with the quantum dot crystal surface, and may control the growth of the crystals. Accordingly, the chemical bath deposition is advantageous when compared to the metal organic chemical vapor deposition (MOCVD) or the molecular beam epitaxy (MBE), and may control the growth of quantum dot particles through a low-cost process.

The core of the quantum dot may be selected from among II-VI group compounds, I-II-IV group compounds, II-IV-VI group compounds, I-II-IV-VI group compound, III-VI group compounds, I-III-VI group compounds, III-V group compounds, III-II-V group compounds, IV-VI group compounds, IV group elements, IV group compounds, II-IV-V group compound, and/or combinations thereof.

The II-VI group compound may be selected from the group consisting of: a binary compound selected from the group consisting of CdSe, CdTe, CdS, ZnS, ZnSe, ZnTe, ZnO, HgS, HgSe, HgTe, MgSe, MgS, and mixtures thereof; a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and mixtures thereof; and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, and mixtures thereof. In some embodiments, the II-IV group semiconductor compound may further include I group metals and/or IV group elements. A I-II-IV group compound may be selected from CuSnS or CuZnS, and II-IV-VI group compound may select ZnSnS. A I-II-IV-VI group compound may be selected from a quaternary compound selected from the group consisting of Cu₂ZnSnS₂, Cu₂ZnSnS₄, Cu₂ZnSnSe₄, Ag₂ZnSnS₂ and mixtures thereof.

The Ill-VI group compound may include a binary compound such as GaS, GazS₃, GaSe, GazSe₃, GaTe, InS, InSe, In₂Se₃, InTe, and/or In₂Ss, a ternary compound such as InGaSs and/or InGaSes, or optional combination(s) thereof.

The I-III-VI group compound may be selected from a ternary compound selected from the group consisting of AgInS, AgInS₂, CuInS, CuInS₂, AgGaS₂, CuGaS₂, CuGaO₂, AgGaO₂, AgAlO₂, AgInSe₂, AgGaS, AgGaSe₂, CuInSe₂, CuGaSe₂, and mixtures thereof, or a quaternary compound such as AgInGaS₂, AgInGaSe₂, and CuInGaS₂.

The III-V group compound may be selected from the group consisting of a binary compound selected from the group consisting of GaN, GaP, GaAs, GaSb, AlN, AlP, AlAs, AlSb, InN, InP, InAs, InSb, and mixtures thereof, a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and mixtures thereof, and a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and mixtures thereof. In some embodiments, the III-V group compound may further include a II group metal. For example, InZnP, InGaZnP, InAlZnP, etc. may be included as a III-II-V group compound.

The IV-VI group compound may be selected from the group consisting of a binary compound selected from the group consisting of SnS, SnSe, SnTe, PbS, PbSe, PbTe, and mixtures thereof, a ternary compound selected from the group consisting of SnSeS, SnSeTe, SnSTe, PbSeS, PbSeTe, PbSTe, SnPbS, SnPbSe, SnPbTe, and mixtures thereof, and a quaternary compound selected from the group consisting of SnPbSSe, SnPbSeTe, SnPbSTe, and mixtures thereof. The IV group element may be selected from the group consisting of Si, Ge, and a mixture thereof. The IV group compound may be a binary compound selected from the group consisting of SiC, SiGe, and a mixture thereof.

The II-IV-V group compound may be a ternary compound selected from the group consisting of ZnSnP, ZnSnP₂, ZnSnAs₂, ZnGeP₂, ZnGeAs₂, CdSnP₂, and CdGeP₂, and mixtures thereof.

Each element included in a multi-element compound such as the binary compound, the ternary compound and the quaternary compound may be present at substantially uniform concentration or non-substantially uniform concentration in a particle. For example, the chemical formulas may refer to the type or kind of elements included in the compound, and the element ratio in the compound may be different. For example, AgInGaS₂ may refer to AgInxGa₁₋ₓS₂ (x is a real number between 0 and 1).

In some embodiments, the quantum dot of one or more embodiments may have a core/shell structure in which one quantum dot wraps another quantum dot. In the core/shell structure, the concentration of an element present in the shell may have a concentration gradient which is decreased toward the center.

In some embodiments, the quantum dot may have the described core-shell structure including a core including a nanocrystal and a shell wrapping the core. The shell of the quantum dot may play the role of a protection layer for preventing or reducing the chemical deformation of the core to maintain semiconductor properties and/or a charging layer for imparting the quantum dot with electrophoretic properties. The shell may have a single layer or a multilayer. Examples of the shell of the quantum dot may include a metal or non-metal oxide, a semiconductor compound, or combinations thereof.

For example, the metal or non-metal oxide may include a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄ and/or NiO, or a ternary compound such as MgAl₂O₄, CoFe₂O₄, NiFe₂O₄ and/or CoMn₂O₄, but one or more embodiments of the present disclosure is not limited thereto.

Also, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, and/or the like, but one or more embodiments of the present disclosure is not limited thereto.

The quantum dot may have a full width of half maximum (FWHM) of emission wavelength spectrum of about 45 nm or less, about 40 nm or less, more, about 30 nm or less. Within this range, color purity and/or color reproducibility may be enhanced or improved. In some embodiments, light emitted via such quantum dot is emitted in all directions, and light view angle properties may be enhanced or improved, e.g., the size or width of the viewing angle of a display device and/or light emitting element having the quantum dot as described herein may be increased.

In some embodiments, the shape of the quantum dot may be generally utilized shapes in the art, without specific limitation. In some embodiments, the shape of spherical, pyramidal, multi-arm, or cubic nanoparticle, nanotube, nanowire, nanofiber, nanoplate particle, etc. may be utilized.

The quantum dot may determine or control the color of light emitted according to the particle size, and accordingly, the quantum dot may have one or more suitable emission colors such as blue, red, green, and/or the like.

An energy band gap may be controlled or selected by controlling the particle size or by controlling the element ratio in the quantum dot compound. Accordingly, the quantum dot may be to emit light in one or more suitable wavelength bands. In one or more embodiments, a light emitting element emitting light with one or more suitable wavelengths may be accomplished by utilizing quantum dots having different sizes from each other or by utilizing quantum dots of which element ratio in the quantum dot compound has been controlled or selected. For example, the control of the size of the quantum dot or the element ratio in the quantum dot compound may be selected so as to emit red, green and/or blue light. In some embodiments, the quantum dots may be constituted so as to combine one or more suitable colors of light to emit white light.

In the light emitting element(s) ED of one or more embodiments, as shown in FIG. 3 to FIG. 6, the electron transport region ETR is provided on the emission layer EML. The electron transport region ETR may include at least one of a hole blocking layer HBL, an electron transport layer ETL or an electron injection layer EIL. However, one or more embodiments of the present disclosure is not limited thereto.

The electron transport region ETR may have a single layer formed utilizing a single material, a single layer formed utilizing multiple different materials, or a multilayer structure having multiple layers formed utilizing multiple different materials.

For example, the electron transport region ETR may have a single layer structure of an electron injection layer EIL or an electron transport layer ETL, or a single layer structure formed utilizing an electron injection material and an electron transport material. Further, the electron transport region ETR may have a single layer structure formed utilizing multiple different materials, and/or a structure stacked from the emission layer EML of electron transport layer ETL/electron injection layer EIL, hole blocking layer HBL/electron transport layer ETL/electron injection layer EIL, electron transport layer ETL/buffer layer/electron injection layer EIL, without limitation. The thickness of the electron transport region ETR may be, for example, from about 1,000 Å to about 1,500 Å.

The electron transport region ETR may be formed utilizing one or more suitable methods such as a vacuum deposition method, a spin coating method, a cast method, a Langmuir-Blodgett (LB) method, an inkjet printing method, a laser printing method, and/or a laser induced thermal imaging (LITI) method.

The electron transport region ETR may include a compound represented by Formula ET-1.

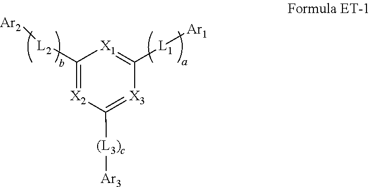

In Formula ET-1, at least one among X₁ to X₃ is N, and the remainder are CRₐ. Each Rₐ may independently be a hydrogen atom, a deuterium atom, a substituted or unsubstituted alkyl of 1 to 20 carbon atoms, a substituted or unsubstituted aryl group of 6 to 30 ring-forming carbon atoms, or a substituted or unsubstituted heteroaryl group of 2 to 30 ring-forming carbon atoms. Ar₁ to Ar₃ may each independently be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In Formula ET-1, “a” to “c” may each independently be an integer of 0 to 10. In Formula ET-1, L₁ to L₃ may each independently be a direct linkage, a substituted or unsubstituted arylene group of 6 to 30 ring-forming carbon atoms, or a substituted or unsubstituted heteroarylene group of 2 to 30 ring-forming carbon atoms. In some embodiments, when “a” to “c” are integers of 2 or more, L₁ to L₃ may each independently be a substituted or unsubstituted arylene group of 6 to 30 ring-forming carbon atoms, or a substituted or unsubstituted heteroarylene group of 2 to 30 ring-forming carbon atoms.

The electron transport region ETR may include an anthracene-based compound. However, one or more embodiments of the present disclosure is not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazol-1-ylphenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), berylliumbis(benzoquinolin-10-olate (Bebq2), 9,10-di(naphthalen-2-yl)anthracene (ADN), 1,3-bis[3,5-di(pyridin-3-yl)phenyl]benzene (BmPyPhB), diphenyl(4-(triphenylsilyl)phenyl)phosphine oxide (TSPO1), and/or mixture(s) thereof, without limitation.

The electron transport region ETR may include at least one selected from among Compounds ET1 to ET36.

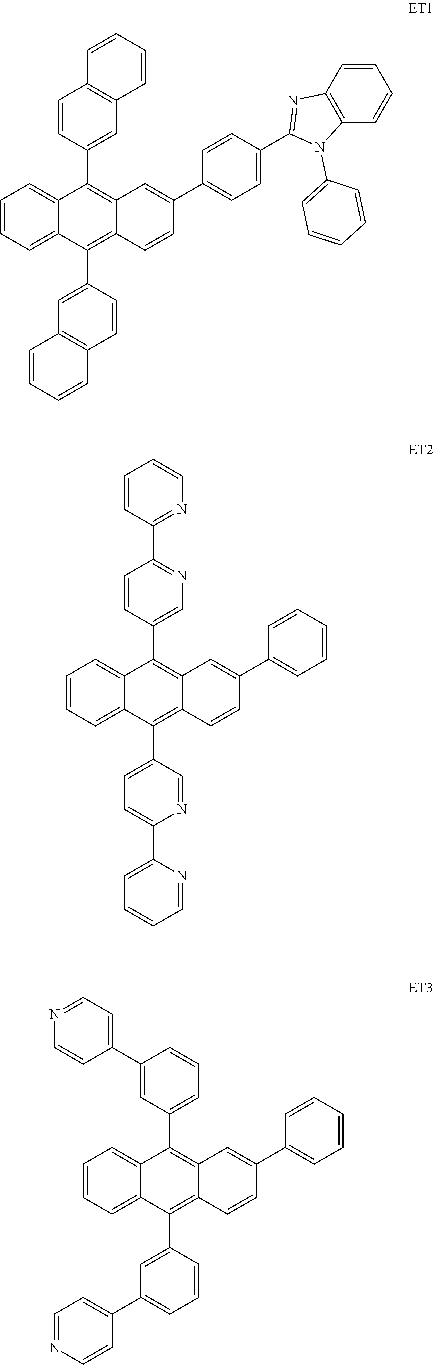 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

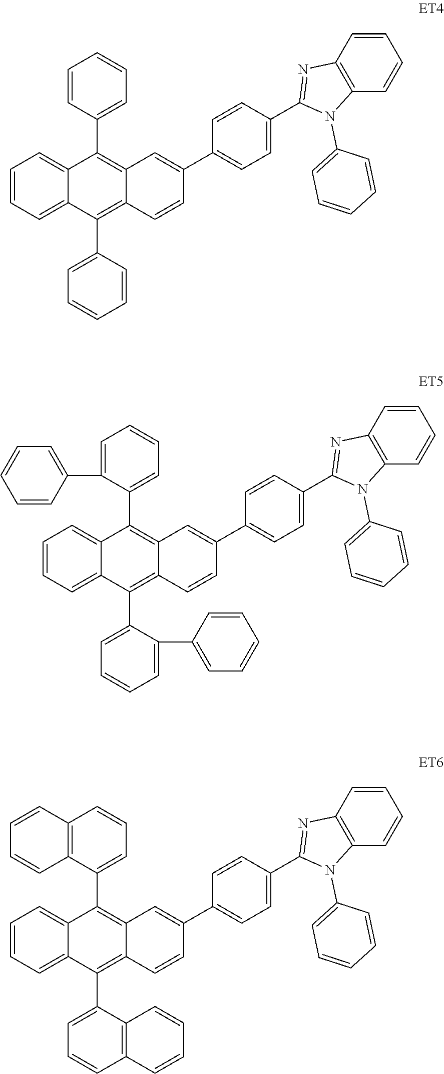 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

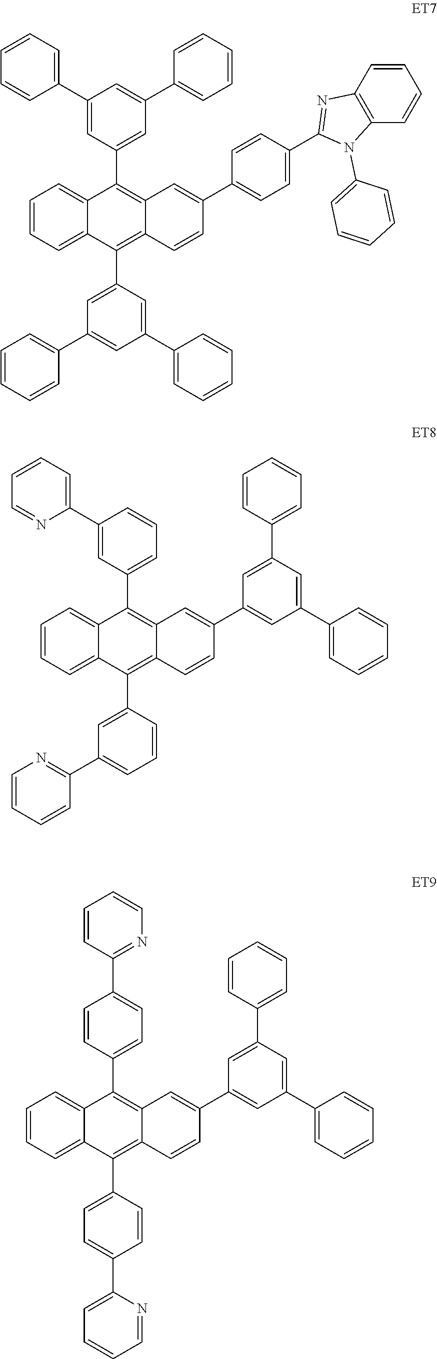 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

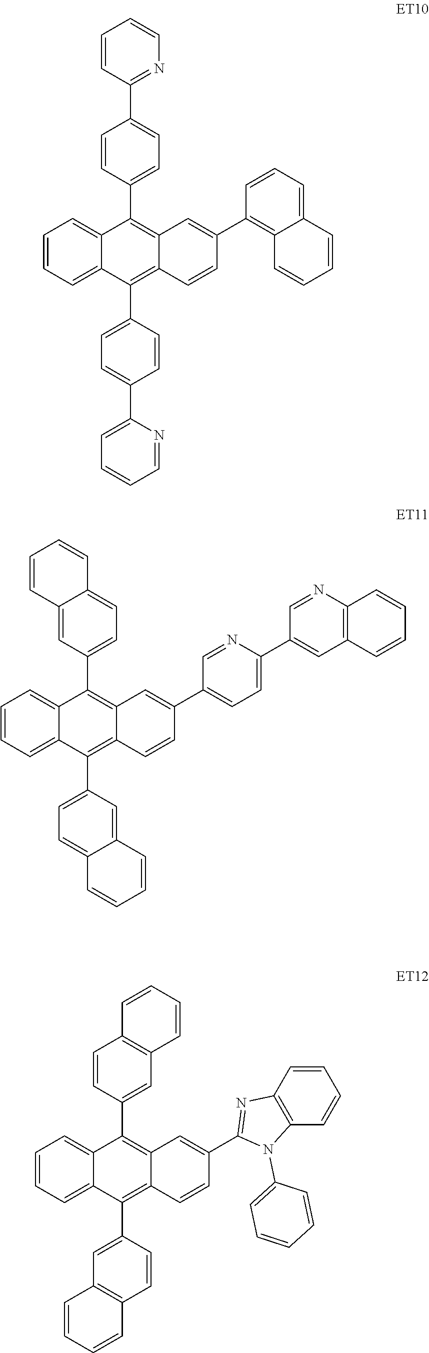 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

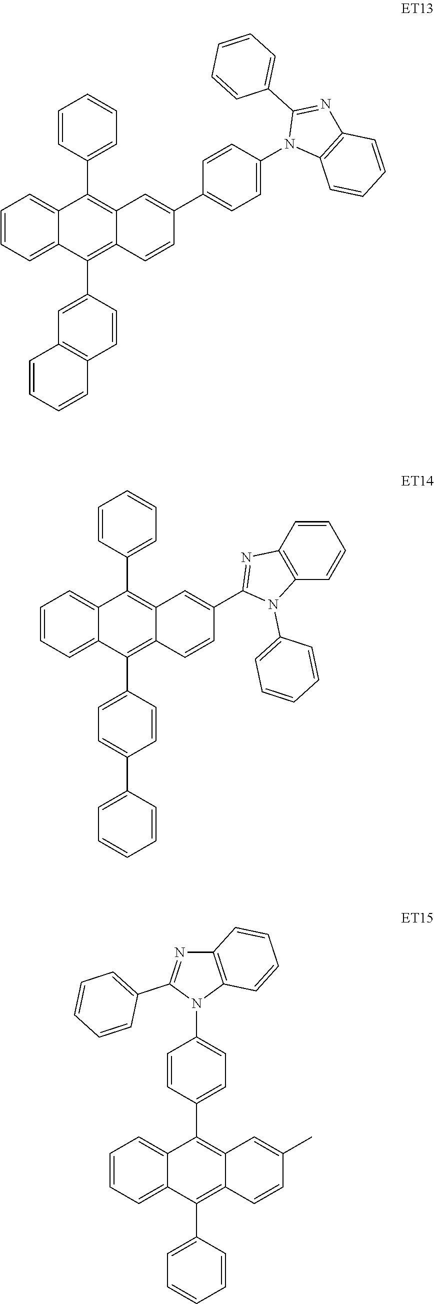 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

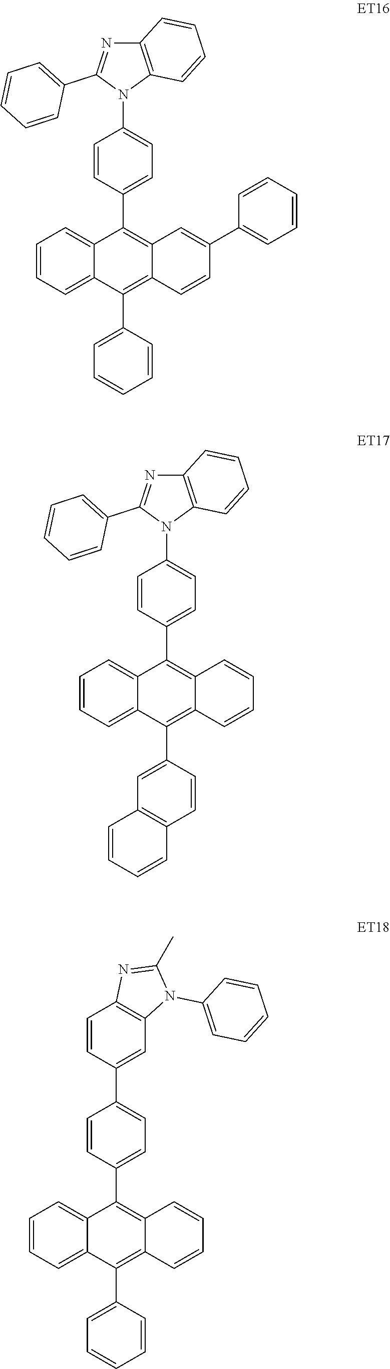 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

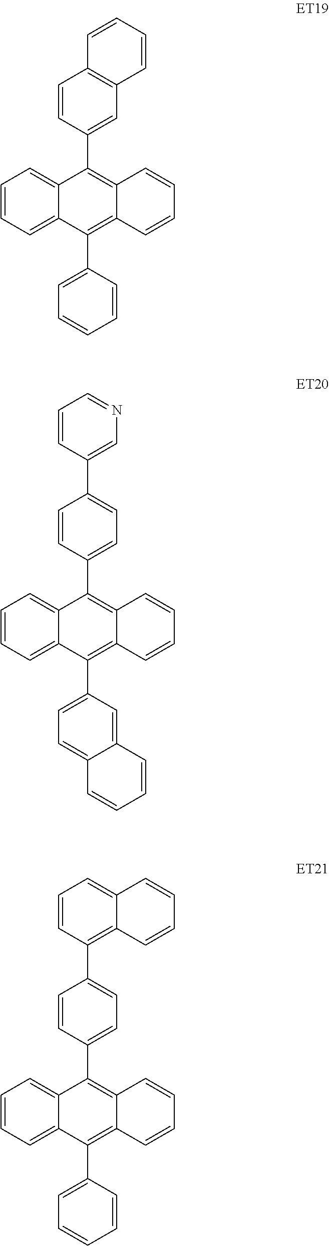

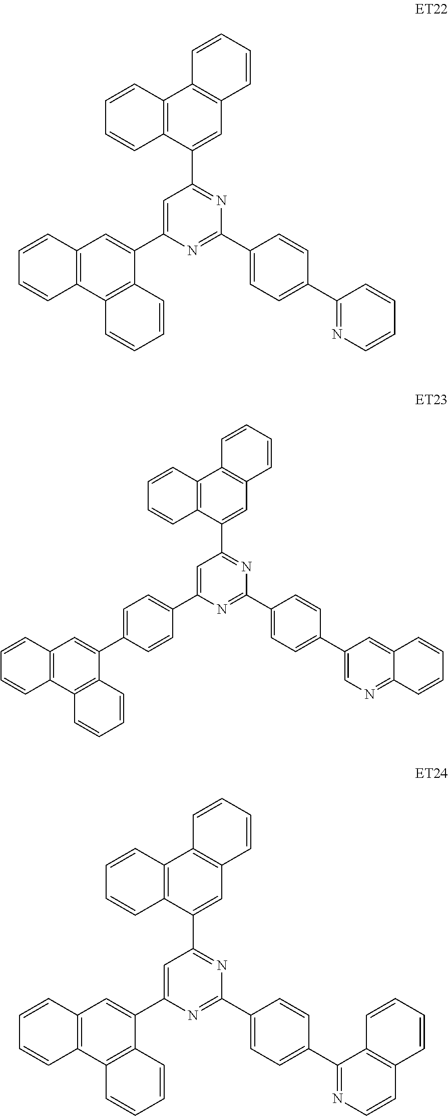 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

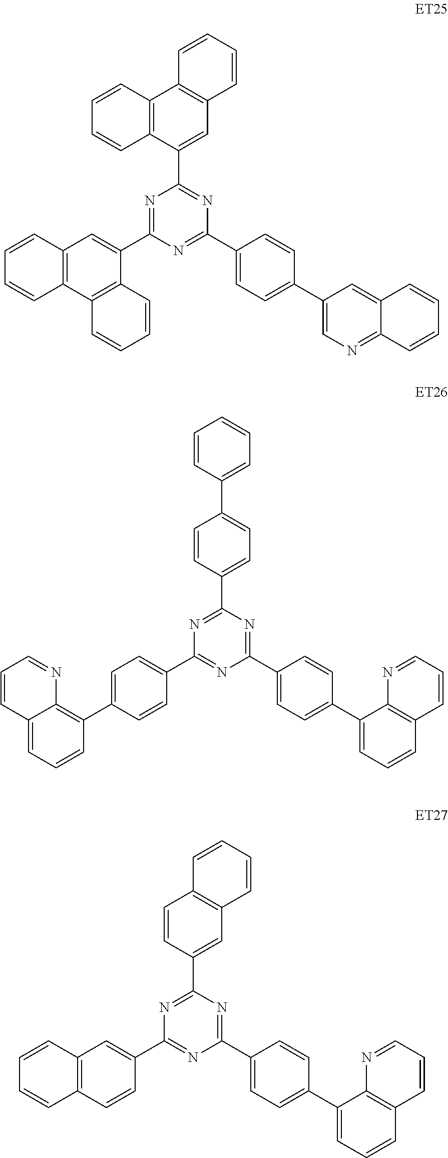 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

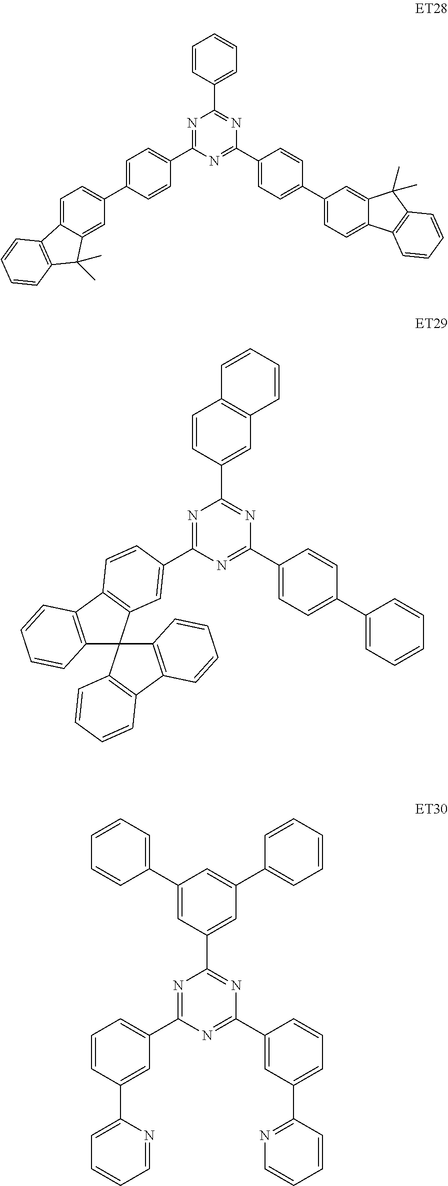 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3nc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

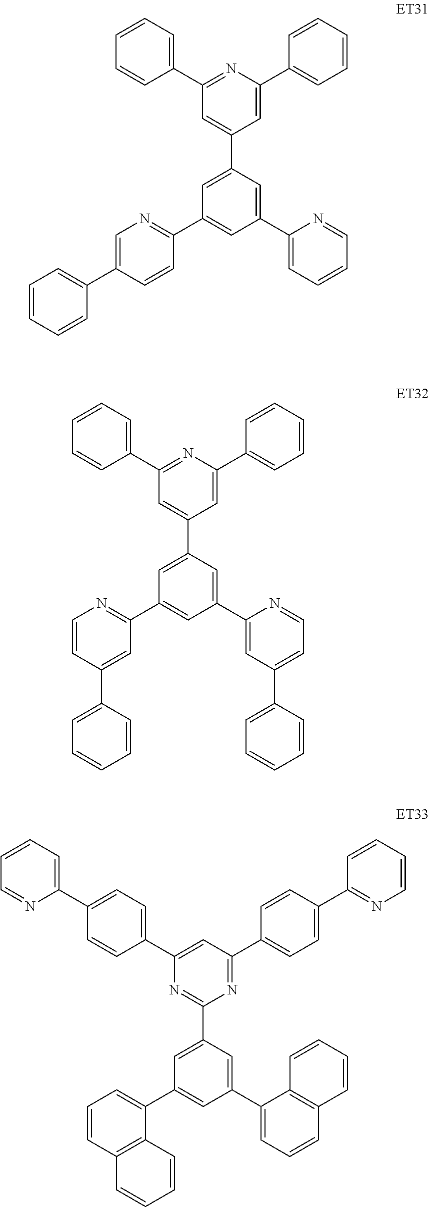 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

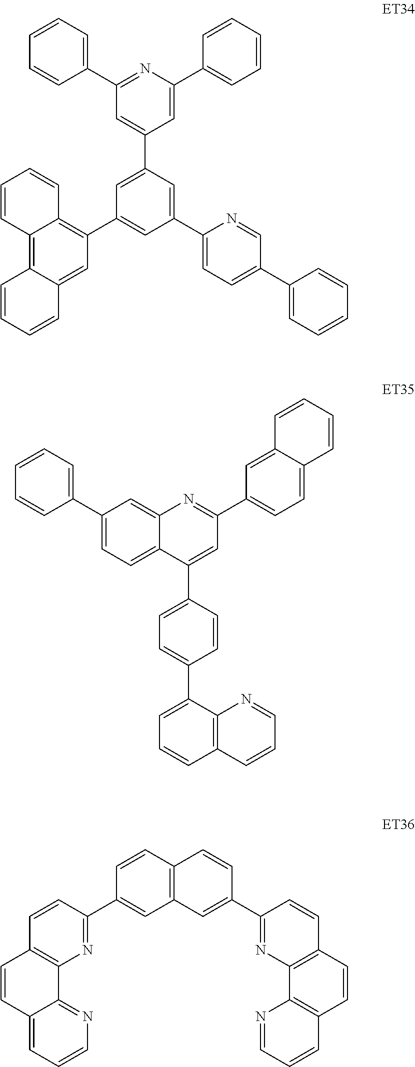 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In some embodiments, the electron transport region ETR may include a metal halide such as LiF, NaCl, CsF, RbCl, RbI, CuI, KI, and/or the like, a lanthanide metal such as Yb, or a co-depositing material of the metal halide and the lanthanide metal. For example, the electron transport region ETR may include KI:Yb, RbI:Yb, LIF:Yb, and/or the like, as the co-depositing material. In some embodiments, the electron transport region ETR may utilize a metal oxide such as Li₂O, BaO, and/or the like, or 8-hydroxy-lithium quinolate (Liq). However, one or more embodiments of the present disclosure is not limited thereto. The electron transport region ETR also may be formed utilizing a mixture material of an electron transport material and an insulating organo metal salt. The organo metal salt may be a material having an energy band gap of about 4 eV or more. For example, the organo metal salt may include, for example, metal acetates, metal benzoates, metal acetoacetates, metal acetylacetonates, metal stearates, and/or the like.

The electron transport region ETR may include at least one of 2,9-dimethyl-4,7-diphenyl-1,10-phenanthroline (BCP), diphenyl(4-(triphenylsilyl)phenyl)phosphine oxide (TSPO1) or 4,7-diphenyl-1,10-phenanthroline (Bphen) in addition to the aforementioned materials. However, one or more embodiments of the present disclosure is not limited thereto.

The electron transport region ETR may include the compounds of the electron transport region in at least one among an electron injection layer EIL, an electron transport layer ETL, and a hole blocking layer HBL.

When the electron transport region ETR includes the electron transport layer ETL, the thickness of the electron transport layer ETL may be from about 100 Å to about 1,000 Å, for example, from about 150 Å to about 500 Å. When the thickness of the electron transport layer ETL satisfies the described range, satisfactory electron transport properties may be obtained without substantial increase of a driving voltage. When the electron transport region ETR includes the electron injection layer EIL, the thickness of the electron injection layer EIL may be from about 1 Å to about 100 Å, and from about 3 Å to about 90 Å. When the thickness of the electron injection layer EIL satisfies the described range, satisfactory electron injection properties may be obtained without inducing substantial increase of a driving voltage.

The second electrode EL2 is provided on the electron transport region ETR. The second electrode EL2 may be a common electrode. The second electrode EL2 may be a cathode or an anode, but one or more embodiments of the present disclosure is not limited thereto. For example, when the first electrode EL1 is an anode, the second cathode EL2 may be a cathode, and when the first electrode EL1 is a cathode, the second electrode EL2 may be an anode. The second electrode EL2 may include at least one selected among Ag, Mg, Cu, Al, Pt, Pd, Au, Ni, Nd, Ir, Cr, Li, Ca, LiF, Mo, Ti, W, In, Sn, Zn, compounds of two or more selected therefrom, mixtures of two or more selected therefrom, or oxides thereof.

The second electrode EL2 may be a transmissive electrode, a transflective electrode or a reflective electrode. When the second electrode EL2 is the transmissive electrode, the second electrode EL2 may include a transparent metal oxide, for example, ITO, IZO, ZnO, ITZO, and/or the like.

When the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca (stacked structure of LiF and Ca), LiF/Al (stacked structure of LiF and Al), Mo, Ti, Yb, W, compound(s) including thereof, or mixture(s) thereof (for example, AgMg, AgYb, or MgYb). In some embodiments, the second electrode EL2 may have a multilayered structure including a reflective layer or a transflective layer formed utilizing the above-described materials and a transparent conductive layer formed utilizing ITO, IZO, ZnO, ITZO, etc. For example, the second electrode EL2 may include the aforementioned metal material(s), combination(s) of two or more metal materials selected from the aforementioned metal materials, or oxide(s) of the aforementioned metal materials.

In some embodiments, the second electrode EL2 may be connected to or with an auxiliary electrode. When the second electrode EL2 is connected to or with the auxiliary electrode, the resistance of the second electrode EL2 may decrease.

In some embodiments, on the second electrode EL2 in the light emitting element ED of one or more embodiments, a capping layer CPL may be further provided. The capping layer CPL may include a multilayer or a single layer.

In one or more embodiments, the capping layer CPL may be an organic layer or an inorganic layer. For example, when the capping layer CPL includes an inorganic material, the inorganic material may include an alkali metal compound such as LiF, an alkaline earth metal compound such as SiON, SiNx, SiOy, and/or the like.

For example, when the capping layer CPL includes an organic material, the organic material may include α-NPD, NPB, TPD, m-MTDATA, Alq₃, CuPc, N4,N4,N4′, N4′-tetra(biphenyl-4-yl) biphenyl-4,4′-diamine (TPD15), 4,4′,4″-tris(carbazol-1 9-yl) triphenylamine (TCTA), and/or the like, or includes an epoxy resin, and/or acrylate such as methacrylate. In some embodiments, a capping layer CPL may include at least one selected from among Compounds P1 to P5, but one or more embodiments of the present disclosure is not limited thereto.

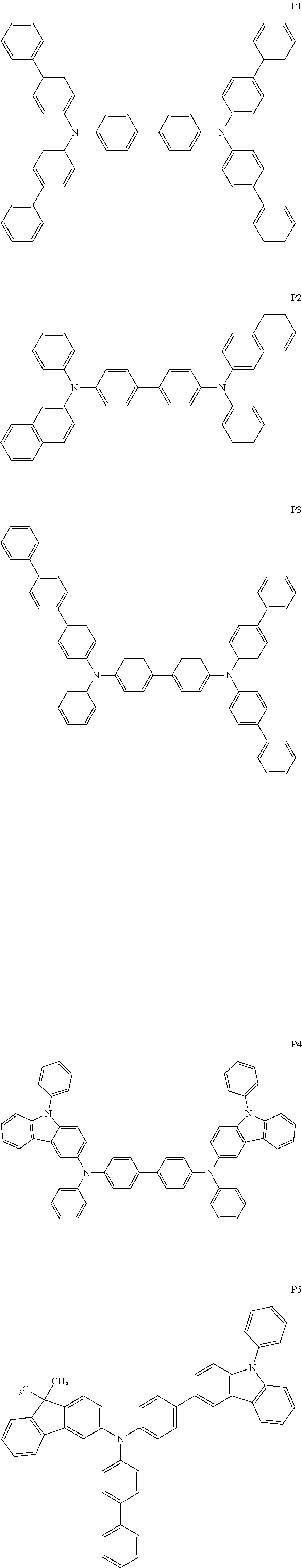 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

In some embodiments, the refractive index of the capping layer CPL may be about 1.6 or more. For example, the refractive index of the capping layer CPL with respect to light in a wavelength range of about 550 nm to about 660 nm may be about 1.6 or more.

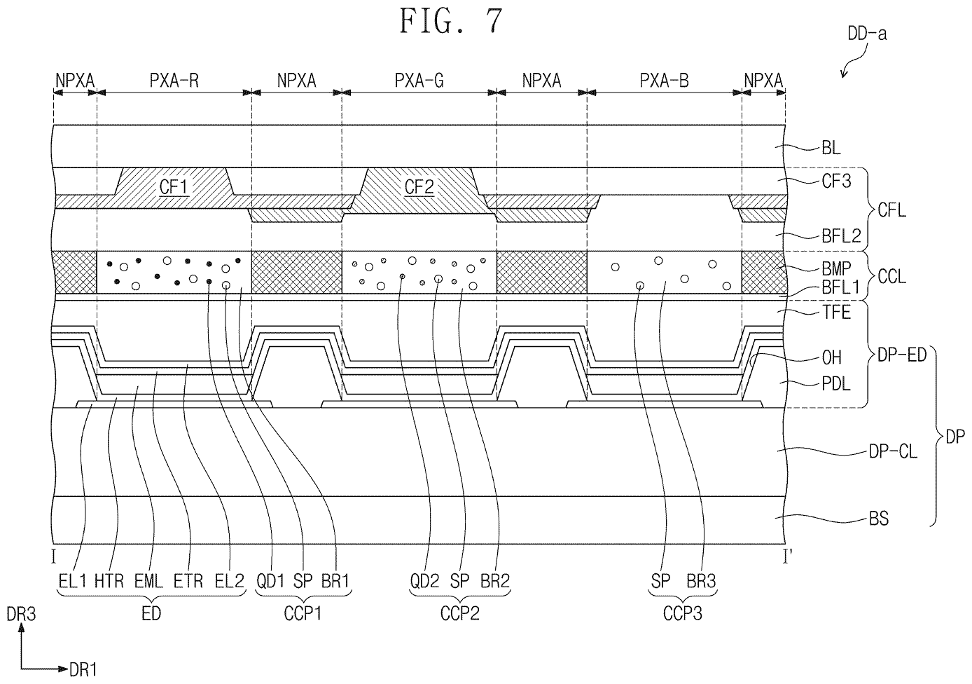

FIG. 7 to FIG. 10 are each a cross-sectional view of a display device according to one or more embodiments. Hereinafter, in the explanation of the display devices of one or more embodiments, referring to FIG. 7 to FIG. 10, the overlapping parts with the explanation on FIG. 1 to FIG. 6 will not be explained again, and the different features will be explained chiefly.

Referring to FIG. 7, a display device DD-a according to one or more embodiments may include a display panel DP including a display element layer DP-ED, a light controlling layer CCL provided on the display panel DP, and a color filter layer CFL.

In one or more embodiments shown in FIG. 7, the display panel DP includes a base layer BS, a circuit layer DP-CL provided on the base layer BS and a display element layer DP-ED, and the display element layer DP-ED may include a light emitting element ED.

The light emitting element ED may include a first electrode EL1, a hole transport region HTR provided on the first electrode EL1, an emission layer EML provided on the hole transport region HTR, an electron transport region ETR provided on the emission layer EML, and a second electrode EL2 provided on the electron transport region ETR. In some embodiments, the structures of the light emitting elements of FIG. 3 to FIG. 6 may be applied to the structure of the light emitting element ED shown in FIG. 7.

The hole transport region HTR of the light emitting element ED included in the display device DD-a according to one or more embodiments may include the amine compound of one or more embodiments.

Referring to FIG. 7, the emission layer EML may be provided in an opening portion OH defined in a pixel definition layer PDL. For example, the emission layer EML that is divided by the pixel definition layer PDL and correspondingly provided to each of luminous areas PXA-R, PXA-G and PXA-B may be to emit light in substantially the same wavelength region. In the display device DD-a of one or more embodiments, the emission layer EML may be to emit blue light. In some embodiments, different from the drawings, in one or more embodiments, the emission layer EML may be provided as a common layer for all luminous areas PXA-R, PXA-G and PXA-B.

The light controlling layer CCL may be provided on the display panel DP. The light controlling layer CCL may include a light converter. The light converter may be a quantum dot or a phosphor. The light converter may transform the wavelength of light provided and then emit. For example, the light controlling layer CCL may be a layer including a quantum dot or a layer including a phosphor.

The light controlling layer CCL may include multiple light controlling parts CCP1, CCP2 and CCP3. The light controlling parts CCP1, CCP2 and CCP3 may be separated from one another.

Referring to FIG. 7, a partition pattern BMP may be provided between the separated light controlling parts CCP1, CCP2 and CCP3, but one or more embodiments of the present disclosure is not limited thereto. In FIG. 7, the partition pattern BMP is shown not to be overlapped with the light controlling parts CCP1, CCP2 and CCP3, but at least a portion of the edge of the light controlling parts CCP1, CCP2 and CCP3 may be overlapped with the partition pattern BMP.

The light controlling layer CCL may include a first light controlling part CCP1 including a first quantum dot QD1 converting first color light provided from the light emitting element ED into second color light, a second light controlling part CCP2 including a second quantum dot QD2 converting first color light into third color light, and a third light controlling part CCP3 transmitting first color light.

In one or more embodiments, the first light controlling part CCP1 may provide red light which is the second color light, and the second light controlling part CCP2 may provide green light which is the third color light. The third light controlling part CCP3 may be to transmit and provide blue light which is the first color light provided from the light emitting element ED. For example, the first quantum dot QD1 may be a red quantum dot, and the second quantum dot QD2 may be a green quantum dot. On the quantum dots QD1 and QD2, the same contents as those described herein may be applied.

In some embodiments, the light controlling layer CCL may further include a scatterer SP. The first light controlling part CCP1 may include the first quantum dot QD1 and the scatterer SP, the second light controlling part CCP2 may include the second quantum dot QD2 and the scatterer SP, and the third light controlling part CCP3 may not include (e.g., may exclude) a (any) quantum dot but include the scatterer SP.

The scatterer SP may be an inorganic particle. For example, the scatterer SP may include at least one selected from among TiO₂, ZnO, Al₂O₃, SiO₂, and hollow silica. The scatterer SP may include at least one selected from among TiO₂, ZnO, Al₂O₃, SiO₂, and hollow silica, or may be a mixture of two or more materials selected among TiO₂, ZnO, Al₂O₃, SiO₂, and hollow silica.

Each of the first light controlling part CCP1, the second light controlling part CCP2, and the third light controlling part CCP3 may include base resins BR1, BR2 and BR3 dispersing the quantum dots QD1 and QD2 and the scatterer SP. In one or more embodiments, the first light controlling part CCP1 may include the first quantum dot QD1 and the scatterer SP dispersed in the first base resin BR1, the second light controlling part CCP2 may include the second quantum dot QD2 and the scatterer SP dispersed in the second base resin BR2, and the third light controlling part CCP3 may include the scatterer SP dispersed in the third base resin BR3. The base resins BR1, BR2 and BR3 are mediums in which the quantum dots QD1 and QD2 and the scatterer SP are dispersed, and may be composed of one or more suitable resin compositions which may be generally referred to as a binder. For example, the base resins BR1, BR2 and BR3 may be acrylic-based resins, urethane-based resins, silicone-based resins, epoxy-based resins, etc. The base resins BR1, BR2 and BR3 may be transparent resins. In one or more embodiments, the first base resin BR1, the second base resin BR2 and the third base resin BR3 may be the same or different from each other.

The light controlling layer CCL may include a barrier layer BFL1. The barrier layer BFL1 may play the role of blocking the penetration of moisture and/or oxygen (hereinafter, will be referred to as “humidity/oxygen”). The barrier layer BFL1 may be provided on the light controlling parts CCP1, CCP2 and CCP3 and may block or reduce the exposure of the light controlling parts CCP1, CCP2 and CCP3 to humidity/oxygen. In some embodiments, the barrier layer BFL1 may cover the light controlling parts CCP1, CCP2 and CCP3. In some embodiments, the barrier layer BFL2 may be provided between the light controlling parts CCP1, CCP2 and CCP3 and a color filter layer CFL.

The barrier layers BFL1 and BFL2 may include at least one inorganic layer. For example, the barrier layers BFL1 and BFL2 may be formed by including an inorganic material. For example, the barrier layers BFL1 and BFL2 may be formed by including silicon nitride, aluminum nitride, zirconium nitride, titanium nitride, hafnium nitride, tantalum nitride, silicon oxide, aluminum oxide, titanium oxide, tin oxide, cerium oxide and/or silicon oxynitride, or a metal thin film securing light transmittance. In some embodiments, the barrier layers BFL1 and BFL2 may further include an organic layer. The barrier layers BFL1 and BFL2 may include or be composed of a single layer or multiple layers.

In the display device DD-a of one or more embodiments, the color filter layer CFL may be provided on the light controlling layer CCL. For example, the color filter layer CFL may be provided directly on the light controlling layer CCL. In this case, the barrier layer BFL2 may not be provided.

The color filter layer CFL may include filters CF1, CF2 and CF3. The color filter layer CFL may include a first filter CF1 transmitting second color light, a second filter CF2 transmitting third color light, and a third filter CF3 transmitting first color light. For example, the first filter CF1 may be a red filter, the second filter CF2 may be a green filter, and the third filter CF3 may be a blue filter. Each of the filters CF1, CF2 and CF3 may include a polymer photosensitive resin and/or a pigment and/or dye. The first filter CF1 may include a red pigment and/or dye, the second filter CF2 may include a green pigment and/or dye, and the third filter CF3 may include a blue pigment and/or dye. In some embodiments, one or more embodiments of the present disclosure is not limited thereto, and the third filter CF3 may not include (e.g., may exclude e.g., may exclude any of)) the pigment and/or dye. The third filter CF3 may include a polymer photosensitive resin and not include a (any) pigment and/or dye. The third filter CF3 may be transparent. The third filter CF3 may be formed utilizing a transparent photosensitive resin.

In some embodiments, in one or more embodiments, the first filter CF1 and/or the second filter CF2 may be yellow filters. The first filter CF1 and/or the second filter CF2 may be provided in one body without distinction. The first to third filters CF1, CF2 and CF3 may be provided corresponding to the red luminous area PXA-R, the green luminous area PXA-G and the blue luminous area PXA-B, respectively.

In some embodiments, though not shown, the color filter layer CFL may further include a light blocking part. The color filter layer CFL may include a light blocking part provided at the boundaries to overlap with adjacent filters CF1, CF2 and CF3. The light blocking part may be a black matrix. The light blocking part may be formed by including an organic light blocking material or an inorganic light blocking material, including a black pigment and/or black dye. The light blocking part may divide or separate the boundaries among adjacent filters CF1, CF2 and CF3. In one or more embodiments, the light blocking part may be formed as a blue filter.

On the color filter layer CFL, a base substrate BL may be provided. The base substrate BL may be a member providing a base surface on which the color filter layer CFL, the light controlling layer CCL, etc. are provided. The base substrate BL may be a glass substrate, a metal substrate, a plastic substrate, etc. However, one or more embodiments of the present disclosure is not limited thereto, and the base substrate BL may be an inorganic layer, an organic layer or a composite material layer. In some embodiments, different from the drawing, the base substrate BL may not be provided.

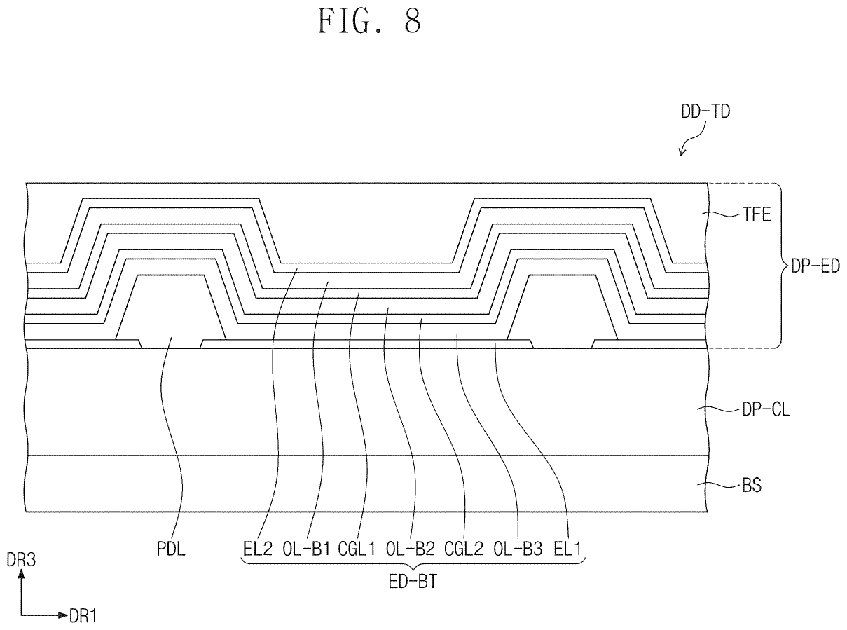

Referring to FIG. 8, a display device DD-TD of one or more embodiments includes the light emitting element ED-BT that may include multiple light emitting structures OL-B1, OL-B2 and OL-B3. The light emitting element ED-BT may include oppositely provided first electrode EL1 and second electrode EL2, and the multiple light emitting structures OL-B1, OL-B2 and OL-B3 stacked in order in a thickness direction and provided between the first electrode EL1 and the second electrode EL2. Each of the light emitting structures OL-B1, OL-B2 and OL-B3 may include the emission layer EML (FIG. 7), and a hole transport region HTR (FIG. 7) and an electron transport region ETR (FIG. 7), provided with the emission layer EML therebetween.

For example, the light emitting element ED-BT included in the display device DD-TD of one or more embodiments may be a light emitting element of a tandem structure including multiple emission layers EML.

In one or more embodiments shown in FIG. 8, light emitted from the light emitting structures OL-B1, OL-B2 and OL-B3 may be all blue light. However, one or more embodiments of the present disclosure is not limited thereto, and the wavelength regions of light emitted from the light emitting structures OL-B1, OL-B2 and OL-B3 may be different from each other. For example, the light emitting element ED-BT including the multiple light emitting structures OL-B1, OL-B2 and OL-B3 emitting light in different wavelength regions may be to emit white light.

Between neighboring light emitting structures OL-B1, OL-B2 and OL-B3, charge generating layers CGL1 and CGL2 may be provided. The charge generating layers CGL1 and CGL2 may include a p-type or kind charge generating layer and/or an n-type or kind charge generating layer.

In at least one among the light emitting structures OL-B1, OL-B2 and OL-B3, included in the display device DD-TD of one or more embodiments, the amine compound of one or more embodiments may be included.

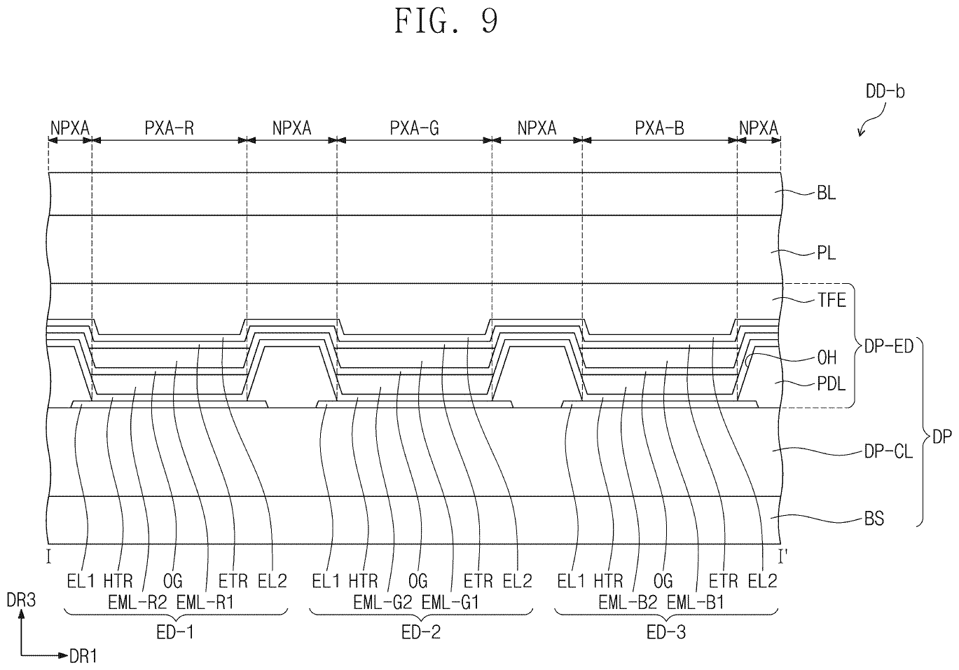

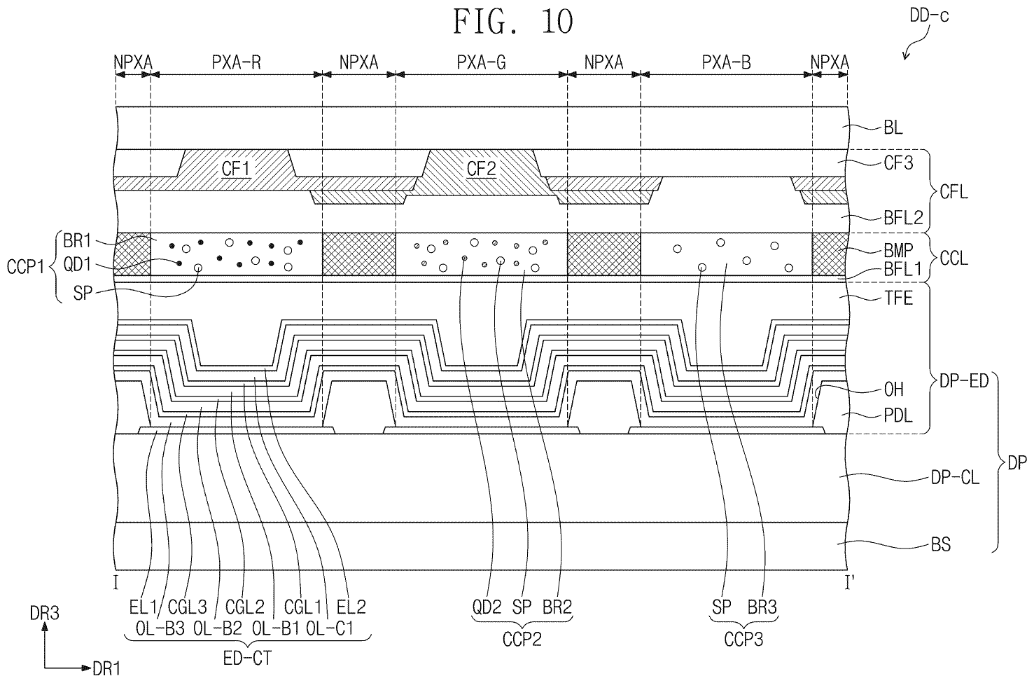

Referring to FIG. 9, a display device DD-b according to one or more embodiments may include light emitting elements ED-1, ED-2 and ED-3, formed by stacking two emission layers. Compared to the display device DD of one or more embodiments shown in FIG. 2, one or more embodiments shown in FIG. 9 is different in that first to third light emitting elements ED-1, ED-2 and ED-3 include two emission layers stacked in a thickness direction, each. In the first to third light emitting elements ED-1, ED-2 and ED-3, two emission layers may be to emit light in substantially the same wavelength region.

The first light emitting element ED-1 may include a first red emission layer EML-R1 and a second red emission layer EML-R2. The second light emitting element ED-2 may include a first green emission layer EML-G1 and a second green emission layer EML-G2. In some embodiments, the third light emitting element ED-3 may include a first blue emission layer EML-B1 and a second blue emission layer EML-B2. Between the first red emission layer EML-R₁ and the second red emission layer EML-R₂, between the first green emission layer EML-G1 and the second green emission layer EML-G2, and/or between the first blue emission layer EML-B1 and the second blue emission layer EML-B2, an emission auxiliary part OG may be provided.

The emission auxiliary part OG may include a single layer or a multilayer. The emission auxiliary part OG may include a charge generating layer. More particularly, the emission auxiliary part OG may include an electron transport region, a charge generating layer, and a hole transport region stacked in order. The emission auxiliary part OG may be provided as a common layer in all of the first to third light emitting elements ED-1, ED-2 and ED-3. However, one or more embodiments of the present disclosure is not limited thereto, and the emission auxiliary part OG may be patterned and provided in an opening portion OH defined in a pixel definition layer PDL.

The first red emission layer EML-R1, the first green emission layer EML-G1 and/or the first blue emission layer EML-B1 may be provided between the electron transport region ETR and the emission auxiliary part OG. The second red emission layer EML-R₂, the second green emission layer EML-G2 and the second blue emission layer EML-B2 may be provided between the emission auxiliary part OG and the hole transport region HTR.

For example, the first light emitting element ED-1 may include a first electrode EL1, a hole transport region HTR, a second red emission layer EML-R₂, an emission auxiliary part OG, a first red emission layer EML-R₁, an electron transport region ETR, and a second electrode EL2, stacked in order. The second light emitting element ED-2 may include a first electrode EL1, a hole transport region HTR, a second green emission layer EML-G2, an emission auxiliary part OG, a first green emission layer EML-G1, an electron transport region ETR, and a second electrode EL2, stacked in order. The third light emitting element ED-3 may include a first electrode EL1, a hole transport region HTR, a second blue emission layer EML-B2, an emission auxiliary part OG, a first blue emission layer EML-B1, an electron transport region ETR, and a second electrode EL2, stacked in order.

In some embodiments, an optical auxiliary layer PL may be provided on a display element layer DP-ED. The optical auxiliary layer PL may include a polarization layer. The optical auxiliary layer PL may be provided on a display panel DP and may control reflected light at the display panel DP by external light. Different from the drawings, the optical auxiliary layer PL may not be provided from the display device according to one or more embodiments.

Referring to FIG. 10, and different from FIG. 8 and FIG. 9, a display device DD-c is shown to include four light emitting structures OL-B1, OL-B2, OL-B3 and OL-C1. A light emitting element ED-CT may include oppositely provided first electrode EL1 and second electrode EL2, and first to fourth light emitting structures OL-B1, OL-B2, OL-B3 and OL-C1 stacked in order in a thickness direction between the first electrode EL1 and the second electrode EL2. Between the first to fourth light emitting structures OL-B1, OL-B2, OL-B3 and OL-C1, charge generating layers CGL1, CGL2 and CGL3 may be provided. Among the four light emitting structures, the first to third light emitting structures OL-B1, OL-B2 and OL-B3 may be to emit blue light, and the fourth light emitting structure OL-C1 may be to emit green light. However, one or more embodiments of the present disclosure is not limited thereto, and the first to fourth light emitting structures OL-B1, OL-B2, OL-B3 and OL-C1 may be to emit different wavelengths of light.

Charge generating layers CGL1, CGL2 and CGL3 provided among neighboring light emitting structures OL-B1, OL-B2, OL-B3 and OL-C1 may include a p-type or kind charge generating layer and/or an n-type or kind charge generating layer.

In at least one among the light emitting structures OL-B1, OL-B2, OL-B3 and OL-C1, included in the display device DD-c of one or more embodiments, the amine compound of one or more embodiments may be included.

The light emitting element ED according to one or more embodiments of the present disclosure may include the amine compound of one or more embodiments in at least one functional layer provided between the first electrode EL1 and the second electrode EL2 to show enhanced or improved emission efficiency and improved life characteristics. The light emitting element ED according to one or more embodiments may include the amine compound of one or more embodiments in at least one among a hole transport region HTR, an emission layer EML, and an electron transport region ETR, provided between the first electrode EL1 and the second electrode EL2, or in a capping layer CPL. For example, the amine compound according to one or more embodiments may be included in the hole transport region HTR of the light emitting element ED of one or more embodiments, and the light emitting element of one or more embodiments may show high emission efficiency and long-life (e.g., long lifetime) characteristics.

The amine compound of one or more embodiments includes a core nitrogen atom connected to a first, second, and third substituent(s) and may enhance or improve the stability of a material and improve hole transport properties. Accordingly, the lifetime and emission efficiency of the light emitting element including the amine compound of one or more embodiments may be enhanced or improved. In some embodiments, the light emitting element of one or more embodiments may include the amine compound according to one or more embodiments in a hole transport layer to show enhanced or improved emission efficiency and lifetime characteristics.

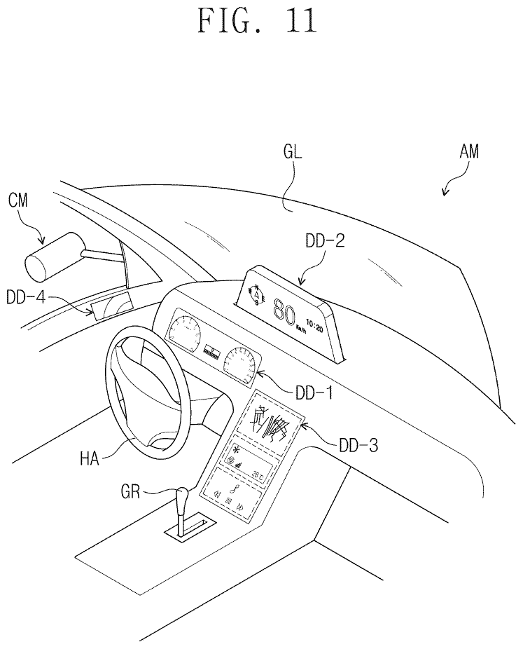

FIG. 11 is a diagram showing a vehicle (e.g., an automobile) AM in which first to fourth display devices DD-1, DD-2, DD-3 and DD-4 are provided. At least one among the first to fourth display devices DD-1, DD-2, DD-3 and DD-4 may include the same configurations of the display devices DD, DD-TD, DD-a, DD-b and DD-c of embodiments, explained referring to FIGS. 1, 2, and 7 to 10.

In FIG. 11, a vehicle is shown as an automobile AM, but this is an illustration, and the first to fourth display devices DD-1, DD-2, DD-3 and DD-4 may be provided on other transportation vehicles such as bicycles, motorcycles, trains, ships and airplanes. In some embodiments, at least one among the first to fourth display devices DD-1, DD-2, DD-3 and DD-4 including the same configurations of the display devices DD, DD-TD, DD-a, DD-b and DD-c may be introduced in personal computers, laptop computers, personal digital terminals, game consoles, portable electronic devices, televisions, monitors, external billboards, and/or the like. In some embodiments, these are suggested as examples, and the display device may be introduced in other electronic devices as long as not deviated from the present disclosure.

At least one among the first to fourth display devices DD-1, DD-2, DD-3 and DD-4 may include the light emitting element ED of one or more embodiments, explained referring to FIG. 3 to FIG. 6. The light emitting element ED of one or more embodiments may include the amine compound of one or more embodiments. At least one among the first to fourth display devices DD-1, DD-2, DD-3 and DD-4 may include the light emitting element ED including the amine compound of one or more embodiments and may show enhanced or improved display lifetime.

Referring to FIG. 11, an automobile AM may include a steering wheel HA for the operation of the automobile AM and a gear GR. In some embodiments, the automobile AM may include a front window GL provided to face a driver.

A first display device DD-1 may be provided in a first region overlapping with the steering wheel HA. For example, the first display device DD-1 may be a digital cluster displaying the first information of the automobile AM. The first information may include a first graduation showing the running speed of the automobile AM, a second graduation showing the number of revolution of an engine (i.e., revolutions per minute (RPM)), and images showing a fuel state. First graduation and second graduation may be represented by digital images.

A second display device DD-2 may be provided in a second region facing a driver's seat and overlapping with the front window GL. The driver's seat may be a seat where the steering wheel HA is provided. For example, the second display device DD-2 may be a head up display (HUD) showing the second information of the automobile AM. The second display device DD-2 may be optically clear. The second information may include digital numbers showing the running speed of the automobile AM and may further include information including the current time. Different from the drawing, the second information of the second display device DD-2 may be projected and displayed on the front window GL.

A third display device DD-3 may be provided in a third region adjacent to the gear GR. For example, the third display device DD-3 may be a center information display (CID) for an automobile, provided between a driver's seat and a passenger seat and showing third information. The passenger seat may be a seat separated from the driver's seat with the gear GR therebetween. The third information may include information on road conditions (for example, navigation information), on playing music or radio, on playing a dynamic image (or image), on the temperature in the automobile AM, and/or the like.

A fourth display device DD-4 may be provided in a fourth region separated from the steering wheel HA and the gear GR and adjacent to the side of the automobile AM. For example, the fourth display device DD-4 may be a digital wing mirror displaying fourth information. The fourth display device DD-4 may display the external image of the automobile AM, taken by a camera module CM provided at the outside of the automobile AM. The fourth information may include the external image of the automobile AM.

The described first to fourth information is for illustration, and the first to fourth display devices DD-1, DD-2, DD-3 and DD-4 may further display information on the inside and outside of the automobile. The first to fourth information may include different information from each other. However, one or more embodiments of the present disclosure is not limited thereto, and a portion of the first to fourth information may include the same information.

Terms such as “substantially,” “about,” and “approximately” are used as relative terms and not as terms of degree, and are intended to account for the inherent deviations in measured or calculated values that would be recognized by those of ordinary skill in the art. They may be inclusive of the stated value and an acceptable range of deviation as determined by one of ordinary skill in the art, considering the limitations and error associated with measurement of that quantity. For example, “about” may refer to one or more standard deviations, or +30%, 20%, 10%, 5% of the stated value.

Numerical ranges disclosed herein include and are intended to disclose all subsumed sub-ranges of the same numerical precision. For example, a range of “1.0 to 10.0” includes all subranges having a minimum value equal to or greater than 1.0 and a maximum value equal to or less than 10.0, such as, for example, 2.4 to 7.6. Applicant therefore reserves the right to amend this specification, including the claims, to expressly recite any sub-range subsumed within the ranges expressly recited herein.

The light emitting element, the display device and/or any other relevant devices or components according to embodiments of the present disclosure described herein may be implemented utilizing any suitable hardware, firmware (e.g., an application-specific integrated circuit), software, or a combination of software, firmware, and hardware. For example, the various components of the light emitting element and/or the display device may be formed on one integrated circuit (IC) chip or on separate IC chips. Further, the various components of the elements and/or devices may be implemented on a flexible printed circuit film, a tape carrier package (TCP), a printed circuit board (PCB), or formed on one substrate. Further, the various components of the elements and/or devices may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

In the present disclosure, when particles are spherical, “diameter” indicates an average particle diameter, and when the particles are non-spherical, the “diameter” indicates a major axis length. The diameter (or size) of the particles may be measured by particle size analysis, dynamic light scattering, scanning electron microscopy, and/or transmission electron microscope photography. When the size of the particles is measured utilizing a particle size analyzer, the average particle diameter (or size) may be referred to as D50. The term “D50” as utilized herein refers to the average diameter (or size) of particles whose cumulative volume corresponds to 50 vol % in the particle size distribution (e.g., cumulative distribution), and refers to the value of the particle size corresponding to 50% from the smallest particle when the total number of particles is 100% in the distribution curve accumulated in the order of the smallest particle size to the largest particle size. Particle size analysis may be performed with a HORIBA LA-950 laser particle size analyzer.

Hereinafter, referring to example embodiments and comparative example embodiments, the amine compound according to one or more embodiments and the light emitting element according to one or more embodiments of the present disclosure will be explained in particular. In some embodiments, the example embodiments are illustrations to assist the understanding of the present disclosure, but the scope of the present disclosure is not limited thereto.

### EXAMPLES

### 1. Synthesis of Amine Compounds

First, the synthetic methods of the amine compounds according to embodiments will be explained in particular illustrating the synthetic methods of Compound F18, Compound F8, Compound E11, Compound K8, Compound A18, Compound E7, Compound F14, and Compound F5. In some embodiments, the synthetic methods of the amine compounds explained hereinafter are embodiments, and the synthetic method of the amine compound according to one or more embodiments of the present disclosure is not limited to the embodiments.

### (1) Synthesis of Compound F18

Compound F18 according to one or more embodiments may be synthesized by, for example, Reaction 1-1 and Reaction 1-2.

### 1) Synthesis of Compound X3

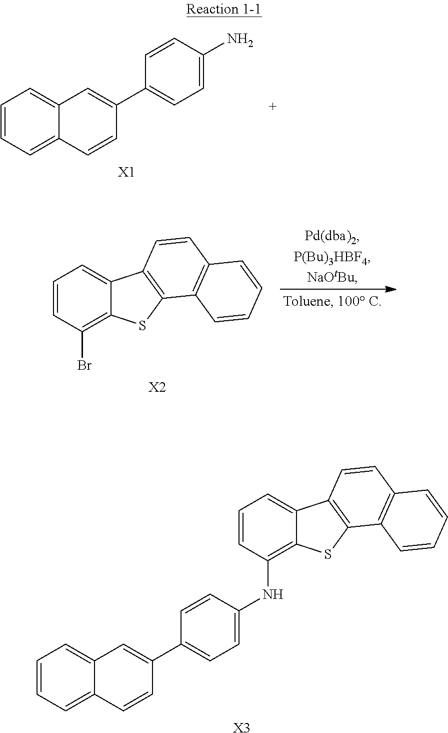

To Compound X1 (10 mmol), Compound X2 (10 mmol), NaO^{t}Bu (10 mmol), and P(Bu)₃HBF₄ (1 mmol), toluene (200 mL) was added and bubbles were removed. Under an argon atmosphere, bis(dibenzylideneacetone)palladium (0.5 mmol) was added thereto, followed by heating and stirring at about 100° C. for about 6 hours. The reaction solution was cooled to room temperature and extracted with toluene, and the resultant was washed with H₂O and brine, and dried over Na₂SO₄. The solution thus obtained was concentrated and purified by column chromatography to obtain Compound X3 (8.1 mmol, 81%, MS 451.14).

### 2) Synthesis of Compound F18

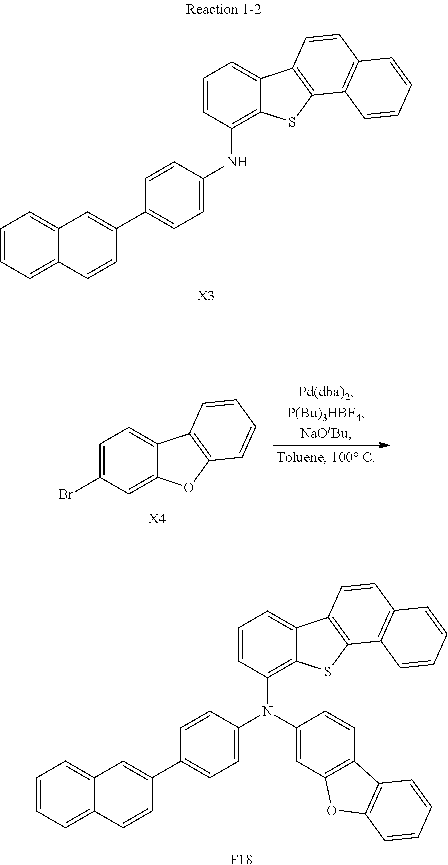

To Compound X3 (10 mmol), Compound X4 (10 mmol), NaO^{t}Bu (10 mmol), and P(Bu)₃HBF₄ (1 mmol), toluene (200 mL) was added and bubbles were removed. Under an argon atmosphere, bis(dibenzylideneacetone)palladium (0.5 mmol) was added thereto, followed by heating and stirring at about 100° C. for about 6 hours. The reaction solution was cooled to room temperature and extracted with toluene, and the resultant was washed with H₂O and brine, and dried over Na₂SO₄. The solution thus obtained was concentrated and purified by column chromatography to obtain Compound F18 (8.8 mmol, 88%, MS 617.18).

### (2) Synthesis of Compound F8

Compound F8 according to one or more embodiments may be synthesized by, for example, Reaction 2-1 and Reaction 2-2.

### 1) Synthesis of Compound X6

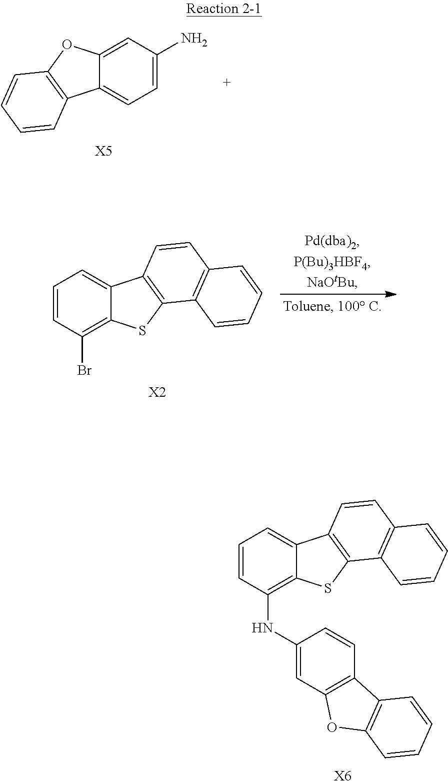 [SMILES: Nc1ccc2c(c1)oc1ccccc12 ; "X5" ; Brc1cccc2c1sc1c3ccccc3ccc21 ; "X2" ; c1ccc2c(c1)ccc1c3cccc(Nc4ccc5c(c4)oc4ccccc45)c3sc21 ; "X6"]

To Compound X5 (10 mmol), Compound X2 (10 mmol), NaO^{t}Bu (10 mmol), and P(Bu)₃HBF₄ (1 mmol), toluene (200 mL) was added and bubbles were removed. Under an argon atmosphere, bis(dibenzylideneacetone)palladium (0.5 mmol) was added thereto, followed by heating and stirring at about 100° C. for about 6 hours. The reaction solution was cooled to room temperature and extracted with toluene, and the resultant was washed with H₂O and brine, and dried over Na₂SO₄. The solution thus obtained was concentrated and purified by column chromatography to obtain Compound X6 (8.2 mmol, 82%, MS 415.10).

### 2) Synthesis of Compound F8

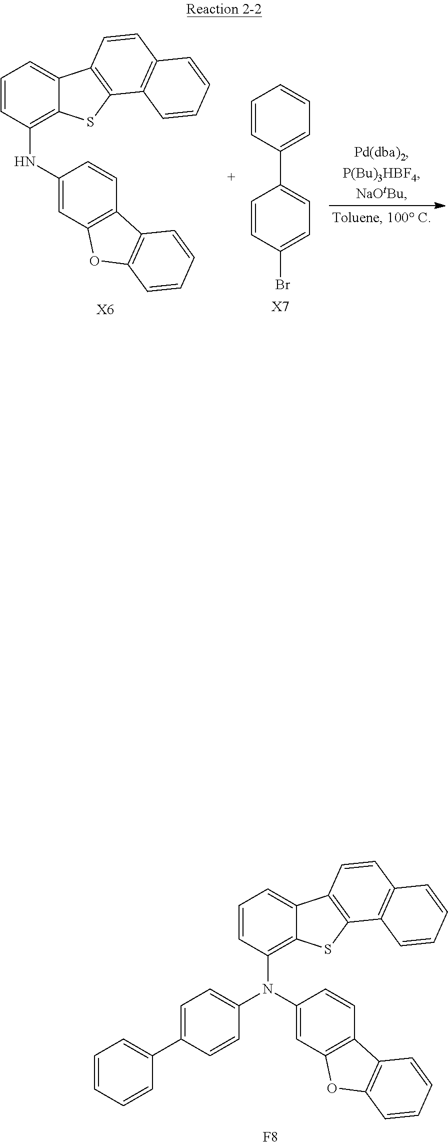

To Compound X6 (10 mmol), Compound X7 (10 mmol), NaO^{t}Bu (10 mmol), and P(Bu)₃HBF₄ (1 mmol), toluene (200 mL) was added and bubbles were removed. Under an argon atmosphere, bis(dibenzylideneacetone)palladium (0.5 mmol) was added thereto, followed by heating and stirring at about 100° C. for about 6 hours. The reaction solution was cooled to room temperature and extracted with toluene, and the resultant was washed with H₂O and brine, and dried over Na₂SO₄. The solution thus obtained was concentrated and purified by column chromatography to obtain Compound F8 (8.3 mmol, 83%, MS 567.17).

### (3) Synthesis of Compound E11

Compound E11 according to one or more embodiments may be synthesized by, for example, Reaction 3-1 and Reaction 3-2.

### 1) Synthesis of Compound X9

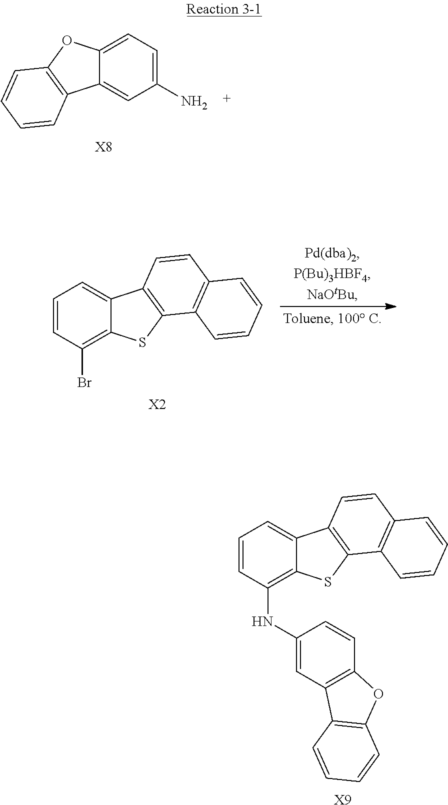

To Compound X8 (10 mmol), Compound X2 (10 mmol), NaO^{t}Bu (10 mmol), and P(Bu)₃HBF₄ (1 mmol), toluene (200 mL) was added and bubbles were removed. Under an argon atmosphere, bis(dibenzylideneacetone)palladium (0.5 mmol) was added thereto, followed by heating and stirring at about 100ºC for about 6 hours. The reaction solution was cooled to room temperature and extracted with toluene, and the resultant was washed with H₂O and brine, and dried over Na₂SO₄. The solution thus obtained was concentrated and purified by column chromatography to obtain Compound X9 (7.9 mmol, 79%, MS 415.10).

### 2) Synthesis of Compound E11

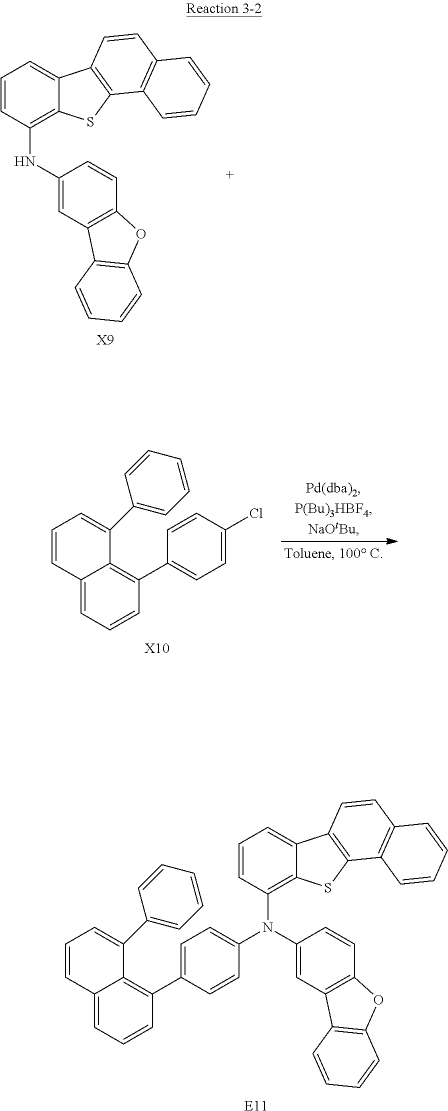

To Compound X9 (10 mmol), Compound X10 (10 mmol), NaO^{t}Bu (10 mmol), and P(Bu)₃HBF₄ (1 mmol), toluene (200 mL) was added and bubbles were removed. Under an argon atmosphere, bis(dibenzylideneacetone)palladium (0.5 mmol) was added thereto, followed by heating and stirring at about 100° C. for about 6 hours. The reaction solution was cooled to room temperature and extracted with toluene, and the resultant was washed with H₂O and brine, and dried over Na₂SO₄. The solution thus obtained was concentrated and purified by column chromatography to obtain Compound E11 (6.9 mmol, 69%, MS 693.21).

### (4) Synthesis of Compound K8

Compound K8 according to one or more embodiments may be synthesized by, for example, Reaction 4.

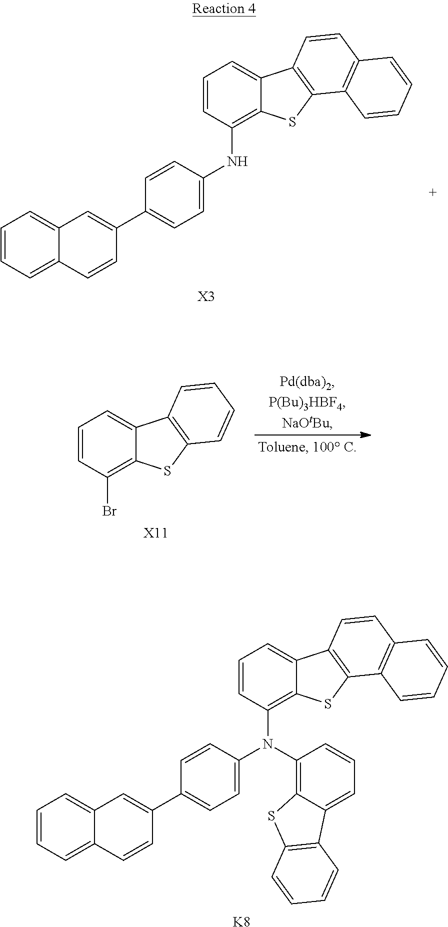 [SMILES: c1ccc2cc(-c3ccc(Nc4cccc5c4sc4c6ccccc6ccc54)cc3)ccc2c1 ; "X3" ; Brc1cccc2c1sc1ccccc12 ; "X11" ; c1ccc2cc(-c3ccc(N(c4cccc5c4sc4ccccc45)c4cccc5c4sc4c6ccccc6ccc54)cc3)ccc2c1 ; "K8"]

To Compound X3 (10 mmol), Compound X11 (10 mmol), NaO^{t}Bu (10 mmol), and P(Bu)₃HBF₄ (1 mmol), toluene (200 mL) was added and bubbles were removed. Under an argon atmosphere, bis(dibenzylideneacetone)palladium (0.5 mmol) was added thereto, followed by heating and stirring at about 100° C. for about 6 hours. The reaction solution was cooled to room temperature and extracted with toluene, and the resultant was washed with H₂O and brine, and dried over Na₂SO₄. The solution thus obtained was concentrated and purified by column chromatography to obtain Compound K8 (8.1 mmol, 81%, MS 583.14).

### (5) Synthesis of Compound A18

Compound A18 according to one or more embodiments may be synthesized by, for example, Reaction 5.

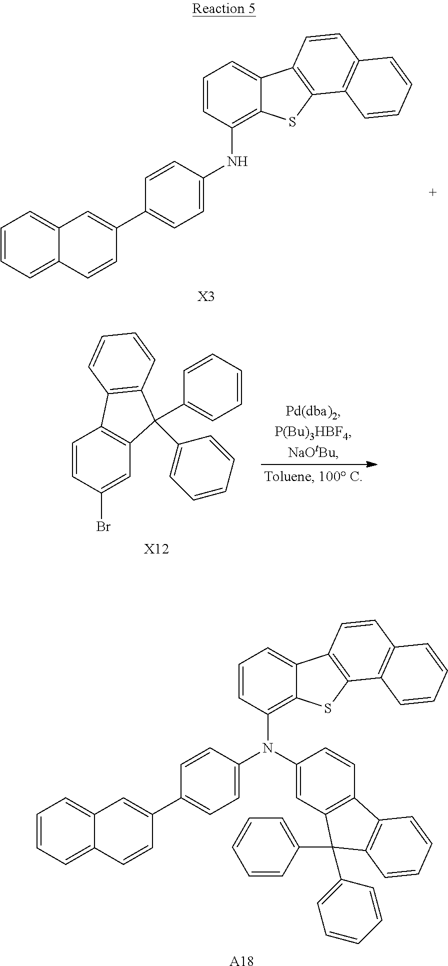 [SMILES: c1ccc2cc(-c3ccc(Nc4cccc5c4sc4c6ccccc6ccc54)cc3)ccc2c1 ; "X3" ; Brc1ccc2c(c1)C(c1ccccc1)(c1ccccc1)c1ccccc1-2 ; "X12" ; c1ccc(C2(c3ccccc3)c3ccccc3-c3ccc(N(c4ccc(-c5ccc6ccccc6c5)cc4)c4cccc5c4sc4c6ccccc6ccc54)cc32)cc1 ; "A18"]

To Compound X3 (10 mmol), Compound X12 (10 mmol), NaO^{t}Bu (10 mmol), and P(Bu)₃HBF₄ (1 mmol), toluene (200 mL) was added and bubbles were removed. Under an argon atmosphere, bis(dibenzylideneacetone)palladium (0.5 mmol) was added thereto, followed by heating and stirring at about 100ºC for about 6 hours. The reaction solution was cooled to room temperature and extracted with toluene, and the resultant was washed with H₂O and brine, and dried over Na₂SO₄. The solution thus obtained was concentrated and purified by column chromatography to obtain Compound A18 (8.9 mmol, 89%, MS 717.25).

### (6) Synthesis of Compound E7

Compound E7 according to one or more embodiments may be synthesized

by, for example, Reaction 6.

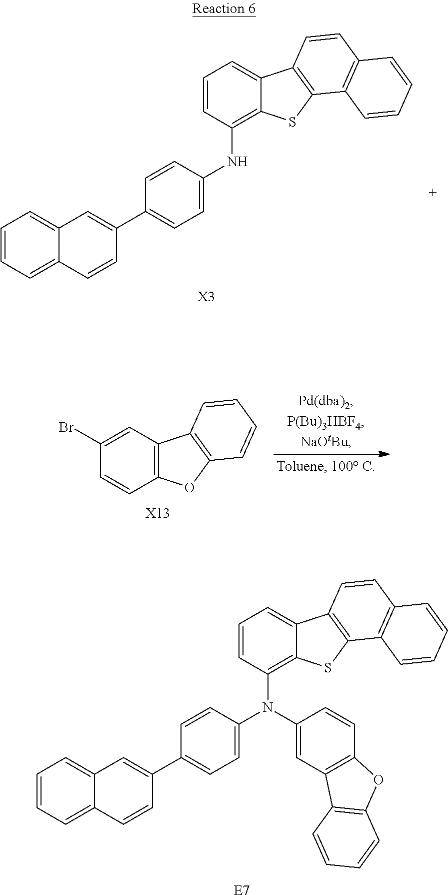

To Compound X3 (10 mmol), Compound X13 (10 mmol), NaO^{t}Bu (10 mmol), and P(Bu)₃HBF₄ (1 mmol), toluene (200 mL) was added and bubbles were removed. Under an argon atmosphere, bis(dibenzylideneacetone)palladium (0.5 mmol) was added thereto, followed by heating and stirring at about 100° C. for about 6 hours. The reaction solution was cooled to room temperature and extracted with toluene, and the resultant was washed with H₂O and brine, and dried over Na₂SO₄. The solution thus obtained was concentrated and purified by column chromatography to obtain Compound E7 (8.5 mmol, 85%, MS 567.17).

### (7) Synthesis of Compound F14

Compound F14 according to one or more embodiments may be synthesized by, for example, Reaction 7.

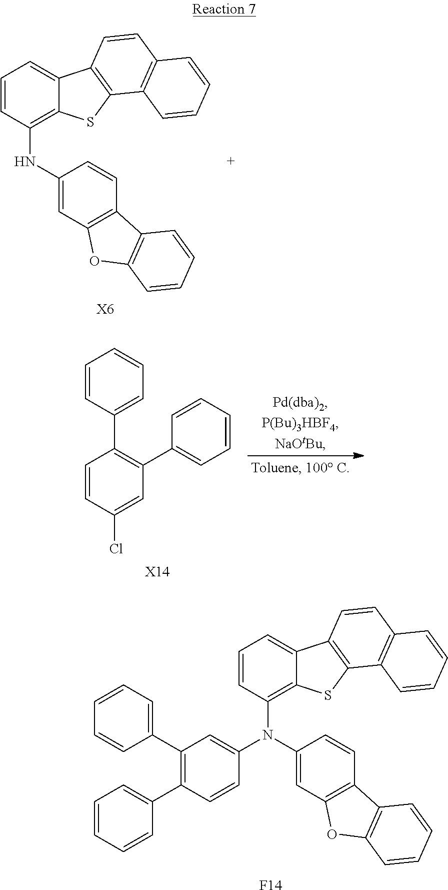

To Compound X6 (10 mmol), Compound X14 (10 mmol), NaO^{t}Bu (10 mmol), and P(Bu)₃HBF₄ (1 mmol), toluene (200 mL) was added and bubbles were removed. Under an argon atmosphere, bis(dibenzylideneacetone)palladium (0.5 mmol) was added thereto, followed by heating and stirring at about 100° C. for about 6 hours. The reaction solution was cooled to room temperature and extracted with toluene, and the resultant was washed with H₂O and brine, and dried over Na₂SO₄. The solution thus obtained was concentrated and purified by column chromatography to obtain Compound F14 (6.1 mmol, 61%, MS 643.20).

### (8) Synthesis of Compound F5

Compound F5 according to one or more embodiments may be synthesized by, for example, Reaction 8.

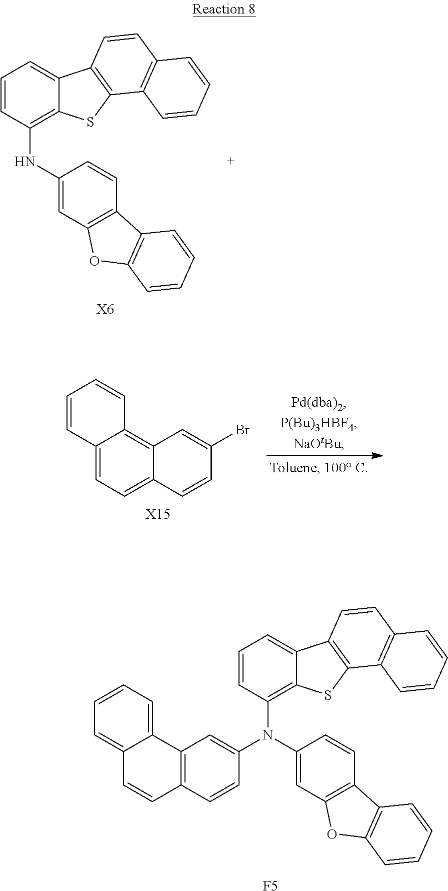

To Compound X6 (10 mmol), Compound X15 (10 mmol), NaO^{t}Bu (10 mmol), and P(Bu)₃HBF₄ (1 mmol), toluene (200 mL) was added and bubbles were removed. Under an argon atmosphere, bis(dibenzylideneacetone)palladium (0.5 mmol) was added thereto, followed by heating and stirring at about 100° C. for about 6 hours. The reaction solution was cooled to room temperature and extracted with toluene, and the resultant was washed with H₂O and brine, and dried over Na₂SO₄. The solution thus obtained was concentrated and purified by column chromatography to obtain Compound F5 (8.8 mmol, 88%, MS 591.17).

### 2. Manufacture and Evaluation of Light Emitting Elements

A light emitting element of one or more embodiments including the amine compound of one or more embodiments in a hole transport layer was manufactured by a method. Light emitting elements of Example 1 to Example 8 were manufactured utilizing the amine compounds of Compound F18, Compound F8, Compound E11, Compound K8, Compound A18, Compound E7, Compound F14 and Compound F5, which are the above-explained Example Compounds, as hole transport layer materials. Comparative Example 1 to Comparative Example 10 correspond to light emitting elements manufactured utilizing Comparative Compounds R1 to R10 as hole transport layer materials.

Example Compounds

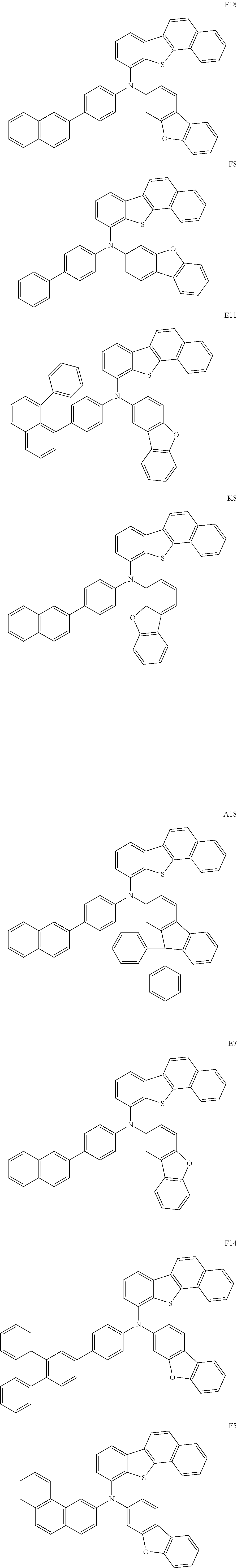

Comparative Compounds

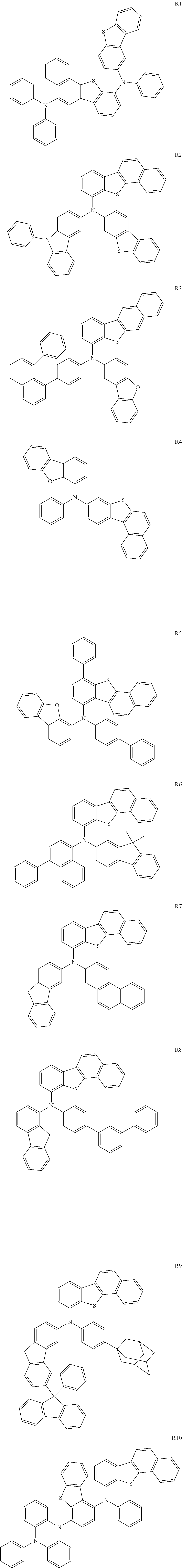

### Manufacture of Light Emitting Elements

An ITO glass substrate with about 15 ohm per square centimeter (Ω/cm²) (about 150 nm) of Corning Co. was cut into a size of 50 millimeter (mm)×50 mm×0.7 mm, washed with isopropyl alcohol and ultrapure water, cleansed utilizing ultrasonic waves for about 5 minutes, exposed to UV for about 30 minutes and treated with ozone. Then, 4,4′,4″-tris{N-(2-naphthyl)-N-phenylamino}-triphenylamine (2-TNATA) was vacuum deposited to a thickness of about 60 nm to form a hole injection layer. After that, the Example Compound or Comparative Compound was vacuum deposited to a thickness of about 30 nm to form a hole transport layer.

On the hole transport layer, a blue fluorescence host of 9,10-di(naphthalen-2-yl)anthracene (ADN) and a fluorescence dopant of 2,5,8,11-tetra-t-butylperylene (TBP) were co-deposited in a ratio (e.g., amount) of about 97:3 to form an emission layer with a thickness of about 25 nm.

On the emission layer, an electron transport layer was formed to a thickness of about 250 nm utilizing tris(8-hydroxyquinolino)aluminum (Alq₃), and then, an electron injection layer was formed to a thickness of about 1 nm by depositing LiF. On the electron injection layer, a second electrode was formed to a thickness of about 100 nm utilizing aluminum (Al).

In some embodiments, the compounds of the functional layers utilized for the manufacture of the light emitting elements are as follows.

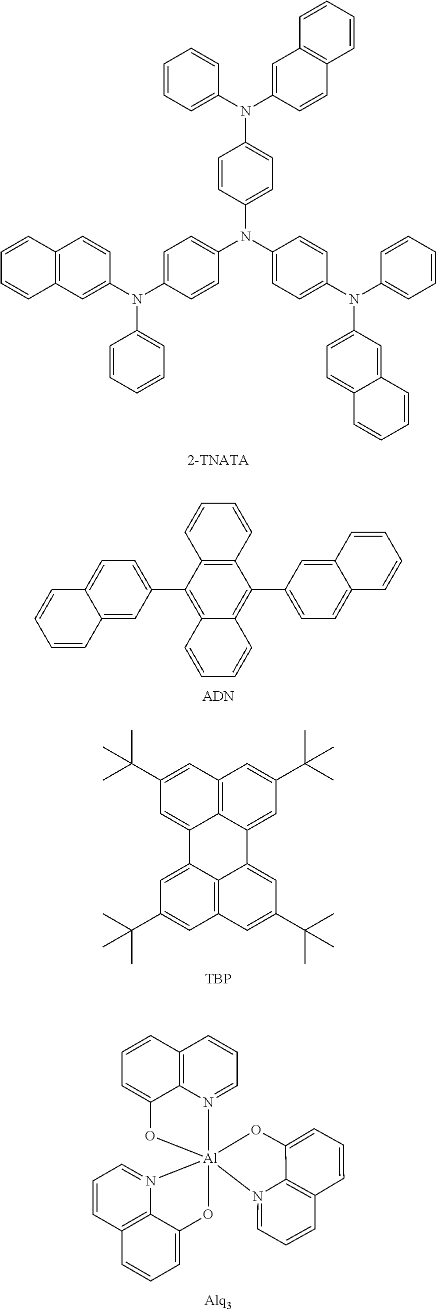

### Evaluation of Light Emitting Elements

Table 1 shows evaluation results on the light emitting elements of Examples 1 to 8, and Comparative Examples 1 to 10. In Table 1, the evaluation results on the lifetime of the light emitting elements manufactured are shown.

In the evaluation results of the properties of the Examples and Comparative Examples, shown in Table 1, the element lifetime (%) shows relative values, i.e., relative lifetime with respect to the element lifetime of Comparative Example 8 of 100%.

TABLE 1

| | Hole Transport | | | Hole Transport | |
|---|---|---|---|---|---|
| | Layer | | | Layer | |
| | (Example | Lifetime | Comparative | (Comparative | Lifetime |
| Example ID | Compound ID) | (%) | Example ID | Compound ID) | (%) |
| Example 1 | F18 | 113 | 1 | R1 | 81 |
| Example 2 | F8 | 110 | 2 | R2 | 89 |
| Example 3 | E11 | 112 | 3 | R3 | 97 |
| Example 4 | K8 | 109 | 4 | R4 | 96 |
| Example 5 | A18 | 111 | 5 | R5 | 99 |
| Example 6 | E7 | 115 | 6 | R6 | 91 |
| Example 7 | F14 | 113 | 7 | R7 | 92 |
| Example 8 | F5 | 114 | 8 | R8 | 100 |
| | | | 9 | R9 | 86 |
| | | | 10 | R10 | 88 |

Referring to the results of Table 1, it could be found that the light emitting elements of the Examples utilizing the amine compounds according to embodiments of the present disclosure as the hole transport layer materials, showed relatively long element lifetime when compared to the Comparative Examples.

The amine compound of one or more embodiments according to the present disclosure includes first to third substituents, directly or indirectly connected with the nitrogen atom of an amine. For example, the amine compound of one or more embodiments includes a first substituent of a benzonaphthothiophene group, and a second substituent of a dibenzoheterole group or a fluorenyl group, and may contribute to the long-life (e.g., long lifetime) characteristics of the light emitting element. The amine compound of one or more embodiments may induce the improvement of stacking and orientation due to the intermolecular interaction of the first substituent and the second substituent, which have similar skeletons. Accordingly, the light emitting element including the amine compound of one or more embodiments may show improved lifetime characteristics.

In Example 1 to Example 8 compared to Comparative Example 1, the light emitting elements of the Examples showed increased element lifetime and improved results of element properties. Comparative Compound R1 included in Comparative Example 1 is a diarylamine compound, and showed electronic effects and steric effects around two nitrogen atoms different from the Example Compounds, and it is thought that the improving effects of the lifetime of the element was suppressed or reduced compared to the light emitting elements of the Examples.

Comparative Compound R2 included in Comparative Example 2 included a carbazole group around the nitrogen atom of an amine and showed degraded results of the lifetime of the light emitting element. When a carbazole group connected with the nitrogen atom of an amine is included as in Comparative Compound R2, it is thought that the carbazole group having relatively strong electron donor properties dominates intermolecular interaction, to suppress or reduce the improving effects of lifetime through the composition of a benzonaphthothiophene group and a dibenzoheterole group.

Comparative Example 3 and Comparative Example 4 include Comparative Compound R3 and Comparative Compound R4, respectively, and showed degraded results of the lifetime of the light emitting elements in contrast to Example 1 to Example 3. From such results, it could be found that the improvement of the lifetime of the light emitting element could be accomplished when the benzonaphthothiophene group connected with the nitrogen atom of an amine is benzo[b]naphtha[2,1-d]thiophene group, and the improving effects of the lifetime could not be shown in another fused ring structure. The benzonaphthothiophene group included in the amine compound of one or more embodiments has a structure in which a fused ring is formed at the α and β positions of an electronically different naphthyl groups, and a sulfur atom is substituted at the electron-rich a position of the naphthyl group rather than the β position, and it is thought that the electronic effects of the benzonaphthothiophene group having a specific structure influenced the properties of the light emitting element.

Comparative Example 3 to Comparative Example 5 shows deteriorated lifetime characteristics when compared to Example 1 to Example 4. The Example Compounds are thought to show the improving effects of the lifetime through the connection of the benzonaphthothiophene group with the nitrogen atom of an amine at carbon position 10 when compared to Comparative Compounds R3 to R5, included in Comparative Examples 3 to 5. In some embodiments, the Example Compounds are thought to show the improving effects of orientation, and/or the like through the positioning of the sulfur atom of the benzonaphthothiophene group at the ortho relation with respect to the nitrogen atom of the amine.

Comparative Example 6 showed degraded lifetime characteristics when compared to Example 1, Example 2 and Example 5. In Comparative Compound R6 included in Comparative Example 6, a substituent having low stability like a methyl group is substituted for the fluorenyl group at the nitrogen atom of an amine, and it is thought that suitable molecular orientation was not maintained, and the improvement of the lifetime of the light emitting element was not achieved.

Comparative Example 7 shows degraded lifetime characteristics when compared to Example 1 to Example 8. In Comparative Compound R7 included in Comparative Example 7, a 2-phenanthorene group is included around the nitrogen atom of an amine to dominate the intermolecular interaction at the 2-phenanthorene group. Accordingly, it is thought that the characteristic improving effects of the lifetime through the composition of the benzonaphthothiophene group and the dibenzoheterole group were suppressed or reduced.

For example, the characteristic improving effects of the lifetime through the composition of the benzonaphthothiophene group and the dibenzoheterole group in the amine compound of one or more embodiments may be shown in case of introducing a phenanthrenyl group around the nitrogen atom of an amine by the position selective effects. When comparing Example Compound 8 included in Example 8 and Comparative Compound R7 included in Comparative Example 7, it is thought that the improving effects of stacking and orientation by the intermolecular interaction of the benzonaphthothiophene group and the dibenzoheterole group are restrained or improved according to the position of a phenanthrenyl group connected with the nitrogen atom of an amine.

Comparative Example 8 shows degraded lifetime characteristics when compared to Example 1 to Example 8. It is thought that Comparative Compound R8 included in Comparative Example 8 includes a linear terphenyl group, and the improvement of stacking and orientation were suppressed or reduced by the intermolecular interaction of the benzonaphthothiophene group and the dibenzoheterole group. In Comparative Compound R8, it is thought that the linear terphenyl group having a relatively high degree of freedom of rotation could take lots of thermodynamically local structures to suppress or reduce the characteristic intermolecular interaction of the benzonaphthothiophene group and the dibenzoheterole group.

For example, in the amine compound of one or more embodiments, represented by Formula 1, when X is O or S and is at the ortho position or para position with respect to the nitrogen atom of an amine, insufficient tendency of stability in a molecule may be added, and the improvement of the lifetime of the light emitting element may be markedly suppressed or reduced.

Comparative Example 9 and Comparative Example 10 show degraded lifetime characteristics when compared to Example 1 to Example 8. As in Comparative Compound R9 included in Comparative Example 9, it is thought that when a bulky substituent like a fluorenyl group is introduced into a dibenzoheterole group, the intermolecular interaction between the benzonaphthothiophene group and the dibenzoheterole group may be suppressed or reduced to show degraded lifetime characteristics.

When a substituent having high electron donating properties like pyridine is introduced as in Comparative Compound R10 included in Comparative Example 10, it is thought that intermolecular interaction among pyridine groups may be strengthened, and the improving effects of the lifetime of the light emitting element may be suppressed or reduced.

The light emitting element of one or more embodiments includes the amine compound of one or more embodiments and may show long-life (e.g., long lifetime) characteristics.

In a case where the amine compound of one or more embodiments is applied to a light emitting element, long-life (e.g., long lifetime) characteristics may be shown.

Although the embodiments of the present disclosure have been described, it will be understood by those skilled in the art that the present disclosure should not be limited to these embodiments, but one or more suitable changes and modifications can be made by one of ordinary skill in the art within the spirit and scope of the present disclosure as set forth in the following claims and equivalents thereof.

Accordingly, the technical scope of the present disclosure is not to be limited to the contents set forth in the detailed description of the specification, but is intended to be defined by the appended claims, and equivalents thereof.

What is claimed is:

1. A light emitting element, comprising:
a first electrode;
a second electrode on the first electrode; and
at least one functional layer between the first electrode and the second electrode,
wherein the at least one functional layer comprises an amine compound represented by Formula 1: [symbol]
, and Formula 1
in Formula 1,
R₁ and R₂ are each independently a hydrogen atom, a deuterium atom, a substituted or unsubstituted alkyl group of 1 to 10 carbon atoms, a substituted or unsubstituted aryl group of 6 to 30 ring-forming carbon atoms, or a substituted or unsubstituted heteroaryl group of 5 to 30 ring-forming carbon atoms,
L is a direct linkage, a substituted or unsubstituted arylene group of 6 to 30 ring-forming carbon atoms, or a substituted or unsubstituted heteroarylene group of 5 to 30 ring-forming carbon atoms,
“a” is an integer of 0 to 3, “b” is an integer of 0 to 6,
Ar₁ is represented by Formula 2,
Ar₂ is a substituted or unsubstituted aryl group of 6 to 30 ring-forming carbon atoms, or a substituted or unsubstituted heteroaryl group of 5 to 30 ring-forming carbon atoms, and
Ar₂ is not a substituted or unsubstituted carbazole group: [symbol]
, and Formula 2
in Formula 2,
X is O, S, or CR₅R₆,
R₃ and R₄ are each independently a hydrogen atom, a deuterium atom, a substituted or unsubstituted alkyl group of 1 to 10 carbon atoms, a substituted or unsubstituted aryl group of 6 to 30 ring-forming carbon atoms, or a substituted or unsubstituted heteroaryl group of 5 to 30 ring-forming carbon atoms,
R₅ and R₆ are each independently a substituted or unsubstituted aryl group of 6 to 30 ring-forming carbon atoms, or a substituted or unsubstituted heteroaryl group of 5 to 30 ring-forming carbon atoms,
“c” is an integer of 0 to 3, “d” is an integer of 0 to 4,
when X is O or S and is at an ortho position or a para position with respect to “N” in Formula 1, [symbol] is not an unsubstituted phenyl group, an unsubstituted biphenyl group, an unsubstituted terphenyl group, or an unsubstituted 2-phenanthrenyl group, and
each of R₃ and R₄ is not a substituted or unsubstituted 9-fluorenyl group, or a substituted or unsubstituted phenazine group.

2. The light emitting element of claim 1, wherein the amine compound represented by Formula 1 is represented by any one selected from among Formula 3-1 to Formula 3-3: [symbol]
Formula 3-1 [symbol]
Formula 3-2 [symbol]
, and Formula 3-3
in Formula 3-2 and Formula 3-3,
R_{I1} and R_{I2} are each independently a hydrogen atom, a deuterium atom, a substituted or unsubstituted alkyl group of 1 to 10 carbon atoms, a substituted or unsubstituted aryl group of 6 to 30 ring-forming carbon atoms, or a substituted or unsubstituted heteroaryl group of 5 to 30 ring-forming carbon atoms,
I1 is an integer of 0 to 4,
I2 is an integer of 0 to 6, and
“n” is an integer of 1 to 3, and
in Formula 3-1 to Formula 3-3,
R₁, R₂, Ar₁, Ar₂, “a” and “b” are each independently as defined in Formula 1.

3. The light emitting element of claim 1, wherein the amine compound represented by Formula 1 is represented by any one selected from among Formula 4-1 to Formula 4-3: [symbol]
Formula 4-1 [symbol]
Formula 4-2 [symbol]
, and Formula 4-3
in Formula 4-1 to Formula 4-3,
R₁₁ to R₁₄, R₂₁ to R₂₄, and R₃₁ to R₃₆ are each independently a hydrogen atom or a deuterium atom,
a1 to a3, and c1 to c3 are each independently an integer of 0 to 3,
b1 to b3 are each independently an integer of 0 to 6,
d1 to d3 are each independently an integer of 0 to 4,
“i” and “j” are each independently an integer of 0 to 5, and
Ar₂ and L are each independently as defined in Formula 1.

4. The light emitting element of claim 1, wherein the amine compound represented by Formula 1 is represented by any one selected from among Formula 5-1 or Formula 5-2: [symbol]
Formula 5-1 [symbol]
, and Formula 5-2
in Formula 5-1,
Rₐ to Rₑ are each independently a hydrogen atom, a deuterium atom, or a substituted or unsubstituted aryl group of 6 to 20 ring-forming carbon atoms, and/or Rₐ and R_{b} form a substituted or unsubstituted aromatic hydrocarbon ring, R_{b} and R_{c} form a substituted or unsubstituted aromatic hydrocarbon ring, R_{c} and R_{d} form a substituted or unsubstituted aromatic hydrocarbon ring, and/or R_{d} and Rₑ form a substituted or unsubstituted aromatic hydrocarbon ring,
in Formula 5-2,
Y is O, S or CR_{y1}R_{y2},
R_{f} and R_{g} are each independently a hydrogen atom or a deuterium atom,
R_{y1} and R_{y2} are each independently a substituted or unsubstituted alkyl group of 1 to 10 carbon atoms, or a substituted or unsubstituted aryl group of 6 to 15 ring-forming carbon atoms, and/or R_{y1} and R_{y2} form a ring,
“f” is an integer of 0 to 4, “g” is an integer of 0 to 3, and
in Formula 5-1 and Formula 5-2,
R₁, R₂, L, Ar₁, “a” and “b” are each independently as defined in Formula 1.

5. The light emitting element of claim 1, wherein Ar₂ is a substituted or unsubstituted phenyl group, a substituted or unsubstituted biphenyl group, a substituted or unsubstituted terphenyl group, a substituted or unsubstituted quaterphenyl group, a substituted or unsubstituted naphthyl group, a substituted or unsubstituted phenanthrenyl group, a substituted or unsubstituted dibenzofuran group, a substituted or unsubstituted dibenzothiophene group, or a substituted or unsubstituted fluorenyl group.

6. The light emitting element of claim 1, wherein the amine compound represented by Formula 1 is a monoamine compound.

7. The light emitting element of claim 1, wherein Ar₁ is represented by any one selected from among Substituent Group 1: [symbol]
. Substituent Group 1

8. The light emitting element of claim 1, wherein [symbol] is represented by any one selected from among Substituent Group 2: [symbol] [symbol] [symbol] [symbol]
. Substituent Group 2

9. The light emitting element of claim 1, wherein R₁ and R₂ are hydrogen atoms.

10. The light emitting element of claim 1, wherein the at least one functional layer comprises:
a hole transport region on the first electrode;
an emission layer on the hole transport region; and
an electron transport region on the emission layer, and
the hole transport region comprises the amine compound represented by Formula 1.

11. The light emitting element of claim 10, wherein the hole transport region comprises:
a hole injection layer on the first electrode; and
a hole transport layer on the hole injection layer, and
the hole transport layer comprises the amine compound represented by Formula 1.

12. The light emitting element of claim 10, wherein the emission layer comprises a compound represented by Formula E-1: [symbol]
, and Formula E-1
in Formula E-1,
R₃₁ to R₄₀ are each independently a hydrogen atom, a deuterium atom, a halogen atom, a substituted or unsubstituted silyl group, a substituted or unsubstituted thio group, a substituted or unsubstituted oxy group, a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring, and
“c” and “d” are each independently an integer of 0 to 5.

13. An amine compound represented by Formula 1: [symbol]
, and Formula 1
in Formula 1,
R₁ and R₂ are each independently a hydrogen atom, a deuterium atom, a substituted or unsubstituted alkyl group of 1 to 10 carbon atoms, a substituted or unsubstituted aryl group of 6 to 30 ring-forming carbon atoms, or a substituted or unsubstituted heteroaryl group of 5 to 30 ring-forming carbon atoms,
L is a direct linkage, a substituted or unsubstituted arylene group of 6 to 30 ring-forming carbon atoms, or a substituted or unsubstituted heteroarylene group of 5 to 30 ring-forming carbon atoms,
“a” is an integer of 0 to 3, “b” is an integer of 0 to 6,
Ar₁ is represented by Formula 2,
Ar₂ is a substituted or unsubstituted aryl group of 6 to 30 ring-forming carbon atoms, or a substituted or unsubstituted heteroaryl group of 5 to 30 ring-forming carbon atoms, and
Ar₂ is not a substituted or unsubstituted carbazole group: [symbol]
, and Formula 2
in Formula 2,
X is O, S, or CR₅R₆,
R₃ and R₄ are each independently a hydrogen atom, a deuterium atom, a substituted or unsubstituted alkyl group of 1 to 10 carbon atoms, a substituted or unsubstituted aryl group of 6 to 30 ring-forming carbon atoms, or a substituted or unsubstituted heteroaryl group of 5 to 30 ring-forming carbon atoms,
R₅ and R₆ are each independently a substituted or unsubstituted aryl group of 6 to 30 ring-forming carbon atoms, or a substituted or unsubstituted heteroaryl group of 5 to 30 ring-forming carbon atoms,
“c” is an integer of 0 to 3, “d” is an integer of 0 to 4,
when X is O or S and is at an ortho position or a para position with respect to a “N” in Formula 1, [symbol] is not an unsubstituted phenyl group, an unsubstituted biphenyl group, an unsubstituted terphenyl group, or an unsubstituted 2-phenanthrenyl group, and
each of R₃ and R₄ is not a substituted or unsubstituted 9-fluorenyl group, or a substituted or unsubstituted phenazine group.

14. The amine compound of claim 13, wherein Formula 1 is represented by any one selected from among Formula 3-1 to Formula 3-3: [symbol]
Formula 3-1 [symbol]
Formula 3-2 [symbol]
,and Formula 3-3
in Formula 3-2 and Formula 3-3,
R_{I1} and R_{I2} are each independently a hydrogen atom, a deuterium atom, a substituted or unsubstituted alkyl group of 1 to 10 carbon atoms, a substituted or unsubstituted aryl group of 6 to 30 ring-forming carbon atoms, or a substituted or unsubstituted heteroaryl group of 5 to 30 ring-forming carbon atoms,
I1 is an integer of 0 to 4,
I2 is an integer of 0 to 6, and
“n” is an integer of 1 to 3, and
in Formula 3-1 to Formula 3-3,
R₁, R₂, Ar₁, Ar₂, “a” and “b” are each independently as defined in Formula 1.

15. The amine compound of claim 13, wherein Formula 1 is represented by any one selected from among Formula 4-1 to Formula 4-3: [symbol]
Formula 4-1 [symbol]
Formula 4-2 [symbol]
,and Formula 4-3
in Formula 4-1 to Formula 4-3,
R₁₁ to R₁₄, R₂₁ to R₂₄, and R₃₁ to R₃₆ are each independently a hydrogen atom or a deuterium atom,
a1 to a3, and c1 to c3 are each independently an integer of 0 to 3,
b1 to b3 are each independently an integer of 0 to 6,
d1 to d3 are each independently an integer of 0 to 4,
“i” and “j” are each independently an integer of 0 to 5, and
Ar₂ and L are each independently as defined in Formula 1.

16. The amine compound of claim 13, wherein Formula 1 is represented by any one selected from among Formula 5-1 or Formula 5-2: [symbol]
Formula 5-1 [symbol]
,and Formula 5-2
in Formula 5-1,
Rₐ to Rₑ are each independently a hydrogen atom, a deuterium atom, or a substituted or unsubstituted aryl group of 6 to 20 ring-forming carbon atoms, and/or Rₐ and R_{b} form a substituted or unsubstituted aromatic hydrocarbon ring, R_{b} and R_{c} form a substituted or unsubstituted aromatic hydrocarbon ring, R_{c} and Rₐ form a substituted or unsubstituted aromatic hydrocarbon ring, and/or Rₐ and Rₑ form a substituted or unsubstituted aromatic hydrocarbon ring,
in Formula 5-2,
Y is O, S or CR_{y1}R_{y2},
R_{f} and R_{g} are each independently a hydrogen atom or a deuterium atom,
R_{y1} and R_{y2} are each independently a substituted or unsubstituted alkyl group of 1 to 10 carbon atoms, or a substituted or unsubstituted aryl group of 6 to 15 ring-forming carbon atoms, and/or R_{y1} and R_{y2} form a ring,
“f” is an integer of 0 to 4, and “g” is an integer of 0 to 3, and
in Formula 5-1 and Formula 5-2,
R₁, R₂, L, Ar₁, “a” and “b” are each independently as defined in Formula 1.

17. The amine compound of claim 13, wherein Ar₂ is a substituted or unsubstituted phenyl group, a substituted or unsubstituted biphenyl group, a substituted or unsubstituted terphenyl group, a substituted or unsubstituted quaterphenyl group, a substituted or unsubstituted naphthyl group, a substituted or unsubstituted phenanthrenyl group, a substituted or unsubstituted dibenzofuran group, a substituted or unsubstituted dibenzothiophene group, or a substituted or unsubstituted fluorenyl group.

18. The amine compound of claim 13, wherein Ar₁ is represented by any one selected from among Substituent Group 1: [symbol]
. Substituent Group 1

19. The amine compound of claim 13, wherein [symbol] is represented by any one selected from among Substituent Group 2: [symbol] [symbol] [symbol] [symbol]
. Substituent Group 2

20. The amine compound of claim 13, wherein Formula 1 is represented by Formula 1-a, and
the amine compound represented by Formula 1-a satisfies any one selected from among combinations represented in Compound Combination Table 1: [symbol]
, and Formula 1-a
in Formula 1-a,
Fa is represented by Substituent fa1, Fb is selected from Substituent Group 1, and Fc is selected from Substituent Group 2: [symbol]
Substituent fa1 [symbol]
Substituent Group 1 [symbol] [symbol] [symbol] [symbol]
Substituent Group 2 [symbol] [symbol] [symbol] [symbol] [symbol] [symbol] [symbol] [symbol] [symbol] [symbol] [symbol] [symbol] [symbol] [symbol] [symbol] [symbol]
Compound Combination Table 1
